SEQUENCE LISTING (63 sequences)

SEQ ID NO: 1 - DNA, 20 bp, Artificial Sequence
  Oligomeric compound
tccgtcatcgctcctcaggg

SEQ ID NO: 2 - DNA, 20 bp, Artificial Sequence
  Oligomeric compound
gtgcgcgcgagcccgaaatc

SEQ ID NO: 3 - DNA, 20 bp, Artificial Sequence
  Oligomeric compound
atgcattctgcccccaagga

SEQ ID NO: 4 - DNA, 3160 bp, H. sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 5 - DNA, 26 bp, Artificial Sequence
  PCR primer
aatggctaagtgaagatgacaatcat

SEQ ID NO: 6 - DNA, 25 bp, Artificial Sequence
  PCR primer
tgcacatatcattacaccagttcgt

SEQ ID NO: 7 - DNA, 30 bp, Artificial Sequence
  PCR probe
ttgcagcaattcactgtaaagctggaaagg

SEQ ID NO: 8 - DNA, 1619 bp, H. sapiens
ccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcctgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttc

SEQ ID NO: 9 - DNA, 22 bp, Artificial Sequence
  PCR primer
caccacttccagggtttattcc

SEQ ID NO: 10 - DNA, 25 bp, Artificial Sequence
  PCR primer
tgatctcctttcctaagacattgct

SEQ ID NO: 11 - DNA, 22 bp, Artificial Sequence
  PCR probe
accagccttcctgtgggcccct

SEQ ID NO: 12 - DNA, 1842 bp, H. sapiens
cgatcagatcgatctaagatggcgactgtcgaaccggaaaccacccctactcctaatcccccgactacagaagaggagaaaacggaatctaatcaggaggttgctaacccagaacactatattaaacatcccctacagaacagatgggcactctggttttttaaaaatgataaaagcaaaacttggcaagcaaacctgcggctgatctccaagtttgatactgttgaagacttttgggctctgtacaaccatatccagttgtctagtaatttaatgcctggctgtgactactcactttttaaggatggtattgagcctatgtgggaagatgagaaaaacaaacggggaggacgatggctaattacattgaacaaacagcagagacgaagtgacctcgatcgcttttggctagagacacttctgtgccttattggagaatcttttgatgactacagtgatgatgtatgtggcgctgttgttaatgttagagctaaaggtgataagatagcaatatggactactgaatgtgaaaacagagaagctgttacacatatagggagggtatacaaggaaaggttaggacttcctccaaagatagtgattggttatcagtcccacgcagacacagctactaagagcggctccaccactaaaaataggtttgttgtttaagaagacaccttctgagtattctcataggagactgcgtcaagcaatcgagatttgggagctgaaccaaagcctcttcaaaaagcagagtggactgcatttaaatttgatttccatcttaatgttactcagatataagagaagtctcattcgcctttgtcttgtacttctgtgttcattttttttttttttttttggctagagtttccactatcccaatcaaagaattacagtacacatccccagaatccataaatgtgttcctggcccactctgtaatagttcagtagaattaccattaattacatacagattttacctatccacaatagtcagaaaacaacttggcatttctatactttacaggaaaaaaaattctgttgttccattttatgcagaagcatattttgctggtttgaaagattatgatgcatacagttttctagcaattttctttgtttctttttacagcattgtctttgctgtactcttgctgatggctgctagattttaatttatttgtttccctacttgataatattagtgattctgatttcagtttttcatttgttttgcttaaattttttttttttttttcctcatgtaacattggtgaaggatccaggaatatgacacaaaggtggaataaacattaattttgtgcattctttggtaattttttttgttttttgtaactacaaagctttgctacaaatttatgcatttcattcaaatcagtgatctatgtttgtgtgatttcctaaacataattgtggattataaaaaatgtaacatcataattacattcctaactagaattagtatgtctgtttttgtatctttatgctgtattttaacactttgtattacttaggttattttgctttggttaaaaatggctcaagtagaaaagcagtcccattcatattaagacagtgtacaaaactgtaaataaaatgtgtacagtgaattgtcttttagacaactagatttgtccttttatttctccatctttatagaaggaatttgtacttcttattgcaggcaagtctctatattatgtcctcttttgtggtgtcttccatgtgaacagcataagtttggagcactagtttgattattatgtttattacaatttttaataaattgaataggtagtatcatatatatgga

SEQ ID NO: 13 - DNA, 17 bp, Artificial Sequence
  PCR primer
tggcgactgtcgaaccg

SEQ ID NO: 14 - DNA, 26 bp, Artificial Sequence
  PCR primer
agattccgttttctcctcttctgtag

SEQ ID NO: 15 - DNA, 26 bp, Artificial Sequence
  PCR probe
aaaccacccctactcctaatcccccg

SEQ ID NO: 16 - DNA, 2763 bp, M. musculus
gagatcgatctaagatggcgactgtggaaccggaaaccacccctaccactaatcccccacctgcagaagaggaaaaaacagagtctaatcaagaggttgctaacccagagcactatattaaacaccctctacagaacaggtgggcactctggttttttaaaaatgataaaagcaaaacttggcaagcaaaccttcgattgatctctaagtttgatactgttgaagacttttgggctctatacaaccatatccagttgtctagtaatttaatgcctggctgtgactactcactttttaaggacgggattgagcctatgtgggaagatgagaaaaacaaacgaggaggacggtggctgatcacactgaacaagcagcagagacggagtgacctcgatcgcttctggctagagacactgctgtgccttattggagaatctttcgatgactacagtgatgatgtgtgtggagctgttgttaatgttagagctaaaggcgataagatagcaatatggactactgagagtgaaaacagagatgcagtcacacacatagggagggtatacaaggaaaggttaggacttcctccgaagatagtgattggttatcagtcccacgcagacacagctacaaagagcggctccaccactaaaaataggtttgttgtttaaaaagacaccttctgagtattctcacaggagactgcgtcacgcaatcgagattgggagctgaaccaaagcctcatcaaagcagagtggactgcactgaagttgattccatccaagtgttgctaagatataagagaagtctcattcgcctttgtcttgtacttctgtgttcattctcctcccccacccccaatttttgctagtgtgtccactatcccaatcaaagaattacagtatacgtcaccccagaacccgcagatgtgttcctggcccgctctgtaacagccggttagaattaccatgacacacacatttgcctttccacagtattcgaaaaagaacttgcatttctattaccttagcaggaaagatctggttttgctccactccatgcaggagcggactttgctggtgtgagagtctgagtacagctttctagcaaccttctgtttcctttcacagcattgtccttgctgtcctcttgctgatggctgctagatttaatttatttgcttccctccttgataacattagtgattctgatttcagtttttcatttgttttgcttttgtttttttcctcgtgtaacattggtgaaggatccaggaatatgacagaaaggtggaataaacattaaatttgtgcattctttggtaatttttttgtttcttgtaactacaaagctttgctacaaatttatgcatttcattcaaatcagtgatctatgtctgtgtgatccctaaacataattgtggactataaaaatgtaacaccataattacattcctaactagaattagtatgtctgcctttgtatctctatgctgtactttaacactttgtattcttaggttattttgctttggttacaatggctcaagtagaaaagcggtcccatccatattaagacagtgtacaaaactgtaaataaaatgtgtacagtgaattgtcttttagacaactagatttgtcctttatttctccatctctagaaggaatctgtacttcgtattgcaaggcagtctcttgtgtcttcttagagtgtcttccccatgcacagcctcagtttggagcactagtttattatgtttattacaatttttaataaattgactaggtagtatcacatgtaattacactgatgtggctatctttttaataaagttaaggcacagttgctcagtcctaggttgagtgatggactttgactatgttacagttgatgaggattggggttttggtgcatcaccattcggtaggaacagcggctagaaactgattgttgggtttaagatgtttttacttaatggccagaaaattagcgtaaggaaagtatatagagaaacatgcgttagggacattagtgttactatctgaataaaacacaataaacaagtattaagaactacttatattggtcaattgttgcagtatggttttctgtaaacttgaaaccttgatctattctttgtatcatttaaagcaaacatgaagacattttgtctgcagtacgtaattgtatagttcagatcctgtgagatgaggtgtggctgttaacgccgaagggtaagctgaactgtgggtagcagagtggaaaccattggctgagagaaaaatgctctttaagtggtggttgttatgaattcacactgataacttgataaagatccttataaaatacatacggaattaatagcattgctcttattatgtacgtcaagaatgtataaccgcctgctcttgttgtcacagataactccctgttcagtgctttggaaatagcgatgctcacgatctcagcattctgtaccctacatctactgtgtggatcattgagagatcttttgacattgcaacatgatatggtctatgttgggctgcattcctggctgtcttgtatgagaccccggttggctccctgaagctgattgatacagtgtacaggcatgaaggtggctgatgaggctttcttaccaacatgtgggattctagtagttgtatctattagagattaattctcatattccttttcattcatttgtaagaagtatcaactttagaagtgaaaaaagaatcataaaatacagtttttaaagtt

SEQ ID NO: 17 - DNA, 19 bp, Artificial Sequence
  PCR primer
aggacggtggctgatcaca

SEQ ID NO: 18 - DNA, 21 bp, Artificial Sequence
  PCR primer
tctctagccagaagcgatcga

SEQ ID NO: 19 - DNA, 25 bp, Artificial Sequence
  PCR probe
tgaacaagcagcagagacggagtga

SEQ ID NO: 20 - RNA, 19 nt, Artificial Sequence
  Oligomeric compound
cgagaggcggacgggaccg

SEQ ID NO: 21 - DNA, 21 bp, Artificial Sequence
  Oligomeric compound
  misc_feature (1)...(19): bases at these positions are RNA
cgagaggcggacgggaccgtt

SEQ ID NO: 22 - DNA, 21 bp, Artificial Sequence
  Oligomeric compound
  misc_feature (3)...(21): bases at these positions are RNA
ttgctctccgcctgccctggc

SEQ ID NO: 23 - RNA, 19 nt, Artificial Sequence
  Oligomeric compound
gcucuccgccugcccuggc

SEQ ID NO: 24 - RNA, 19 nt, Artificial Sequence
  Oligomeric compound
uuugaaaauguugaucucc

SEQ ID NO: 25 - RNA, 19 nt, Artificial Sequence
  Oligomeric compound
ggagaucaacauuuucaaa

SEQ ID NO: 26 - RNA, 19 nt, Artificial Sequence
  Oligomeric compound
uugucucugguccuuacuu

SEQ ID NO: 27 - RNA, 19 nt, Artificial Sequence
  Oligomeric compound
aaguaaggaccagagacaa

SEQ ID NO: 28-29 - skipped (2 entries)

SEQ ID NO: 30 - RNA, 19 nt, Artificial Sequence
  Oligomeric compound
uuuagcucuaacauuaaca

SEQ ID NO: 31 - RNA, 19 nt, Artificial Sequence
  Oligomeric compound
uguuaauguuagagcuaaa

SEQ ID NO: 32 - RNA, 19 nt, Artificial Sequence
  Oligomeric compound
uuacuagacaacuggauau

SEQ ID NO: 33 - RNA, 19 nt, Artificial Sequence
  Oligomeric compound
auauccaguugucuaguaa

SEQ ID NO: 34 - RNA, 19 nt, Artificial Sequence
  Oligomeric compound
uuaaaaagugaguagucac

SEQ ID NO: 35 - RNA, 19 nt, Artificial Sequence
  Oligomeric compound
gugacuacucacuuuuuaa

SEQ ID NO: 36 - DNA, 21 bp, Artificial Sequence
  Oligomeric compound
  misc_feature (1)...(19): bases at these positions are RNA
caaauccagaggcuagcagtt

SEQ ID NO: 37 - DNA, 21 bp, Artificial Sequence
  Oligomeric compound
  misc_feature (1)...(19): bases at these positions are RNA
cugcuagccucuggauuugtt

SEQ ID NO: 38 - RNA, 21 nt, Artificial Sequence
  Oligomeric compound
cugcuagccucuggauuuguu

SEQ ID NO: 39 - RNA, 20 nt, Artificial Sequence
  Oligomeric compound
aaguaaggaccagagacaaa

SEQ ID NO: 40 - RNA, 20 nt, Artificial Sequence
  Oligomeric compound
uuugucucugguccuuacuu

SEQ ID NO: 41 - RNA, 21 nt, Artificial Sequence
  Oligomeric compound
caaauccagaggcuagcaguu

SEQ ID NO: 42 - RNA, 20 nt, Artificial Sequence
  Oligomeric compound
cugcuagccucuggauuuga

SEQ ID NO: 43 - RNA, 17 nt, Artificial Sequence
  Oligomeric compound
gucucugguccuuacuu

SEQ ID NO: 44 - RNA, 17 nt, Artificial Sequence
  Oligomeric compound
uuuugucucugguccuu

SEQ ID NO: 45 - RNA, 17 nt, Artificial Sequence
  Oligomeric compound
cugguccuuacuucccc

SEQ ID NO: 46 - RNA, 16 nt, Artificial Sequence
  Oligomeric compound
uuugucucugguccuu

SEQ ID NO: 47 - RNA, 20 nt, Artificial Sequence
  Oligomeric compound
ucucugguccuuacuucccc

SEQ ID NO: 48 - DNA, 21 bp, Artificial Sequence
  Oligomeric compound
  misc_feature (1)...(20): bases at these positions are RNA
cugcuagccucuggauuugut

SEQ ID NO: 49 - RNA, 20 nt, Artificial Sequence
  Oligomeric compound
uucauuccuggucucuguuu

SEQ ID NO: 50 - RNA, 20 nt, Artificial Sequence
  Oligomeric compound
ugucauauuccuggauccuu

SEQ ID NO: 51 - RNA, 20 nt, Artificial Sequence
  Oligomeric compound
aaggauccaggaauaugaca

SEQ ID NO: 52 - RNA, 20 nt, Artificial Sequence
  Oligomeric compound
uccuggauccuucaccaaug

SEQ ID NO: 53 - RNA, 20 nt, Artificial Sequence
  Oligomeric compound
cauuggugaaggauccagga

SEQ ID NO: 54 - RNA, 20 nt, Artificial Sequence
  Oligomeric compound
ucuuaucaccuuuagcucua

SEQ ID NO: 55 - RNA, 20 nt, Artificial Sequence
  Oligomeric compound
uagagcuaaaggugauaaga

SEQ ID NO: 56 - RNA, 20 nt, Artificial Sequence
  Oligomeric compound
auacucagaaggugucuucu

SEQ ID NO: 57 - RNA, 20 nt, Artificial Sequence
  Oligomeric compound
agaagacaccuucugaguau

SEQ ID NO: 58 - RNA, 19 nt, Artificial Sequence
  Oligomeric compound
ucuuaucaccuuuagcucu

SEQ ID NO: 59 - RNA, 19 nt, Artificial Sequence
  Oligomeric compound
agagcuaaaggugauaaga

SEQ ID NO: 60 - RNA, 19 nt, Artificial Sequence
  Oligomeric compound
ccuggauccuucaccaaug

SEQ ID NO: 61 - DNA, 20 bp, Artificial Sequence
  Oligomeric compound
  misc_feature 1-2, 4, 6-20: bases at these positions are RNA
uutgtcucugguccuuacuu

SEQ ID NO: 62 - DNA, 20 bp, Artificial Sequence
  Oligomeric compound
  misc_feature 4, 6-20: bases at these positions are RNA
tttgtcucugguccuuacuu

SEQ ID NO: 63 - DNA, 20 bp, Artificial Sequence
  Oligomeric compound
ctgctagcctctggatttga

(19) United States
(12) Patent Application Publication
(10) Pub. No.: US 2007/0173474 A1
(43) Pub. Date: Jul. 26, 2007

(54) DOUBLE STRAND COMPOSITIONS COMPRISING DIFFERENTIALLY MODIFIED STRANDS FOR USE IN GENE MODULATION

(76) Inventors: Balkrishen Bhat, Carlsbad, CA (US); Thazha P. Prakash, Carlsbad, CA (US); Prasad Dande, Cambridge, MA (US); Charles Allerson, San Diego, CA (US); Richard H. Griffey, Vista, CA (US); Eric E. Swayze, Carlsbad, CA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/565,794

(22) Filed: Dec. 1, 2006

Related U.S. Application Data
(63) Continuation of application No. PCT/US05/19219, filed on Jun. 2, 2005.
(63) Continuation-in-part of application No. 10/859,825, filed on Jun. 3, 2004.
(63) Continuation-in-part of application No. 10/946,147, filed on Sep. 20, 2004.
(63) Continuation-in-part of application No. PCT/US04/17485, filed on Jun. 3, 2004.
(63) Continuation-in-part of application No. PCT/US04/17522, filed on Jun. 3, 2004.
(60) Provisional application No. 60/584,045, filed on Jun. 29, 2004.
(60) Provisional application No. 60/607,927, filed on Sep. 7, 2004.

(51) Int. Cl. A61K 48/00; C07H 21/02; C07F 9/6512

(52) U.S. Cl. 514/44; 514/81; 536/23.1; 544/243; 544/244

(57) ABSTRACT

The present invention provides double stranded compositions wherein each strand is modified to have a motif defined by positioning of β-D-ribonucleosides and sugar modified nucleosides. More particularly, the present compositions comprise one strand having a gapped motif and another strand having a gapped motif, a hemimer motif, a blockmer motif, a fully modified motif, a positionally modified motif or an alternating motif. At least one of the strands has complementarity to a nucleic acid target. The compositions are useful for targeting selected nucleic acid molecules and modulating the expression of one or more genes. In some embodiments, the compositions of the present invention hybridize to a portion of a target RNA resulting in loss of normal function of the target RNA. The present invention also provides methods for modulating gene expression.

### CROSS-REFERENCE TO RELATED APPLICATIONS

This application is a U.S. Continuation claiming priority to International Serial No. PCT/US2005/019219 filed Jun. 2, 2005. International Application Serial No. PCT/US2005/019219 filed Jun. 2, 2005 claims benefit to U.S. Provisional Ser. No. 60/584,045 filed Jun. 29, 2004, and U.S. Provisional Ser. No. 60/607,927 filed Sep. 7, 2004. International Application Serial No. PCT/US2005/019219 filed Jun. 2, 2005 is also a continuation-in-part of U.S. Ser. No. 10/859,825 filed Jun. 3, 2004, and U.S. Ser. No. 10/946,147 filed Sep. 20, 2004. International Application Serial No. PCT/US2005/019219 filed Jun. 2, 2005 is also a continuation-in-part of International Serial No. PCT/US2004/017485 filed Jun. 3, 2004, and International Serial No. PCT/US2004/017522 filed Jun. 3, 2004; each of which is incorporated herein by reference in its entirety.

### FIELD OF THE INVENTION

The present invention provides compositions comprising oligomeric compounds that modulate gene expression. In one embodiment, such modulation is via the RNA interference pathway. The modified oligomeric compounds of the invention comprise motifs that can enhance various physical properties and attributes compared to wild type nucleic acids. More particularly, the modification of both strands enables enhancing each strand independently for maximum efficiency for their particular roles in a selected pathway such as the RNAi pathway. The compositions are useful for, for example, targeting selected nucleic acid molecules and modulating the expression of one or more genes. In some embodiments, the compositions of the present invention hybridize to a portion of a target RNA resulting in loss of normal function of the target RNA.

### BACKGROUND OF THE INVENTION

In many species, introduction of double-stranded RNA (dsRNA) induces potent and specific gene silencing. This phenomenon occurs in both plants and animals and has roles in viral defense and transposon silencing mechanisms. This phenomenon was originally described more than a decade ago by researchers working with the petunia flower. While trying to deepen the purple color of these flowers, Jorgensen et al. introduced a pigment-producing gene under the control of a powerful promoter. Instead of the expected deep purple color, many of the flowers appeared variegated or even white. Jorgensen named the observed phenomenon “cosuppression”, since the expression of both the introduced gene and the homologous endogenous gene was suppressed (Napoli et al., Plant Cell, 1990, 2, 279-289; Jorgensen et al., Plant Mol. Biol., 1996, 31, 957-973).

Cosuppression has since been found to occur in many species of plants, fungi, and has been particularly well characterized in Neurospora crassa, where it is known as “quelling” (Cogoni et al., Genes Dev., 2000, 10, 638-643; Guru, Nature, 2000, 404, 804-808).

The first evidence that dsRNA could lead to gene silencing in animals came from work in the nematode, C. elegans. In 1995, researchers Guo and Kemphues were attempting to use antisense RNA to shut down expression of the par-1 gene in order to assess its function. As expected, injection of the antisense RNA disrupted expression of par-1, but quizzically, injection of the sense-strand control also disrupted expression (Guo et al., Cell, 1995, 81, 611-620). This result was a puzzle until Fire et al. injected dsRNA (a mixture of both sense and antisense strands) into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in first generation offspring (Fire et al., Nature, 1998, 391, 806-811).

The potency of this phenomenon led Timmons and Fire to explore the limits of the dsRNA effects by feeding nematodes bacteria that had been engineered to express dsRNA homologous to the C. elegans unc-22 gene. Surprisingly, these worms developed an unc-22 null-like phenotype (Timmons et al., Nature, 1998, 395, 854; Timmons et al., Gene, 2001, 263, 103-112). Further work showed that soaking worms in dsRNA was also able to induce silencing (Tabara et al., Science, 1998, 282, 430-431). PCT publication WO 01/48183 discloses methods of inhibiting expression of a target gene in a nematode worm involving feeding to the worm a food organism which is capable of producing a double-stranded RNA structure having a nucleotide sequence substantially identical to a portion of the target gene following ingestion of the food organism by the nematode, or by introducing a DNA capable of producing the double-stranded RNA structure.

The posttranscriptional gene silencing defined in C. elegans resulting from exposure to double-stranded RNA (dsRNA) has since been designated as RNA interference (RNAi). This term has come to generalize all forms of gene silencing involving dsRNA leading to the sequence-specific reduction of endogenous targeted mRNA levels; unlike co-suppression, in which transgenic DNA leads to silencing of both the transgene and the endogenous gene.

Introduction of exogenous double-stranded RNA (dsRNA) into C. elegans has been shown to specifically and potently disrupt the activity of genes containing homologous sequences. Montgomery et al. suggests that the primary interference effects of dsRNA are post-transcriptional; this conclusion being derived from examination of the primary DNA sequence after dsRNA-mediated interference a finding of no evidence of alterations followed by studies involving alteration of an upstream operon having no effect on the activity of its downstream gene. These results argue against an effect on initiation or elongation of transcription. Finally they observed by in situ hybridization, that dsRNA-mediated interference produced a substantial, although not complete, reduction in accumulation of nascent transcripts in the nucleus, while cytoplasmic accumulation of transcripts was virtually eliminated. These results indicate that the endogenous mRNA is the primary target for interference and suggest a mechanism that degrades the targeted mRNA before translation can occur. It was also found that this mechanism is not dependent on the SMG system, an mRNA surveillance system in C. elegans responsible for targeting and destroying aberrant messages. The authors further suggest a model of how dsRNA might function as a catalytic mechanism to target homologous mRNAs for degradation. (Montgomery et al., Proc. Natl. Acad. Sci. U S A, 1998, 95, 15502-15507).

The development of a cell-free system from syncytial blastoderm Drosophila embryos that recapitulates many of the features of RNAi has been reported. The interference observed in this reaction is sequence specific, is promoted by dsRNA but not single-stranded RNA, functions by specific mRNA degradation, and requires a minimum length of dsRNA. Furthermore, preincubation of dsRNA potentiates its activity demonstrating that RNAi can be mediated by sequence-specific processes in soluble reactions (Tuschl et al., Genes Dev., 1999, 13, 3191-3197).

In subsequent experiments, Tuschl et al, using the Drosophila in vitro system, demonstrated that 21- and 22-nt RNA fragments are the sequence-specific mediators of RNAi. These fragments, which they termed short interfering RNAs (siRNAs) were shown to be generated by an RNase III-like processing reaction from long dsRNA. They also showed that chemically synthesized siRNA duplexes with overhanging 3′ ends mediate efficient target RNA cleavage in the Drosophila lysate, and that the cleavage site is located near the center of the region spanned by the guiding siRNA. In addition, they suggest that the direction of dsRNA processing determines whether sense or antisense target RNA can be cleaved by the siRNA-protein complex (Elbashir et al., Genes Dev., 2001, 15, 188-200). Further characterization of the suppression of expression of endogenous and heterologous genes caused by the 21-23 nucleotide siRNAs have been investigated in several mammalian cell lines, including human embryonic kidney (293) and HeLa cells (Elbashir et al., Nature, 2001, 411, 494-498).

Tijsterman et al. have shown that, in fact, single-stranded RNA oligomers of antisense polarity can be potent inducers of gene silencing. As is the case for co-suppression, they showed that antisense RNAs act independently of the RNAi genes rde-1 and rde-4 but require the mutator/RNAi gene mut-7 and a putative DEAD box RNA helicase, mut-14. According to the authors, their data favor the hypothesis that gene silencing is accomplished by RNA primer extension using the mRNA as template, leading to dsRNA that is subsequently degraded suggesting that single-stranded RNA oligomers are ultimately responsible for the RNAi phenomenon (Tijsterman et al., Science, 2002, 295, 694-697).

Several other publications have described the structural requirements for the dsRNA trigger required for RNAi activity. Recent reports have indicated that ideal dsRNA sequences are 21 nt in length containing 2 nt 3′-end overhangs (Elbashir et al, EMBO (2001), 20, 6877-6887, Sabine Brantl, Biochimica et Biophysica Acta, 2002, 1575, 15-25.) In this system, substitution of the 4 nucleosides from the 3′-end with 2′-deoxynucleosides has been demonstrated to not affect activity. On the other hand, substitution with 2′-deoxynucleosides or 2′-OMe-nucleosides throughout the sequence (sense or antisense) was shown to be deleterious to RNAi activity.

Investigation of the structural requirements for RNA silencing in C. elegans has demonstrated modification of the internucleotide linkage (phosphorothioate) to not interfere with activity (Parrish et al., Molecular Cell, 2000, 6, 1077-1087.) It was also shown by Parrish et al., that chemical modification like 2′-amino or 5′-iodouridine are well tolerated in the sense strand but not the antisense strand of the dsRNA suggesting differing roles for the 2 strands in RNAi. Base modification such as guanine to inosine (where one hydrogen bond is lost) has been demonstrated to decrease RNAi activity independently of the position of the modification (sense or antisense). Same “position independent” loss of activity has been observed following the introduction of mismatches in the dsRNA trigger. Some types of modifications, for example introduction of sterically demanding bases such as 5-iodoU, have been shown to be deleterious to RNAi activity when positioned in the antisense strand, whereas modifications positioned in the sense strand were shown to be less detrimental to RNAi activity. As was the case for the 21 nt dsRNA sequences, RNA-DNA heteroduplexes did not serve as triggers for RNAi. However, dsRNA containing 2′-F-2′-deoxynucleosides appeared to be efficient in triggering RNAi response independent of the position (sense or antisense) of the 2′-F-2′-deoxynucleosides.

In one experiment the reduction of gene expression was studied using electroporated dsRNA and a 25mer morpholino in post implantation mouse embryos (Mellitzer et al., Mehanisms of Development, 2002, 118, 57-63). The morpholino oligomer did show activity but was not as effective as the dsRNA.

A number of PCT applications have been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

U.S. Pat. Nos. 5,898,031 and 6,107,094 describe certain oligonucleotide having RNA like properties. When hybridized with RNA, these olibonucleotides serve as substrates for a dsRNase enzyme with resultant cleavage of the RNA by the enzyme.

In another published paper (Martinez et al., Cell, 2002, 110, 563-574) it was shown that double stranded as well as single stranded siRNA resides in the RNA-induced silencing complex (RISC) together with elF2C1 and elf2C2 (human GERp950 Argonaute proteins. The activity of 5′-phosphorylated single stranded siRNA was comparable to the double stranded siRNA in the system studied. In a related study, the inclusion of a 5′-phosphate moiety was shown to enhance activity of siRNA's in vivo in Drosophila embryos (Boutla, et al., Curr. Biol., 2001, 11, 1776-1780). In another study, it was reported that the 5′-phosphate was required for siRNA function in human HeLa cells (Schwarz et al., Molecular Cell, 2002, 10, 537-548).

A wide variety of chemical modifications have been made to siRNA compositions to try to enhance properties including stability and potency relative to the unmodified compositions. Much of the early work looked at modification of one strand while keeping the other strand unmodified. More recent work has focused on modification of both strands.

One group is working on modifying both strands of siRNA duplexes such that each strand has an alternating pattern wherein each nucleoside or a block of modified nucleosides is alternating with unmodified β-D-ribonucleosides. The chemical modification used in the modified portion is 2′-OCH₃ modified nucleosides (see European publication EP 1389637 A1, published on Feb. 18, 2004 and PCT publication WO2004015107 published on Feb. 19, 2004).

Another group has prepared a number of siRNA constructs with modifications in both strands (see PCT publication WO03/070918 published on Aug. 28, 2003). The constructs disclosed generally have modified nucleosides dispersed in a pattern that is dictated by which strand is being modified and further by the positioning of the purines and pyrimidines in that strand. In general the purines are 2′-OCH₃ or 2′-H and pyrimidines are 2′-F in the antisense strand and the purines are 2′-H and the pyrimidines are 2′-OCH₃ or 2′-F in the sense strand. According to the definitions used in the present application these constructs would appear to be positionally modified as there is no set motif to the substitution pattern and positionally modified can describe a random substitution pattern.

Certain nucleoside compounds having bicyclic sugar moieties are known as locked nucleic acids or LNA (Koshkin et al., Tetrahedron 1998, 54, 3607-3630). These compounds are also referred to in the literature as bicyclic nucleotide analogs (Imanishi et al., International Patent Application WO 98/39352), but this term is also applicable to a genus of compounds that includes other analogs in addition to LNAs. Such modified nucleosides mimic the 3′-endo sugar conformation of native ribonucleosides with the advantage of having enhanced binding affinity and increased resistance to nucleases.

One group recently reported that the incorporation of bicyclic nucleosides, each having a 4′-CH₂—O-2′ bridge (LNA) into siRNA duplexes dramatically improved the half life in serum via enhanced nuclease resistance and also increased the duplex stability due to the increased affinity. This effect is seen with a minimum number of LNA's located as specific positions within the siRNA duplex. The placement of LNA's at the 5′-end of the sense strand was shown to reduce the loading of this strand which reduces off target effects (see Elmen et al., Nucleic Acids Res., 2005, 33(1), 439-447).

Some LNAs have a 2′-hydroxyl group linked to the 4′ carbon atom of the sugar ring thereby forming a bicyclic sugar moiety. The linkage may be a methylene (—CH₂—)ₙ group bridging the 2′ oxygen atom and the 4′ carbon atom wherein n is 1 or 2 (Singh et al., Chem. Commun., 1998, 4, 455-456; Kaneko et al., U.S. Patent Application Publication No.: US 2002/0147332, also see Japanese Patent Application HEI-11-33863, Feb. 12, 1999).

U.S. Patent Application Publication No. 2002/0068708 discloses a number of nucleosides having a variety of bicyclic sugar moieties with the various bridges creating the bicyclic sugar having a variety of configurations and chemical composition.

Braash et al., Biochemistry 2003, 42, 7967-7975 report improved thermal stability of LNA modified siRNA without compromising the efficiency of the siRNA. Grunweller, et. al., Nucleic Acid Research, 2003, 31, 3185-3193 discloses the potency of certain LNA gapmers and siRNAs.

One group has identified a 9 base sequence within an siRNA duplex that elicits a sequence-specific TLR7-dependent immune response in plasmacytoid dendritic cells. The immunostimulation was reduced by incorporating 4 bicyclic nucleosides, each having a 4′-CH₂—O-2′ bridge (LNA) at the 3′-end of the sense strand. They also made 5′ and both 3′ and 5′ versions of sense and antisense for incorporation into siRNA duplexes where one strand had the modified nucleosides and the other strand was unmodified (see Hornung et al., 2005, 11(3)1, 263-270).

One group of researchers used expression profiling to perform a genome wide analysis of the efficacy and specificity of siRNA induced silencing of two genes involved in signal transduction (insulin-like growth factor receptor (IGF1R) and mitogen-activated protein kinase 1 (MAPK14 or p38α). A unique expression profile was produced for each of the 8 siRNAs targeted to MAPK14 and 16 siRNA's targeted to IGF1R indicating that off target effects were highly dependent on the particular sequence. These expression patterns were reproducable for each individual siRNA. The group determined that off target effects were caused by both the antisense strand and the sense strand of siRNA duplexes. There is a need for siRNA's that are designed to preferentially load only the antisense strand thereby reducing the off target effects caused by the sense strand also being loaded into the RISC.

A number of published applications that are commonly assigned with the present application disclose double strand compositions wherein one or both of the strands comprise a particular motif. The motifs include hemimer motifs, blockmer motifs, gapped motifs, fully modified motifs, positionally modified motifs and alternating motifs (see published PCT applications: WO 2004/044133 published May 27, 2004, 3′-endo motifs; WO 2004/113496 published Dec. 29, 2004, 3′-endo motifs; WO 2004/044136 published May 27, 2004, alternating motifs; WO 2004/044140 published May 27, 2004, 2′-modified motifs; WO 2004/043977 published May 27, 2004, 2′-F motifs; WO 2004/043978 published May 27, 2004, 2′-OCH₃ motifs; WO 2004/041889 published May 21, 2004, polycyclic sugar motifs; WO 2004/043979 published May 27, 2004, sugar surrogate motifs; and WO 2004/044138 published May 27, 2004, chimeric motifs; also see published US Application US20050080246 published Apr. 14, 2005).

Like the RNAse H pathway, the RNA interference pathway of antisense modulation of gene expression is an effective means for modulating the levels of specific gene products and may therefore prove to be uniquely useful in a number of therapeutic, diagnostic, and research applications involving gene silencing. The present invention therefore further provides compositions useful for modulating gene expression pathways, including those relying on an antisense mechanism of action such as RNA interference and dsRNA enzymes as well as non-antisense mechanisms. One having skill in the art, once armed with this disclosure will be able, without undue experimentation, to identify additional compositions for these uses.

### SUMMARY OF THE INVENTION

In one embodiment, the present invention provides compositions comprising a first oligomeric compound and a second oligomeric compound wherein at least a portion of the first oligomeric compound is capable of hybridizing with at least a portion of the second oligomeric compound and at least a portion of the first oligomeric compound is complementary to and capable of hybridizing to a selected nucleic acid target. One of the first and second oligomeric compounds comprises nucleosides linked by internucleoside linking groups wherein the linked nucleosides comprise a gapped motif. The other of the first and second oligomeric compounds comprises nucleosides linked by internucleoside linking groups wherein the linked nucleosides comprise a gapped motif, an alternating motif, a positionally modified motif, a fully modified motif, a blockmer motif or a hemimer motif.

The compositions further comprise one or more optional overhangings, phosphate moieties, conjugate groups or capping groups. When the first and second oligomeric compounds each independently comprise gapped motifs then at least one of the 3′ or 5′ termini of at least one of the first and second oligomeric compounds comprises modified nuleosides other than 2′-OCH₃ modified nucleosides or at least one of the first and second oligomeric compounds comprises an asymmetric gapped motif.

In one embodiment, each oligomeric compound comprising a gapped motif comprises an internal region of linked nucleosides flanked by two external regions of linked nucleosides wherein the nucleosides of the internal region are different from the nucleosides of each of the external regions and wherein the nucleosides of each of the external regions are independently selected from 2′-modified nucleosides, 4′-thio modified nucleosides, 4′-thio-2′-modified nucleosides and nucleosides having bicyclic sugar moieties. In one embodiment, the internal region of at least one of the oligomeric compounds having a gapped motif is a sequence of β-D-ribonucleosides. In another embodiment, the internal region of at least one of the oligomeric compounds having a gapped motif is a sequence of modified nucleosides with 2′-F or 4′-thio modified nucleosides.

In one embodiment, one of the first and second oligomeric compounds comprises a symmetric gapped motif. In another embodiment, at least one of the first and second oligomeric compounds comprises an asymmetric gapped motif. In a further embodiment, one of the first and second oligomeric compounds comprises a symmetric gapped motif and the other of the first and second oligomeric compounds comprises an asymmetric gapped motif.

In another embodiment, at least one of the external regions of at least one of the first and second oligomeric compounds comprises 2′-modified nucleosides. In a further embodiment, each of the external regions of at least one of the first and second oligomeric compounds comprises 2′-modified nucleosides.

In one embodiment, at least one of the external regions of at least one of the oligomeric compounds is modified with 2′-modified nucleosides wherein each of the 2′-modifications is, independently, halo, allyl, amino, azido, O-allyl, O—C₁₋₁₀alkyl, OCF₃, O—(CH₂)₂—O—CH₃, 2′-O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. 2′-modifications include —F, —OCH₃ or —O—(CH₂)₂—O—CH₃.

In one embodiment, at least one of the external regions of at least one of the first and second oligomeric compounds comprises 4′-thio modified nucleosides. In another embodiment, at least one of the external regions of at least one of the first and second oligomeric compounds comprises 4′-thio-2′-modified nucleosides. In one embodiment, the 2′-substituent groups of the 4′-thio-2′-modified nucleosides are selected from halogen, allyl, amino, azido, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. In one embodiment, each of the 2′-substituent groups of the 4′-thio-2′-modified nucleosides are selected from —F, —OCH₃, —OCF₃ or —O—(CH₂)₂—O—CH₃ with —OCH₃ or —O—(CH₂)₂—O—CH₃ being suitable.

In one embodiment, at least one of the external regions of at least one of the first and second oligomeric compounds comprises bicyclic sugar moieties. In another embodiment, each of the bicyclic sugar moieties independently, comprises a 2′-O—(CH₂)ₙ-4′ bridge wherein n is 1 or 2.

In one embodiment, the first oligomeric compound comprises a gapped motif. In a further embodiment, the first oligomeric compound comprises a gapped motif wherein each of the external regions independently comprises 4′-thio modified nucleosides or 2′-modified nucleosides. In another embodiment, one of the external regions of the first oligomeric compound comprises 4′-thio modified nucleosides and the other external region comprises 2′-modified nucleosides. In another embodiment, the 2′-modified nucleosides are 2′-OCH₃ or 2′-F modified nucleosides with 2′-OCH₃ modified nucleosides are suitable. In another embodiment, the external region located at the 5′-end of the first oligomeric compound comprises 2′-OCH₃, 2′-F or 4′-thio modified nucleosides.

In one embodiment, the second oligomeric compound comprises a gapped motif. In another embodiment, the external regions of the gapped second oligomeric compound comprise 2′-modified nucleosides, 4′-thio modified nucleosides, 4′-thio-2′-modified nucleosides or nucleosides having bicyclic sugar moieties. In a further embodiment, at least one of the external regions of the gapped second oligomeric compound comprise 2′-modified nucleosides selected from halogen, allyl, amino, azido, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. In another embodiment at least one of the external regions of the second gapped oligomeric compound comprise 2′-modified nucleosides selected from allyl, O-allyl, O—C₂-C₁₀ alkyl, O—(CH₂)₂—O—CH₃ or 2′-O(CH₂)₂SCH₃. In another embodiment each of the 2′-modified nucleosides of the second gapped oligomeric compound is a 2′-O—(CH₂)₂—O—CH₃ modified nucleoside.

In another embodiment, at least one of the external regions of at least one of the first and second oligomeric compounds comprises at least one bicyclic sugar moiety. Each of the modified sugars in one of the external regions can be a bicyclic sugar moiety. Bicyclic sugar moieties independently, comprises a 2′-O—(CH₂)ₙ-4′ bridge wherein n is 1 or 2.

In one embodiment, the external regions of each of the oligomeric compounds comprising a gapped motif each independently comprise from about 1 to about 6 nucleosides. In another embodiment, each of the oligomeric compounds comprising a gapped motif each independently comprise from about 1 to about 4 nucleosides. In another embodiment, each of the oligomeric compounds comprising a gapped motif each independently comprise from about 1 to about 3 nucleosides.

In one embodiment, one of the first and second oligomeric compounds comprises an alternating motif having the formula:
5′-A(-L-B-L-A)ₙ(-L-B)ₙₙ-3′
wherein:

each L is, independently, an internucleoside linking group;

each A is a β-D-ribonucleoside or a sugar modified nucleoside;

each B is a β-D-ribonucleoside or a sugar modified nucleoside;

n is from about 7 to about 11;

nn is 0 or 1; and

wherein the sugar groups comprising each A nucleoside are identical, the sugar groups comprising each B nucleoside are identical, the sugar groups of the A nucleosides are different than the sugar groups of the B nucleosides and at least one of A and B is a sugar modified nucleoside.

In one embodiment, each A or each B is a β-D-ribonucleoside. In another embodiment, each A or each B is a 2′-modified nucleoside wherein the 2′-substituent is selected from halogen, allyl, amino, azido, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ) or O—CH₂—C(═O)—N(Rₘ)(Rₙ), where each Rₘ and R, is, independently, H, an amino protecting group or substituted or unsubstituted C₁-C₁₀ alkyl. In one embodiment the 2′-substituent is allyl, O-allyl, O—C₁-C₁₀ alkyl, O—(CH₂)₂—O—CH₃ or 2′-O(CH₂)₂SCH₃ with O—(CH₂)₂—O—CH₃ being particularly suitable.

In one embodiment each A and each B is modified nucleoside. In one embodiment, one of each A and each B comprises 2′-OCH₃ modified nucleosides. In another embodiment, each A and each B comprises 2′-F modified nucleosides.

In one embodiment, the second oligomeric compound comprises an alternating motif and one of each A and each B are β-D-ribonucleosides. In another embodiment, the other of each A and each B comprises 2′-modified nucleosides wherein suitable 2′-substituents include, but are not limited to, allyl, O-allyl, O—C₁-C₁₀alkyl, O—(CH₂)₂—O—CH₃ or 2′-O(CH₂)₂SCH₃ with O—(CH₂)₂—O—CH₃ being particularly suitable.

In one embodiment, each L is independently a phosphodiester or a phosphorothioate internucleoside linking group.

In one embodiment, one of the first and the second oligomeric compounds comprises a fully modified motif wherein essentially each nucleoside of the oligomeric compound is a sugar modified nucleoside and wherein each sugar modification is the same. In one embodiment, each sugar modified nucleoside is selected from 2′-modified nucleosides, 4′-thio modified nucleosides, 4′-thio-2′-modified nucleosides and nucleosides having bicyclic sugar moieties. In another embodiment, each nucleoside of the fully modified oligomeric compound is a 2′-modified nucleoside wherein 2′-OCH₃ or a 2′-F modified nucleosides are suitable and 2′-OCH₃ modified nucleosides are particularly suitable. In another embodiment, the fully modified oligoeric compound includes one or both of the 3′ and 5′-termini having one β-D-ribonucleoside.

In one embodiment, one of the first and second oligomeric compounds comprises a positionally modified wherein the positionally modified motif comprises a continuous sequence of linked nucleosides comprising from about 4 to about 8 regions wherein each region is either a sequence of β-D-ribonucleosides or a sequence of sugar modified nucleosides and wherein the regions are alternating wherein each of the β-D-ribonucleoside regions is flanked on each side by a region of sugar modified nucleosides and each region of sugar modified nucleosides is flanked on each side by a β-D-ribonucleoside region with the exception of regions located the 3′ and 5′-termini that will only be flanked on one side and wherein the sugar modified nucleosides are selected from 2′-modified nucleosides, 4′-thio modified nucleosides, 4′-thio-2′-modified nucleosides and nucleosides having bicyclic sugar moieties. In one embodiment, the positionally modified motif comprises from 5 to 7 regions. In another embodiment, the regions of β-D-ribonucleosides comprise from 2 to 8 nucleosides in length. In a further embodiment, the regions of sugar modified nucleosides comprises from 1 to 4 nucleosides in length or from 2 to 3 nucleosides in length.

In one embodiment, oligomeric compounds comprising a positionally modified motif have the formula:
(X₁)ⱼ-(Y₁)ᵢ-X₂-Y₂-X₃-Y₃-X₄
wherein:

X₁ is a sequence of from 1 to about 3 sugar modified nucleosides;

Y₁ is a sequence of from 1 to about 5 β-D-ribonucleosides;

X₂ is a sequence of from 1 to about 3 sugar modified nucleosides;

Y₂ is a sequence of from 2 to about 7 β-D-ribonucleosides;

X₃ is a sequence of from 1 to about 3 sugar modified nucleosides;

Y₃ is a sequence of from 4 to about 6 β-D-ribonucleosides;

X₄ is a sequence of from 1 to about 3 sugar modified nucleosides;

i is 0 or 1; and

j is 0 or 1 when i is 1 or 0 when i is 0.

In another embodiment, X₄ is a sequence of 3 sugar modified nucleosides, Y₃ is a sequence of 5 β-D-ribonucleosides, X₃ is a sequence of 2 sugar modified nucleosides; and Y₁ is a sequence of 2 β-D-ribonucleosides. In another embodiment i is 0 and Y₂ is a sequence of 7 β-D-ribonucleosides. In another embodiment i is 1, j is 0, Y₂ is a sequence of 2 β-D-ribonucleosides and Y₁ is a sequence of 5 β-D-ribonucleosides. In another embodiment i is 1, j is 1, Y₂ is a sequence of 2 β-D-ribonucleosides, Y₁ is a sequence of 3 β-D-ribonucleosides and X₁ is a sequence of 2 sugar modified nucleosides. In one embodiment, each of the sugar modified nucleosides is a 2′-modified nucleoside or a 4′-thio modified nucleoside.

In one embodiment, the first strand of the composition comprises the positional motif. In another embodiment, each internucleoside linking group of the positionally modified oligomeric compound is independently selected from phosphodiester or phosphorothioate.

In one embodiment, each of the first and second oligomeric compounds independently comprises from about 12 to about 30 nucleosides. In a further embodiment, each of the first and second oligomeric compounds independently comprises from about 17 to about 23 nucleosides. In another embodiment, each of the first and second oligomeric compounds independently comprises from about 19 to about 21 nucleosides.

In one embodiment, the first and the second oligomeric compounds form a complementary antisense/sense siRNA duplex.

In one embodiment, the present invention also provides methods of inhibiting gene expression comprising contacting one or more cells, a tissue or an animal with a composition described herein.

In another embodiment, compositions of the invention are used in the preparation of medicaments for inhibiting gene expression in a cell, tissue or animal.

### DESCRIPTION OF EMBODIMENTS

The present invention provides double stranded compositions wherein each strand comprises a motif defined by the location of one or more modified nucleosides or modified and unmodified nucleosides. Motifs derive from the positioning of modified nucleosides relative to other modified or unmodified nucleosides in a strand and are independent of the type of internucleoside linkage, the nucleobase or type of nucleobase e.g. purines or pyrimidines. The compositions of the present invention comprise strands that are differentially modified so that either the motifs or the chemistry of each are different. This strategy allows for maximizing the desired properties of each strand independently for their intended role in a process of gene modulation e.g. RNA interference. Tailoring the chemistry and the motif of each strand independently also allows for regionally enhancing each strand. More particularly, the present compositions comprise one strand having a gapped motif and another strand having a gapped motif, a hemimer motif, a blockmer motif, a fully modified motif, a positionally modified motif or an alternating motif.

The compositions comprising the various motif combinations of the present invention have been shown to have enhanced properties. The properties that can be enhanced include, but are not limited, to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage.

Compositions are provided comprising a first and a second oligomeric compound that are fully or at least partially hybridized to form a duplex region and further comprising a region that is complementary to and hybridizes to a nucleic acid target. It is suitable that such a composition comprise a first oligomeric compound that is an antisense strand having full or partial complementarity to a nucleic acid target and a second oligomeric compound that is a sense strand having one or more regions of complementarity to and forming at least one duplex region with the first oligomeric compound.

The compositions of the present invention are useful for, for example, modulating gene expression. For example, a targeted cell, group of cells, a tissue or an animal is contacted with a composition of the invention to effect reduction of mRNA that can directly inhibit gene expression. In another embodiment, the reduction of mRNA indirectly upregulates a non-targeted gene through a pathway that relates the targeted gene to a non-targeted gene. Numerous methods and models for the regulation of genes using compositions of the invention are illustrated in the art and in the example section below.

The compositions of the invention modulate gene expression by hybridizing to a nucleic acid target resulting in loss of its normal function. As used herein, the term “target nucleic acid” or “nucleic acid target” is used for convenience to encompass any nucleic acid capable of being targeted including without limitation DNA, RNA (including pre-mRNA and mRNA or portions thereof) transcribed from such DNA, and also cDNA derived from such RNA. In some embodiments, the target nucleic acid is a messenger RNA. In another embodiment, the degradation of the targeted messenger RNA is facilitated by an activated RISC complex that is formed with compositions of the invention. In another embodiment, the degradation of the targeted messenger RNA is facilitated by a nuclease such as RNaseH.

The present invention provides double stranded compositions wherein one of the strands is useful in, for example, influencing the preferential loading of the opposite strand into the RISC (or cleavage) complex. In particular, the present invention provides oligomeric compounds that comprise chemical modifications in at least one of the strands to drive loading of the opposite strand into the RISC (or cleavage) complex. Such modifications can be used to increase potency of duplex constructs that have been modified to enhance stability. Examples of chemical modifications that drive loading of the second strand are expected to include, but are not limited to, MOE (2′-O(CH₂)₂OCH₃), 2′-O-methyl, -ethyl, -propyl, and —N-methylacetamide. Such modifications can be distributed throughout the strand, or placed at the 5′ and/or 3′ ends to make a gapmer motif on the sense strand. The compositions are useful for targeting selected nucleic acid molecules and modulating the expression of one or more genes. In some embodiments, the compositions of the present invention hybridize to a portion of a target RNA resulting in loss of normal function of the target RNA.

The present invention provides double stranded compositions wherein one strand comprises a gapped motif and the other strand comprises a gapped motif, a hemimer motif, a blockmer motif, a fully modified motif, a positionally modified motif or an alternating motif. Each strand of the compositions of the present invention can be modified to fulfil a particular role in for example the siRNA pathway. Using a different motif in each strand or the same motif with different chemical modifications in each strand permits targeting the antisense strand for the RISC complex while inhibiting the incorporation of the sense strand. Within this model each strand can be independently modified such that it is enhanced for its particular role. The antisense strand can be modified at the 5′-end to enhance its role in one region of the RISC while the 3′-end can be modified differentially to enhance its role in a different region of the RISC. Researchers have been looking at the interaction of the guide sequence and the RISC using various models. Different requirements for the 3′-end, the 5′-end and the region corresponding to the cleavage site of the mRNA are being elucidated through these studies. It has now been shown that the 3′-end of the guide sequence complexes with the PAZ domain while the 5′-end complexes with the Piwi domain (see Song et al., Science, 2004, 305, 1434-1437; Song et al., Nature Structural Biology, 2003, 10(12), 1026-1032; Parker et al., Letters to Nature, 2005, 434, 663-666).

As used in the present invention the term “gapped motif” is meant to include a contiguous sequence of nucleosides that are divided into 3 regions, an internal region flanked by two external regions. The regions are differentiated from each other at least by having different sugar groups that comprise the nucleosides. The types of nucleosides that are used to differentiate the regions of a gapped oligomeric compound include β-D-ribonucleosides, 2′-modified nucleosides, 4′-thio modified nucleosides, 4′-thio-2′-modified nucleosides, and bicyclic sugar modified nucleosides. Each region is uniformly modified e.g. the sugar groups are identical. The internal region or the gap generally comprises β-D-ribonucleosides but can be a sequence of sugar modified nucleosides. The nucleosides located in the gap of a gapped oligomeric compound have different sugar groups than both of the wings.

Gapped oligomeric compounds are further defined as being either “symmetric” or “asymmetric”. A gapmer having the same uniform sugar modification in each of the wings is termer a symmetric gapped oligomeric compound. A gapmer having different uniform modifications in each wing is termed an asymmetric gapped oligomeric compound. Gapped oligomeric compounds such as these can have for example both wings comprising 4′-thio modified nucleosides (symmetric gapmer) and a gap comprising β-D-ribonucleosides or modified nucleosides other than 4′-thio modified nucleosides. Asymmetric gapped oligomeric compounds for example can have one wing comprising 2′-OCH₃ modified nucleosides and the other wing comprising 4′-thio modified nucleosides with the internal region (gap) comprising β-D-ribonucleosides or sugar modified nucleosides that are other than 4′-thio or 2′-OCH₃ modified nucleosides.

Gapped oligomeric compounds as used in the present invention include wings that independently have from 1 to about 6 nucleosides. Suitable wings comprise from 1 to about 4 nucleosides and can comprise wings comprising from 1 to about 3 nucleosides. The number of nucleosides in each wing can be the same or different. The present invention therefore includes gapped oligomeric compounds wherein each wing independently comprises 1, 2, 3, 4, 5, or 6 sugar modified nucleosides.

Gapped oligomeric compounds can be chemically modified to enhance their properties and differential modifications can be made to specifically enhance the antisense strand or the sense strand of an siRNA duplex. In one embodiment of the present invention both strands are gapped oligomeric compounds. When both strands are gapped oligomeric compounds at least one is an asymmetric gapped oligomeric compound or at least one of the wings of one of the gapped oligomeric compounds comprises sugar modified nucleosides that are other than 2′-OCH₃ modified nucleosides.

Oligomeric compounds of the invention comprising a gapped motif in each strand generally utilize sugar modifications in the wings of each strand that will enhance that strand for its intended role in gene modulation. For example using 2′-MOE (2′-O—(CH₂)₂—OCH₃) modifications in the wings of the sense strand increases the efficiency of the antisense strand. It is believed that the bulky wings of a MOE gapmer inhibits its incorporation into the RISC complex thereby allowing preferential loading of the antisense strand resulting in a reduction of off target effects and increased potency of the antisense strand. LNA modified nucleosides have also been used to inhibit the uptake of the sense strand in compositions of the invention.

The gapped oligomeric compound that has been modified for use as the sense strand can be paired with a gapped oligomeric compound that is specifically modified for use as the antisense strand. The antisense strand can comprise sugar modified nucleosides in the wings that do not inhibit incorporation into the RISC and that will further enhance other properties such as nuclease stability. A number of gapped compositions were made and tested wherein the wings of the antisense strand had sugar modifications selected from 2′-F, 2′-OCH₃ and 4′-thio. These antisense strands were prepared with both symmetric and asymmetric motifs. The asymmetric motif when used for the antisense strand further allowed matching the different chemistries of the 3′ and the 5′-ends to the functionally different roles each fulfils within the RISC complex. A number of different asymmetric gapped antisense strands were made and were paired with different sense strands to determine their activities (activity data shown in the example section below).

As used in the present invention the term “alternating motif” is meant to include a contiguous sequence of nucleosides comprising two different nucleosides that alternate for essentially the entire sequence of the oligomeric compound. The pattern of alternation can be described by the formula: 5′-A(-L-B-L-A)ₙ(-L-B)ₙₙ-3′ where A and B are nucleosides differentiated by having at least different sugar groups, each L is an internucleoside linking group, nn is 0 or 1 and n is from about 7 to about 11. This permits alternating oligomeric compounds from about 17 to about 24 nucleosides in length. This length range is not meant to be limiting as longer and shorter oligomeric compounds are also amenable to the present invention. This formula also allows for even and odd lengths for alternating oligomeric compounds wherein the 3′ and 5′-terminal nucleosides are the same (odd) or different (even).

The “A” and “B” nucleosides comprising alternating oligomeric compounds of the present invention are differentiated from each other by having at least different sugar moieties. Each of the A and B nucleosides is selected from β-D-ribonucleosides, 2′-modified nucleosides, 4′-thio modified nucleosides, 4′-thio-2′-modified nucleosides, and bicyclic sugar modified nucleosides. The alternating motif includes the alternation of nucleosides having different sugar groups but is independent from the nucleobase sequence and the internucleoside linkages. The internucleoside linkage can vary at each or selected locations or can be uniform or alternating throughout the oligomeric compound.

Alternating oligomeric compounds of the present invention can be designed to function as the sense or the antisense strand. Alternating 2′-OCH₃/2′-F modified oligomeric compounds have been used as the antisense strand and have shown good activity with a variety of sense strands. One antisense oligomeric compound comprising an alternating motif is a 19mer wherein the A's are 2′-OCH₃ modified nucleosides and the B's are 2′-F modified nucleosides (nn is 0 and n is 9). The resulting alternating oligomeric compound will have a register wherein the 3′ and 5′-ends are both 2′-OCH₃ modified nucleosides.

Alternating oligomeric compounds have been designed to function as the sense strand also. The chemistry or register is generally different than for the oligomeric compounds designed for the antisense strand. When a alternating 2′-F/2′-OCH₃ modified 19mer was paired with the antisense strand in the previous paragraph the preferred orientation was determined to be an offset register wherein both the 3′ and 5′-ends of the sense strand were 2′-F modified nucleosides. In a matched register the sugar modifications match between hybridized nucleosides so all the terminal ends of an 19mer would have the same sugar modification. Another alternating motif that has been tested and works in the sense strand is β-D-ribonucleosides alternating with 2′-MOE modified nucleosides.

As used in the present invention the term “fully modified motif” is meant to include a contiguous sequence of sugar modified nucleosides wherein essentially each nucleoside is modified to have the same sugar modification. The compositions of the invention can comprise a fully modified strand as the sense or the antisense strand with the sense strand preferred as the fully modified strand. Suitable sugar modified nucleosides for fully modified strands of the invention include 2′-F, 4′-thio and 2′-OCH₃ with 2′-OCH₃ particularly suitable. In one aspect the 3′ and 5′-terminal nucleosides are unmodified.

As used in the present invention the term “hemimer motif” is meant to include a sequence of nucleosides that have uniform sugar moieties (identical sugars, modified or unmodified) and wherein one of the 5′-end or the 3′-end has a sequence of from 2 to 12 nucleosides that are sugar modified nucleosides that are different from the other nucleosides in the hemimer modified oligomeric compound. An example of a typical hemimer is a an oligomeric compound comprising β-D-ribonucleosides that have a sequence of sugar modified nucleosides at one of the termini. One hemimer motif includes a sequence of β-D-ribonucleosides having from 2-12 sugar modified nucleosides located at one of the termini. Another hemimer motif includes a sequence of β-D-ribonucleosides having from 2-6 sugar modified nucleosides located at one of the termini with from 2-4 being suitable.

As used in the present invention the term “blockmer motif” is meant to include a sequence of nucleosides that have uniform sugars (identical sugars, modified or unmodified) that is internally interrupted by a block of sugar modified nucleosides that are uniformly modified and wherein the modification is different from the other nucleosides. More generally, oligomeric compounds having a blockmer motif comprise a sequence of β-D-ribonucleosides having one internal block of from 2 to 6, or from 2 to 4 sugar modified nucleosides. The internal block region can be at any position within the oligomeric compound as long as it is not at one of the termini which would then make it a hemimer. The base sequence and internucleoside linkages can vary at any position within a blockmer motif.

As used in the present invention the term “positionally modified motif” is meant to include a sequence of β-D-ribonucleosides wherein the sequence is interrupted by two or more regions comprising from 1 to about 4 sugar modified nucleosides. The positionally modified motif includes internal regions of sugar modified nucleoside and can also include one or both termini. Each particular sugar modification within a region of sugar modified nucleosides is variable with uniform modification desired. The sugar modified regions can have the same sugar modification or can vary such that one region may have a different sugar modification than another region. Positionally modified strands comprise at least two sugar modified regions and at least three when both the 3′ and 5′-termini comprise sugar modified regions. Positionally modified oligomeric compounds are distinguished from gapped motifs, hemimer motifs, blockmer motifs and alternating motifs because the pattern of regional substitution defined by any positional motif is not defined by these other motifs. Positionally modified motifs are not determined by the nucleobase sequence or the location or types of internucleoside linkages. The term positionally modified oligomeric compound includes many different specific substitution patterns. A number of these substitution patterns have been prepared and tested in compositions.

Either the antisense or the sense strand of compositions of the present invention can be positionally modified. In one embodiment, the positionally modified strand is designed as the antisense strand. A list of different substitution patterns corresponding to positionally modified oligomeric compounds illustrated in the examples are shown below. This list is meant to be instructive and not limiting.

| | | Substitution pattern 5′-3′ | Modified positions |
|---|---|---|---|
| ISIS No: | Length | underlined are modified | from 5′-end |
| 345838 | 19mer | 5-1-5-1-2-1-2-2 | 6, 12, 15 and 18-19 |
| 352506 | 19mer | 5-2-2-2-5-3 | 7-8, 10-11, 17-19 |
| 352505 | 19mer | 4-1-2-1-2-1-2-1-2-3 | 5, 8, 11, 14, 17-19 |
| xxxxxx | 19mer | 4-1-6-1-4-3 | 5, 12, 17-19 |
| xxxxxx | 19mer | 4-2-4-2-5-2 | 5-6, 11-12, 18-19 |
| 345839 | 19mer | 4-2-2-2-6-3 | 5-6, 9-10, 17-19 |
| xxxxxx | 19mer | 3-1-4-1-4-1-3-1-1 | 4, 9, 14, 18 |
| 353539 | 19mer | 3-5-1-2-1-4-3 * | 1-3, 9, 12 |
| 355715 | 19mer | 3-1-4-1-8-1-1 | 4, 9, 18 |
| xxxxxx | 19mer | 3-1-5-1-7-1-1 | 4, 10, 18 |
| 384760 | 19mer | 2-7-2-5-3 * | 1-2, 10-11 and 17-19 |
| 371315 | 19mer | 3-6-2-5-3 | 1-3, 10-11, 17-19 |
| 353538 | 19mer | 2-1-5-1-2-1-4-3 | 3, 9, 12, 17-19 |
| xxxxxx | 19mer | 2-1-4-1-4-1-4-1-1 | 3, 8, 13, 18 |
| 336674 | 20mer | 15-1-1-3 | 16, 18-20 |
| 355712 | 20mer | 4-1-2-1-2-1-2-1-2-3 * | 5, 8, 11, 14 |
| 347348 | 20mer | 3-2-1-2-1-2-1-2-1-2-3 | 1-3, 6, 9, 12, 15, 18-20 |
| 348467 | 20mer | 3-2-1-2-1-2-1-2-1-5 | 1-3, 6, 9, 12, 15 |
| 357278 | 20mer | 3-1-4-1-4-1-3-1-1 | 4, 9, 14, 18 |
| xxxxxx | 20mer | 3-1-1-10-1-1-3 | 1-3, 5, 16, 18-20 |
| xxxxxx | 20mer | 3-1-6-1-7-1-1 | 4, 11, 19 |
| 357276 | 20mer | 3-1-3-1-7-1-4 | 4, 8, 16 |
| xxxxxx | 20mer | 3-1-5-2-5-1-3 | 4, 11, 17 |
| 357275 | 20mer | 3-1-5-1-8-1-1 | 4, 10, 19 |
| 373424 | 20mer | 3-6-2-5-3 | 1-3, 11-12, 18-20 |
| 357277 | 20mer | 2-1-5-1-5-1-4-2 | 3, 9, 15, 20-21 |
| 345712 | 20mer | 2-2-5-2-5-2-2 | 3-4, 10-11, 17-18 |

* indicates that more than one type of sugar modified nucleosides were used in the sugar modified regions.

The term “sugar modified nucleosides” as used in the present invention is intended to include all manner of sugar modifications known in the art. The sugar modified nucleosides can have any heterocyclic base moiety and internucleoside linkage and may include further groups independent from the sugar modification. A group of sugar modified nucleosides includes 2′-modified nucleosides, 4′-thio modified nucleosides, 4′-thio-2′-modified nucleosides, and bicyclic sugar modified nucleosides.

The term “2′-modified nucleoside” as used in the present invention is intended to include all manner of nucleosides having a 2′-substituent group that is other than H and OH. Suitable 2′-substituent groups for 2′-modified nucleosides of the invention include, but are not limited to: halo, allyl, amino, azido, amino, SH, CN, OCN, CF₃, OCF₃, O—, S—, or N(Rₘ)-alkyl; O—, S—, or N(Rₘ)-alkenyl; O—, S— or N(Rₘ)-alkynyl; O-alkylenyl-O-alkyl, alkynyl, alkaryl, aralkyl, O-alkaryl, O-aralkyl, O(CH₂)₂SCH₃, 0-(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with substituent groups selected from hydroxyl, amino, alkoxy, carboxy, benzyl, phenyl, nitro (NO₂), thiol, thioalkoxy (S-alkyl), halogen, alkyl, aryl, alkenyl and alkynyl where each Rₘ is, independently, H, an amino protecting group or substituted or unsubstituted C₁-C₁₀ alkyl.

A list of 2′-substituent groups includes F, —NH₂, N₃, OCF₃, O—CH₃, O(CH₂)₃NH₂), CH₂—CH═CH₂, —O—CH₂—CH═CH₂, OCH₂CH₂OCH₃, 2′-O(CH₂)₂SCH₃, O—(CH₂)₂—O—N(Rₘ)(Rₙ), —O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl. Another list of 2′-substituent groups includes F, OCF₃, O—CH₃, OCH₂CH₂OCH₃, 2′-O(CH₂)₂SCH₃, O—(CH₂)₂—O—N(Rₘ)(Rₙ), —O(CH₂)₂O(CH₂)₂N(CH₃)₂, and N-substituted acetamides (O—CH₂—C(═O)—N(Rₘ)(Rₙ) where each Rₘ and Rₙ is, independently, H, an amino protecting group or substituted or unsubstituted C₁-C₁₀ alkyl.

Also amenable to the present invention is the manipulation of the stereochemistry of the basic furanose ring system which can be prepared in a number of different configurations. The attachment of the heterocyclic base to the 1′-position can result in the α-anomer (down) or the β-anomer (up). The β-anomer is the anomer found in native DNA and RNA but both forms can be used to prepare oligomeric compounds. A further manipulation can be achieved through the substitution the native form of the furanose with the enantiomeric form e.g. replacement of a native D-furanose with its mirror image enantiomer, the L-furanose. Another way to manipulate the furanose ring system is to prepare stereoisomers such as for example substitution at the 2′-position to give either the ribofuranose (down) or the arabinofuranose (up) or substitution at the 3′-position to give the xylofuranose or by altering the 2′, and the 3′-position simultaneously to give a xylofuranose. The use of stereoisomers of the same substituent can give rise to completely different conformational geometry such as for example 2′-F which is 3′-endo in the ribo configuration and 2′-endo in the arabino configuration. The use of different anomeric and stereoisomeric sugars in oligomeric compounds is known in the art and amenable to the present invention.

The term “4′-thio modified nucleoside” is intended to include β-D-ribonucleosides having the 4′-O replaced with 4′-S. The term “4′-thio-2′-modified nucleoside” is intended to include 4′-thio modified nucleosides having the 2′-OH replaced with a 2′-substituent group. The preparation of 4′-thio modified nucleosides is disclosed in publications such as for example U.S. Pat. No. 5,639,837 issued Jun. 17, 1997 and PCT publication WO 2005/027962 published on Mar. 31, 2005. The preparation of 4′-thio-2′-modified nucleosides and their incorporation into oligonucleotides is disclosed in the PCT publication WO 2005/027962 published on Mar. 31, 2005. The 4′-thio-2′-modified nucleosides can be prepared with the same 2′-substituent groups previously mentioned with 2′-OCH₃, 2′-O—(CH₂)₂—OCH₃ and 2′-F are suitable groups.

The term “bicyclic sugar modified nucleoside” is intended to include nucleosides having a second ring formed from the bridging of 2 atoms of the ribose ring. Such bicyclic sugar modified nucleosides can incorporate a number of different bridging groups that form the second ring and can be formed from different ring carbon atoms on the furanose ring. Bicyclic sugar modified nucleosides wherein the bridge links the 4′ and the 2′-carbons and has the formula 4′-(CH₂), —O-2′ wherein n is 1 or 2 are suitable. The synthesis of bicyclic sugar modified nucleosides is disclosed in U.S. Pat. Nos. 6,268,490, 6,794,499 and published U.S. application 20020147332.

The synthesis and preparation of the bicyclic sugar modified nucleosides wherein the bridge is 4′-CH₂—O-2′ having nucleobases selected from adenine, cytosine, guanine, 5-methyl-cytosine, thymine and uracil, along with their oligomerization, and nucleic acid recognition properties have been described (Koshkin et al., Tetrahedron, 1998, 54, 3607-3630 and WO 98/39352 and WO 99/14226). The L isomer of this bicyclic sugar modified nucleoside has also been prepared (Frieden et al., Nucleic Acids Research, 2003, 21, 6365-6372). The 4′-CH₂—S-2′ analog has also been prepared (Kumar et al., Bioorg. Med. Chem. Lett., 1998, 8, 2219-2222), and 2′-amino-LNA (Singh et al., J. Org. Chem., 1998, 63, 10035-10039).

Oligomeric compounds of the present invention can also include one or more terminal phosphate moieties. Terminal phosphate moieties can be located at any terminal nucleoside but are suitable at 5′-terminal nucleosides with the 5′-terminal nucleoside of the antisense strand are also suitable. In one aspect, the terminal phosphate is unmodified having the formula —O—P(═O)(OH)OH. In another aspect, the terminal phosphate is modified such that one or more of the O and OH groups are replaced with H, O, S, N(R) or alkyl where R is H, an amino protecting group or unsubstituted or substituted alkyl.

The term “alkyl,” as used herein, refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms with from 1 to about 6 carbon atoms are also suitable. Alkyl groups as used herein may optionally include one or more further substituent groups.

The term “alkenyl,” as used herein, refers to a straight or branched hydrocarbon chain radical containing up to twenty four carbon atoms having at least one carbon-carbon double bond. Examples of alkenyl groups include, but are not limited to, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms are also suitable. Alkenyl groups as used herein may optionally include one or more further substituent groups.

The term “alkynyl,” as used herein, refers to a straight or branched hydrocarbon radical containing up to twenty four carbon atoms and having at least one carbon-carbon triple bond. Examples of alkynyl groups include, but are not limited to, ethynyl, 1-propynyl, 1-butynyl, and the like. Alkynyl groups typically include from 2 to about 24 carbon atoms, more typically from 2 to about 12 carbon atoms with from 2 to about 6 carbon atoms are also suitable. Alkynyl groups as used herein may optionally include one or more further substituent groups.

The term “aliphatic,” as used herein, refers to a straight or branched hydrocarbon radical containing up to twenty four carbon atoms wherein the saturation between any two carbon atoms is a single, double or triple bond. An aliphatic group can contain from 1 to about 24 carbon atoms, more typically from 1 to about 12 carbon atoms with from 1 to about 6 carbon atoms being desired. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines, for example. Aliphatic groups as used herein may optionally include further substituent groups.

The term “alkoxy,” as used herein, refers to a radical formed between an alkyl group and an oxygen atom wherein the oxygen atom is used to attach the alkoxy group to a parent molecule. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

The terms “halo” and “halogen,” as used herein, refer to an atom selected from fluorine, chlorine, bromine and iodine.

The terms “aryl” and “aromatic,” as used herein, refer to a mono- or polycyclic carbocyclic ring system radical having one or more aromatic rings. Examples of aryl groups include, but not limited to, phenyl, naphthyl, tetrahydronaphthyl, indanyl, idenyl and the like. Aryl groups as used herein may optionally include further substituent groups.

The term “heterocyclic,” as used herein, refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain no heteroatoms. A heterocyclic group typically includes at least one atom selected from sulfur, nitrogen or oxygen. Examples of heterocyclic groups include, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substituent groups.

The terms “substituent and substituent group,” as used herein, are meant to include groups that are typically added to other groups or parent compounds to enhance desired properties or give desired effects. Substituent groups can be protected or unprotected and can be added to one available site or to many available sites in a parent compound. Substituent groups may also be further substituted with other substituent groups and may be attached directly or via a linking group such as an alkyl or hydrocarbyl group to the parent compound. Such substituent groups include without limitation, halogen, hydroxyl, alkyl, alkenyl, alkynyl, acyl (—C(O)Rₐ), carboxyl (—C(O)O—Rₐ), aliphatic, alicyclic, alkoxy, substituted oxo (—O—Rₐ), aryl, aralkyl, heterocyclic, heteroaryl, heteroarylalkyl, amino (—NR_{b}R_{c}), imino(═NR_{b}), amido (—C(O)NR_{b}R_{c} or —N(R_{b})C(O)Rₐ), azido (—N₃), nitro (—NO₂), cyano (—CN), carbamido (—OC(O)NR_{b}R_{c} or —N(R_{b})C(O)ORₐ), ureido (—N(R_{b})C(O)NR_{b}R_{c}), thioureido (—N(R_{b})C—(S)NR_{b}R_{c}), guanidinyl (—N(R_{b})C(═NR_{b})NR_{b}R_{c}), amidinyl (—C(═NR_{b})NR_{b}R_{c} or —N(R_{b})C(NR_{b})Rₐ), thiol (—SR_{b}), sulfinyl (—S(O)R_{b}), sulfonyl (—S(O)₂R_{b}) and sulfonamidyl (—S(O)₂NR_{b}R_{c} or —N(R_{b})S(O)₂R_{b}). Wherein each Rₐ, R_{b} and R_{c} is a further substituent group which can be without limitation alkyl, alkenyl, alkynyl, aliphatic, alkoxy, acyl, aryl, aralkyl, heteroaryl, alicyclic, heterocyclic and heteroarylalkyl.

The term “protecting group,” as used herein, refers to a labile chemical moiety which is known in the art to protect reactive groups including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999).

Examples of hydroxylprotecting groups include, but are not limited to, benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl (BOC), isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl (Alloc), acetyl (Ac), formyl, chloroacetyl, trifluoroacetyl, methoxyacetyl, phenoxyacetyl, benzoyl (Bz), methyl, t-butyl, 2,2,2-trichloroethyl, 2-trimethylsilyl ethyl, 1,1-dimethyl-2-propenyl, 3-methyl-3-butenyl, allyl, benzyl (Bn), para-methoxybenzyldiphenylmethylw, triphenylmethyl (trityl), 4,4′-dimethoxy-triphenylmethyl (DMT), substituted or unsubstituted 9-(9-phenyl)xanthenyl (pixyl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, and the like. Suitable hydroxylprotecting groups for the present invention are DMT and substituted or unsubstituted pixyl.

Examples of amino protecting groups include, but are not limited to, t-butoxycarbonyl (BOC), 9-fluorenylmethoxycarbonyl (Fmoc), benzyloxycarbonyl, and the like. Examples of thiol protecting groups include, but are not limited to, triphenylmethyl (Trt), benzyl (Bn), and the like.

The synthesized oligomeric compounds can be separated from a reaction mixture and further purified by a method such as column chromatography, high pressure liquid chromatography, precipitation, or recrystallization. Further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

The term “nucleoside,” as used herein, refers to a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. The term nucleoside is intended to include both modified and unmodified nucleosides. Within the oligonucleotide structure, the phosphate groups are commonly referred to as forming the backbone of the oligomeric compound. In forming oligonucleotides, the phosphate groups covalently link adjacent nucleosides to one another to form a linear polymeric compound. The normal internucleoside linkage of RNA and DNA is a 3′ to 5′ phosphodiester linkage.

In the context of this invention, the term “oligonucleoside” refers to a sequence of nucleosides that are joined by internucleoside linkages that do not have phosphorus atoms. Internucleoside linkages of this type are further described in the “modified internucleoside linkage” section below.

The term “oligonucleotide,” as used herein, refers to an oligomer or polymer of ribonucleic acid (RNA) or deoxyribonucleic acid (DNA) composed of naturally occurring nucleobases, sugars and phosphodiester internucleoside linkages.

The terms “oligomer” and “oligomeric compound,” as used herein, refer to a plurality of naturally occurring and/or non-naturally occurring nucleosides, joined together with internucleoside linking groups in a specific sequence. At least some of the oligomeric compounds can be capable of hybridizing a region of a target nucleic acid. Included in the terms “oligomer” and “oligomeric compound” are oligonucleotides, oligonucleotide analogs, oligonucleotide mimetics, oligonucleosides and chimeric combinations of these. As such the term oligomeric compound is broader than the term “oligonucleotide,” including all oligomers having all manner of modifications including but not limited to those known in the art. Oligomeric compounds are typically structurally distinguishable from, yet functionally interchangeable with, naturally-occurring or synthetic wild-type oligonucleotides. Thus, oligomeric compounds include all such structures that function effectively to mimic the structure and/or function of a desired RNA or DNA strand, for example, by hybridizing to a target. Such non-naturally occurring oligonucleotides are often desired over the naturally occurring forms because they often have enhanced properties, such as for example, enhanced cellular uptake, enhanced affinity for nucleic acid target and increased stability in the presence of nucleases.

Oligomeric compounds can include compositions comprising double-stranded constructs such as, for example, two oligomeric compounds forming a double stranded hybridized construct or a single strand with sufficient self complementarity to allow for hybridization and formation of a fully or partially double-stranded compound. In one embodiment of the invention, double-stranded oligomeric compounds encompass short interfering RNAs (siRNAs). As used herein, the term “siRNA” is defined as a double-stranded construct comprising a first and second strand and having a central complementary portion between the first and second strands and terminal portions that are optionally complementary between the first and second strands or with a target nucleic acid. Each strand in the complex may have a length or from about 12 to about 24 nucleosides and may further comprise a central complementary portion having one of these defined lengths. Each strand may further comprise a terminal unhybridized portion having from 1 to about 6 nucleobases in length. The siRNAs may also have no terminal portions (overhangs) which is referred to as being blunt ended. The two strands of an siRNA can be linked internally leaving free 3′ or 5′ termini or can be linked to form a continuous hairpin structure or loop. The hairpin structure may contain an overhang on either the 5′ or 3′ terminus producing an extension of single-stranded character.

In one embodiment of the invention, compositions comprising double-stranded constructs are canonical siRNAs. As used herein, the term “canonical siRNA” is defined as a double-stranded oligomeric compound having a first strand and a second strand each strand being 21 nucleobases in length with the strands being complementary over 19 nucleobases and having on each 3′ termini of each strand a deoxy thymidine dimer (dTdT) which in the double-stranded compound acts as a 3′ overhang. In another aspect compositions comprise double-stranded constructs having overhangs may be of varying lengths with overhangs of varying lengths and may include compostions wherein only one strand has an overhang.

In another embodiment, compositions comprising double-stranded constructs are blunt-ended siRNAs. As used herein the term “blunt-ended siRNA” is defined as an siRNA having no terminal overhangs. That is, at least one end of the double-stranded constructs is blunt. siRNAs that have one or more overhangs or that are blunt act to elicit dsRNAse enzymes and trigger the recruitment or activation of the RNAi antisense mechanism. In a further embodiment, single-stranded RNAi (ssRNAi) compounds that act via the RNAi antisense mechanism are contemplated.

Further modifications can be made to the double-stranded compounds and may include conjugate groups attached to one or more of the termini, selected nucleobase positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double-stranded. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

The oligomeric compounds in accordance with this invention comprise from about 8 to about 80 nucleobases (i.e. from about 8 to about 80 linked nucleosides/monomeric subunits, or up to 80 linked nucleosides/monomeric subunits). One of ordinary skill in the art will appreciate that the invention embodies oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length, or any range therewithin.

In one embodiment, the oligomeric compounds of the invention are 10 to 50 nucleobases in length, or up to 50 nucleobases in length. One having ordinary skill in the art will appreciate that this embodies oligomeric compounds of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length, or any range therewithin.

In another embodiment, the oligomeric compounds of the invention are 12 to 30 nucleobases in length, or up to 30 nucleobases in length. One having ordinary skill in the art will appreciate that this embodies oligomeric compounds of 12, 13, 14, 15, 16, 17, 18, 19, 20, 21, 22, 23, 24, 25, 26, 27, 28, 29, or 30 nucleobases in length, or any range therewithin.

In another embodiment, the oligomeric compounds of the invention are 17 to 23 nucleobases in length, or up to 23 nucleobases in length. One having ordinary skill in the art will appreciate that this embodies oligomeric compounds of 17, 18, 19, 20, 21, 22 or 23 nucleobases in length, or any range therewithin.

In another embodiment, the oligomeric compounds of the invention are 19 to 21 nucleobases in length, or up to 21 nucleobases in length. One having ordinary skill in the art will appreciate that this embodies oligomeric compounds of 19, 20 or 21 nucleobases in length, or any range therewithin.

As used herein the term “heterocyclic base moiety” refers to nucleobases and modified or substitute nucleobases used to form nucleosides of the invention. The term “heterocyclic base moiety” includes unmodified nucleobases such as the native purine bases adenine (A) and guanine (G), and the pyrimidine bases thymine (T), cytosine (C) and uracil (U). The term is also intended to include all manner of modified or substitute nucleobases including but not limited to synthetic and natural nucleobases such as xanthine, hypoxanthine, 2-aminopyridine and 2-pyridone, 5-methylcytosine (5-me-C), 5-hydroxymethylenyl cytosine, 2-amino and 2-fluoroadenine, 2-propyl and other alkyl derivatives of adenine and guanine, 2-thio cytosine, uracil, thymine, 3-deaza guanine and adenine, 4-thiouracil, 5-uracil (pseudouracil), 5-propynyl (—C≡C—CH₃) uracil and cytosine and other alkynyl derivatives of pyrimidine bases, 5-halo particularly 5-bromo, 5-trifluoromethyl and other 5-substituted uracils and cytosines, 6-methyl and other alkyl derivatives of adenine and guanine, 6-azo uracil, cytosine and thymine, 7-methyl adenine and guanine, 7-deaza adenine and guanine, 8-halo, 8-amino, 8-aza, 8-thio, 8-thioalkyl, 8-hydroxyl and other 8-substituted adenines and guanines, universal bases, hydrophobic bases, promiscuous bases, size-expanded bases, and fluorinated bases as defined herein. Further modified nucleobases include tricyclic pyrimidines such as phenoxazine cytidine(1H-pyrimido[5,4-b][1,4]benzoxazin-2(3H)-one) and phenothiazine cytidine (1H-pyrimido[5,4-b][1,4]benzothiazin-2(3H)-one).

Further nucleobases (and nucleosides comprising the nucleobases) include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, those disclosed in Limbach et al., Nucleic Acids Research, 1994, 22(12), 2183-2196, and those disclosed by Sanghvi, Y. S., Chapter 15, Antisense Research and Applications, pages 289-302, Crooke, S. T. and Lebleu, B., ed., CRC Press, 1993.

Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are especially useful when combined with 2′-O-methoxyethyl (2′-MOE) sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941, and 5,750,692.

The term “universal base” as used herein, refers to a moiety that may be substituted for any base. The universal base need not contribute to hybridization, but should not significantly detract from hybridization and typically refers to a monomer in a first sequence that can pair with a naturally occuring base, i.e A, C, G, T or U at a corresponding position in a second sequence of a duplex in which one or more of the following is true: (1) there is essentially no pairing (hybridization) between the two; or (2) the pairing between them occurs non-discriminant with the universal base hybridizing one or more of the naturally occurring bases and without significant destabilization of the duplex. Exemplary universal bases include, without limitation, inosine, 5-nitroindole and 4-nitrobenzimidazole. For further examples and descriptions of universal bases see Survey and summary: the applications of universal DNA base analogs. Loakes, Nucleic Acids Research, 2001, 29, 12, 2437-2447.

The term “promiscuous base” as used herein, refers to a monomer in a first sequence that can pair with a naturally occuring base, i.e A, C, G, T or U at a corresponding position in a second sequence of a duplex in which the promiscuous base can pair non-discriminantly with more than one of the naturally occurring bases, i.e. A, C, G, T, U. Non-limiting examples of promiscuous bases are 6H,8H-3,4-dihydropyrimido[4,5-c][1,2]oxazin-7-one and N⁶-methoxy-2,6-diaminopurine, shown below. For further information, see Polymerase recognition of synthetic oligodeoxyribonucleotides incorporating degenerate pyriridine and purine bases. Hill, et al., Proc. Natl. Acad. Sci., 1998, 95, 4258-4263.

Examples of G-clamps include substituted phenoxazine cytidine (e.g. 9-(2-aminoethoxy)-H-pyrimido [5,4-b][1,4]benzoxazin-2(3H)-one), carbazole cytidine (2H-pyrirido[4,5-b]indol-2-one) and pyridoindole cytidine (H-pyrido[3′,2′:4,5]pyrrolo[2,3-d]pyrimidin-2-one).

Representative cytosine analogs that make 3 hydrogen bonds with a guanosine in a second oligonucleotide include 1,3-diazaphenoxazine-2-one (Kurchavov et al., Nucleosides and Nucleotides, 1997, 16, 1837-1846), 1,3-diazaphenothiazine-2-one (Lin et al., J. Am. Chem. Soc. 1995, 117, 3873-3874) and 6,7,8,9-tetrafluoro-1,3-diazaphenoxazine-2-one (Wang et al., Tetrahedron Lett. 1998, 39, 8385-8388). When incorporated into oligonucleotides these base modifications hybridized with complementary guanine (the latter also hybridized with adenine) and enhanced helical thermal stability by extended stacking interactions (see U.S. Ser. No. 10/013,295).

Oligomeric compounds of the invention may also contain one or more substituted sugar moieties such as the 2′-modified sugars discussed. A more comprehensive but not limiting list of sugar substituent groups includes: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Some oligonucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. One modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other sugar substituent groups include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-Sugar substituent groups may be in the arabino (up) position or ribo (down) position. One 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920.

Representative sugar substituent groups include groups of formula Iₐ or IIₐ: 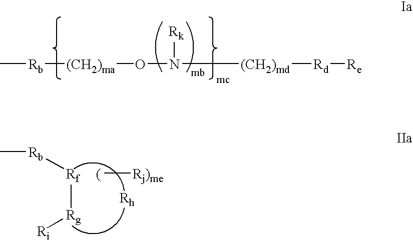
wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ;

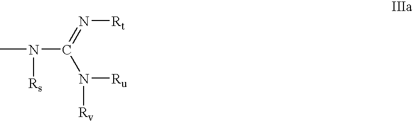

Rₚ and R_{q} are each independently hydrogen or C₁-C₁₀ alkyl;

Rᵣ is —Rₓ-R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, a nitrogen protecting group or —Rₓ-R_{y};

Rₚ is hydrogen, a nitrogen protecting group or —Rₓ-R_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where the acyl is an acid amide or an ester;

or Rₘ and Rₙ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ, or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein the heteroatoms are selected from oxygen, nitrogen and sulfur and wherein the ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;

mₐ is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Representative substituents groups of Formula I are disclosed in U.S. Ser. No. 09/130,973, filed Aug. 7, 1998, entitled “Capped 2′-Oxyethoxy Oligonucleotides.”

Representative cyclic substituent groups of Formula II are disclosed in U.S. Ser. No. 09/123,108, filed Jul. 27, 1998, entitled “RNA Targeted 2′-Oligomeric compounds that are Conformationally Preorganized”.

Particular sugar substituent groups include O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃))₂, where n and m are from 1 to about 10.

Representative guanidino substituent groups that are shown in formula III and IV are disclosed in U.S. Ser. No. 09/349,040, entitled “Functionalized Oligomers”, filed Jul. 7, 1999.

Representative acetamido substituent groups are disclosed in U.S. Pat. No. 6,147,200.

Representative dimethylaminoethyloxyethyl substituent groups are disclosed in International Patent Application PCT/US99/17895, entitled “2′-O-Dimethylaminoethyloxyethyl-Oligomeric compounds”, filed Aug. 6, 1999.

The terms “modified internucleoside linkage” and “modified backbone,” or simply “modified linkage” as used herein, refer to modifications or replacement of the naturally occurring phosphodiester internucleoside linkage connecting two adjacent nucleosides within an oligomeric compound. Such modified linkages include those that have a phosphorus atom and those that do not have a phosphorus atom.

Internucleoside linkages containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity can comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

In the C. elegans system, modification of the internucleotide linkage (phosphorothioate in place of phosphodiester) did not significantly interfere with RNAi activity, indicating that oligomeric compounds of the invention can have one or more modified internucleoside linkages, and retain activity. Indeed, such modified internucleoside linkages are often desired over the naturally occurring phosphodiester linkage because of advantageous properties they can impart such as, for example, enhanced cellular uptake, enhanced affinity for nucleic acid target and increased stability in the presence of nucleases.

Another phosphorus containing modified internucleoside linkage is the phosphonomonoester (see U.S. Pat. Nos. 5,874,553 and 6,127,346). Phosphonomonoester nucleic acids have useful physical, biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

As previously defined an oligonucleoside refers to a sequence of nucleosides that are joined by internucleoside linkages that do not have phosphorus atoms. Non-phosphorus containing internucleoside linkages include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

Some additional examples of modified internucleoside linkages that do not contain a phosphorus atom therein include, —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type and amide internucleoside linkages are disclosed in the below referenced U.S. Pat. Nos. 5,489,677 and 5,602,240, respectively.

Another modification that can enhance the properties of an oligomeric compound or can be used to track the oligomeric compound or its metabolites is the attachment of one or more moieties or conjugates. Properties that are typically enhanced include without limitation activity, cellular distribution and cellular uptake. In one embodiment, such modified oligomeric compounds are prepared by covalently attaching conjugate groups to functional groups available on an oligomeric compound such as hydroxyl or amino functional groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve properties including but not limited to oligomer uptake, enhance oligomer resistance to degradation, and/or strengthen sequence-specific hybridization with RNA. Groups that enhance the pharmacokinetic properties, in the context of this invention, include groups that improve properties including but not limited to oligomer uptake, distribution, metabolism and excretion. Representative conjugate groups are disclosed in International Patent Application PCT/US92/09196.

Conjugate groups include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

The oligomeric compounds of the invention may also be conjugated to active drug substances, for example, aspirin, warfarin, phenylbutazone, ibuprofen, suprofen, fenbufen, ketoprofen, (S′)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130.

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Oligomeric compounds used in the compositions of the present invention can also be modified to have one or more stabilizing groups that are generally attached to one or both terimini of oligomeric compounds to enhance properties such as for example nuclease stability. Included in stabilizing groups are cap structures. The terms “cap structure” or “terminal cap moiety,” as used herein, refer to chemical modifications, which can be attached to one or both of the termini of an oligomeric compound. These terminal modifications protect the oligomeric compounds having terminal nucleic acid moieties from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270).

Particularly suitable 3′-cap structures of the present invention include, for example 4′,5′-methylene nucleotide; 1-(beta-D-erythrofaranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925 and Published U.S. Patent Application Publication No. US 2005/0020525 published on Jan. 27, 2005). Further 3′ and 5′-stabilizing groups that can be used to cap one or both ends of an oligomeric compound to impart nuclease stability include those disclosed in WO 03/004602.

Oligomerization of modified and unmodified nucleosides is performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. In addition specific protocols for the synthesis of oligomeric compounds of the invention are illustrated in the examples below.

Support bound oligonucleotide synthesis relies on sequential addition of nucleotides to one end of a growing chain. Typically, a first nucleoside (having protecting groups on any exocyclic amine functionalities present) is attached to an appropriate glass bead support and nucleotides bearing the appropriate activated phosphite moiety, i.e. an “activated phosphorous group” (typically nucleotide phosphoramidites, also bearing appropriate protecting groups) are added stepwise to elongate the growing oligonucleotide. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069.

Oligonucleotides are generally prepared either in solution or on a support medium, e.g. a solid support medium. In general a first synthon (e.g. a monomer, such as a nucleoside) is first attached to a support medium, and the oligonucleotide is then synthesized by sequentially coupling monomers to the support-bound synthon. This iterative elongation eventually results in a final oligomeric compound or other polymer such as a polypeptide. Suitable support medium can be soluble or insoluble, or may possess variable solubility in different solvents to allow the growing support bound polymer to be either in or out of solution as desired. Traditional support medium such as solid support media are for the most part insoluble and are routinely placed in reaction vessels while reagents and solvents react with and/or wash the growing chain until the oligomer has reached the target length, after which it is cleaved from the support and, if necessary further worked up to produce the final polymeric compound. More recent approaches have introduced soluble supports including soluble polymer supports to allow precipitating and dissolving the iteratively synthesized product at desired points in the synthesis (Gravert et al., Chem. Rev., 1997, 97, 489-510).

The term support medium is intended to include all forms of support known to one of ordinary skill in the art for the synthesis of oligomeric compounds and related compounds such as peptides. Some representative support medium that are amenable to the methods of the present invention include but are not limited to the following: controlled pore glass (CPG); oxalyl-controlled pore glass (see, e.g., Alul, et al., Nucleic Acids Research 1991, 19, 1527); silica-containing particles, such as porous glass beads and silica gel such as that formed by the reaction of trichloro-[3-(4-chloromethyl)phenyl]propylsilane and porous glass beads (see Parr and Grohmann, Angew. Chem. Internal. Ed. 1972, 11, 314, sold under the trademark “PORASIL E” by Waters Associates, Framingham, Mass., USA); the mono ester of 1,4-dihydroxymethylenlybenzene and silica (see Bayer and Jung, Tetrahedron Lett., 1970, 4503, sold under the trademark “BIOPAK” by Waters Associates); TENTAGEL (see, e.g., Wright, et al., Tetrahedron Letters 1993, 34, 3373); cross-linked styrene/divinylbenzene copolymer beaded matrix or POROS, a copolymer of polystyrene/divinylbenzene (available from Perceptive Biosystems); soluble support medium, polyethylene glycol PEGs (see Bonora et al., Organic Process Research & Development, 2000, 4, 225-231).

The term “linking moiety,” as used herein is generally a bi-functional group, covalently binds the ultimate 3′-nucleoside (and thus the nascent oligonucleotide) to the solid support medium during synthesis, but which is cleaved under conditions orthogonal to the conditions under which the 5′-protecting group, and if applicable any 2′-protecting group, are removed. Suitable linking moietys include, but are not limited to, a divalent group such as alkylene, cycloalkylene, arylene, heterocyclyl, heteroarylene, and the other variables are as described above.

Exemplary alkylene linking moietys include, but are not limited to, C₁-C₁₂ alkylene (e.g. methylene, ethylene (e.g. ethyl-1,2-ene), propylene (e.g. propyl-1,2-ene, propyl-1,3-ene), butylene, (e.g. butyl-1,4-ene, 2-methylpropyl-1,3-ene), pentylene, hexylene, heptylene, octylene, decylene, dodecylene), etc. Exemplary cycloalkylene groups include C₃-C₁₂ cycloalkylene groups, such as cyclopropylene, cyclobutylene, cyclopentanyl-1,3-ene, cyclohexyl-1,4-ene, etc. Exemplary arylene linking moietys include, but are not limited to, mono- or bicyclic arylene groups having from 6 to about 14 carbon atoms, e.g. phenyl-1,2-ene, naphthyl-1,6-ene, napthyl-2,7-ene, anthracenyl, etc. Exemplary heterocyclyl groups within the scope of the invention include mono- or bicyclic aryl groups having from about 4 to about 12 carbon atoms and about 1 to about 4 hetero atoms, such as N, O and S, where the cyclic moieties may be partially dehydrogenated.

Certain heteroaryl groups that may be mentioned as being within the scope of the invention include: pyrrolidinyl, piperidinyl (e.g. 2,5-piperidinyl, 3,5-piperidinyl), piperazinyl, tetrahydrothiophenyl, tetrahydrofuranyl, tetrahydro quinolinyl, tetrahydro isoquinolinyl, tetrahydroquinazolinyl, tetrahydroquinoxalinyl, etc. Exemplary heteroarylene groups include mono- or bicyclic aryl groups having from about 4 to about 12 carbon atoms and about 1 to about 4 hetero atoms, such as N, O and S. Certain heteroaryl groups that may be mentioned as being within the scope of the invention include: pyridylene (e.g. pyridyl-2,5-ene, pyridyl-3,5-ene), pyrimidinyl, thiophenyl, furanyl, quinolinyl, isoquinolinyl, quinazolinyl, quinoxalinyl, etc.

Commercially available equipment routinely used for the support medium based synthesis of oligomeric compounds and related compounds is sold by several vendors including, for example, Applied Biosystems (Foster City, Calif.). Any other means for such synthesis known in the art may additionally or alternatively be employed. Suitable solid phase techniques, including automated synthesis techniques, are described in F. Eckstein (ed.), Oligonucleotides and Analogues, a Practical Approach, Oxford University Press, New York (1991).

Although a lot of research has focused on the synthesis of oligoribonucleotides the main RNA synthesis strategies that are presently being used commercially include 5′-O-DMT-2′-O-t-butyldimethylsilyl (TBDMS), 5′-O-DMT-2′-O-[1(2-fluorophenyl)-4-methoxypiperidin-4-yl] (FPMP), 2′-O-[(triisopropylsilyl)oxy]methyl (2′-O—CH₂—O—Si(iPr)₃ (TOM), and the 5′-O-silyl ether-2′-ACE (5′-O-bis(trimethylsiloxy)cyclododecyloxysilyl ether (DOD)-2′-O-bis(2-acetoxyethoxy)methyl (ACE). A current list of some of the major companies currently offering RNA products include Pierce Nucleic Acid Technologies, Dharmacon Research Inc., Ameri Biotechnologies Inc., and Integrated DNA Technologies, Inc. One company, Princeton Separations, is marketing an RNA synthesis activator advertised to reduce coupling times especially with TOM and TBDMS chemistries. Such an activator would also be amenable to the present invention. The primary groups being used for commercial RNA synthesis are:
TBDMS=5′-O-DMT-2′-O-t-butyldimethylsilyl;
TOM=2′-O-[(triisopropylsilyl)oxy]methyl;
DOD/ACE=5′-O-bis(trimethylsiloxy)cyclododecyloxysilylether-2′-O-bis(2-acetoxyethoxy)methyl;
FPMP=5′-O-DMT-2′-O-[1 (2-fluorophenyl)-4-methoxypiperidin-4-yl].

All of the aforementioned RNA synthesis strategies are amenable to the present invention. Strategies that would be a hybrid of the above e.g. using a 5′-protecting group from one strategy with a 2′-O-protecting from another strategy is also amenable to the present invention.

The terms “antisense” or “antisense inhibition” as used herein refer to the hybridization of an oligomeric compound or a portion thereof with a selected target nucleic acid. Multiple antisense mechanisms exist by which oligomeric compounds can be used to modulate gene expression in mammalian cells. Such antisense inhibition is typically based upon hydrogen bonding-based hybridization of complementary strands or segments such that at least one strand or segment is cleaved, degraded, or otherwise rendered inoperable. In this regard, it is presently suitable to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA.

A commonly exploited antisense mechanism is RNase H-dependent degradation of a targeted RNA. RNase H is a ubiquitously expressed endonuclease that recognizes antisense DNA-RNA heteroduplexes, hydrolyzing the RNA strand. A further antisense mechanism involves the utilization of enzymes that catalyze the cleavage of RNA-RNA duplexes. These reactions are catalyzed by a class of RNAse enzymes including but not limited to RNAse III and RNAse L. The antisense mechanism known as RNA interference (RNAi) is operative on RNA-RNA hybrids and the like. Both RNase H-based antisense (usually using single-stranded compounds) and RNA interference (usually using double-stranded compounds known as siRNAs) are antisense mechanisms, typically resulting in loss of target RNA function.

Optimized siRNA and RNase H-dependent oligomeric compounds behave similarly in terms of potency, maximal effects, specificity and duration of action, and efficiency. Moreover it has been shown that in general, activity of dsRNA constructs correlated with the activity of RNase H-dependent single-stranded antisense oligomeric compounds targeted to the same site. One major exception is that RNase H-dependent antisense oligomeric compounds were generally active against target sites in pre-mRNA whereas siRNAs were not.

These data suggest that, in general, sites on the target RNA that were not active with RNase H-dependent oligonucleotides were similarly not good sites for siRNA. Conversely, a significant degree of correlation between active RNase H oligomeric compounds and siRNA was found, suggesting that if a site is available for hybridization to an RNase H oligomeric compound, then it is also available for hybridization and cleavage by the siRNA complex. Consequetly, once suitable target sites have been determined by either antisense approach, these sites can be used to design constructs that operate by the alternative antisense mechanism (Vickers et al., J. Biol. Chem., 2003, 278, 7108). Moreover, once a site has been demonstrated as active for either an RNAi or an RNAse H oligomeric compound, a single-stranded RNAi oligomeric compound (ssRNAi or asRNA) can be designed.

The oligomeric compounds and methods of the present invention are also useful in the study, characterization, validation and modulation of small non-coding RNAs. These include, but are not limited to, microRNAs (miRNA), small nuclear RNAs (snRNA), small nucleolar RNAs (snoRNA), small temporal RNAs (stRNA) and tiny non-coding RNAs (tncRNA) or their precursors or processed transcripts or their association with other cellular components.

Small non-coding RNAs have been shown to function in various developmental and regulatory pathways in a wide range of organisms, including plants, nematodes and mammals. MicroRNAs are small non-coding RNAs that are processed from larger precursors by enzymatic cleavage and inhibit translation of mRNAs. stRNAs, while processed from precursors much like miRNAs, have been shown to be involved in developmental timing regulation. Other non-coding small RNAs are involved in events as diverse as cellular splicing of transcripts, translation, transport, and chromosome organization.

As modulators of small non-coding RNA function, the oligomeric compounds of the present invention find utility in the control and manipulation of cellular functions or processes such as regulation of splicing, chromosome packaging or methylation, control of developmental timing events, increase or decrease of target RNA expression levels depending on the timing of delivery into the specific biological pathway and translational or transcriptional control. In addition, the oligomeric compounds of the present invention can be modified in order to optimize their effects in certain cellular compartments, such as the cytoplasm, nucleus, nucleolus or mitochondria.

The compounds of the present invention can further be used to identify components of regulatory pathways of RNA processing or metabolism as well as in screening assays or devices.

Targeting an oligomeric compound to a particular nucleic acid molecule, in the context of this invention, can be a multistep process. The process usually begins with the identification of a target nucleic acid whose function is to be modulated. The terms “target nucleic acid” and “nucleic acid target”, as used herein, refer to any nucleic acid capable of being targeted including without limitation DNA (a cellular gene), RNA (including pre-mRNA and mRNA or portions thereof) transcribed from such DNA, and also cDNA derived from such RNA. In one embodiment the modulation of expression of a selected gene is associated with a particular disorder or disease state. In another embodiment the target nucleic acid is a nucleic acid molecule from an infectious agent.

The targeting process usually also includes determination of at least one target region, segment, or site within the target nucleic acid for the antisense interaction to occur such that the desired effect, e.g., modulation of expression, will result. Within the context of the present invention as it is applied to a nucleic acid target, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid. The terms region, segment, and site can also be used to describe an oligomeric compound of the invention such as for example a gapped oligomeric compound having 3 separate regions or segments.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding a nucleic acid target, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense oligomeric compounds of the present invention.

The open reading frame (ORF) or “coding region,” which is known in the art to refer to the region between the translation initiation codon and the translation termination codon, is also a region which may be targeted effectively. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also suitable to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also suitable target sites. mRNA transcripts produced via the process of splicing of two (or more) mRNAs from different gene sources are known as “fusion transcripts”. It is also known that introns can be effectively targeted using antisense oligomeric compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequences.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also suitable target nucleic acids.

The locations on the target nucleic acid to which the antisense oligomeric compounds hybridize are hereinbelow referred to as “suitable target segments.” As used herein the term “suitable target segment” is defined as at least an 8-nucleobase portion of a target region to which an active antisense oligomeric compound is targeted. While not wishing to be bound by theory, it is presently believed that these target segments represent portions of the target nucleic acid which are accessible for hybridization.

Exemplary antisense oligomeric compounds include oligomeric compounds that comprise at least the 8 consecutive nucleobases from the 5′-terminus of a targeted nucleic acid e.g. a cellular gene or mRNA transcribed from the gene (the remaining nucleobases being a consecutive stretch of the same oligonucleotide beginning immediately upstream of the 5′-terminus of the antisense oligomeric compound which is specifically hybridizable to the target nucleic acid and continuing until the oligonucleotide contains from about 8 to about 80 nucleobases). Similarly, antisense oligomeric compounds are represented by oligonucleotide sequences that comprise at least the 8 consecutive nucleobases from the 3′-terminus of one of the illustrative antisense oligomeric compounds (the remaining nucleobases being a consecutive stretch of the same oligonucleotide beginning immediately downstream of the 3′-terminus of the antisense oligomeric compound which is specifically hybridizable to the target nucleic acid and continuing until the oligonucleotide contains from about 8 to about 80 nucleobases). One having skill in the art armed with the antisense oligomeric compounds illustrated herein will be able, without undue experimentation, to identify further antisense oligomeric compounds.

Once one or more target regions, segments or sites have been identified, antisense oligomeric compounds are chosen which are sufficiently complementary to the target, i.e., hybridize sufficiently well and with sufficient specificity, to give the desired effect.

In accordance with one embodiment of the present invention, a series of nucleic acid duplexes comprising the antisense oligomeric compounds of the present invention and their complements can be designed for a specific target or targets. The ends of the strands may be modified by the addition of one or more natural or modified nucleobases to form an overhang. The sense strand of the duplex is then designed and synthesized as the complement of the antisense strand and may also contain modifications or additions to either terminus. For example, in one embodiment, both strands of the duplex would be complementary over the central nucleobases, each having overhangs at one or both termini.

RNA strands of the duplex can be synthesized by methods disclosed herein or purchased from various RNA synthesis companies such as for example Dharmacon Research Inc., (Lafayette, Colo.). Once synthesized, the complementary strands are annealed. The single strands are aliquoted and diluted to a concentration of 50 μM. Once diluted, 30 μL of each strand is combined with 15 μL of a 5× solution of annealing buffer. The final concentration of the buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA compound is 20 μM. This solution can be stored frozen (−20° C.) and freeze-thawed up to 5 times.

Once prepared, the desired synthetic duplexs are evaluated for their ability to modulate target expression. When cells reach 80% confluency, they are treated with synthetic duplexs comprising at least one oligomeric compound of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired dsRNA compound at a final concentration of 200 nM. After 5 hours of treatment, the medium is replaced with fresh medium. Cells are harvested 16 hours after treatment, at which time RNA is isolated and target reduction measured by RT-PCR.

In a further embodiment, the “suitable target segments” identified herein may be employed in a screen for additional oligomeric compounds that modulate the expression of a target. “Modulators” are those oligomeric compounds that decrease or increase the expression of a nucleic acid molecule encoding a target and which comprise at least an 8-nucleobase portion which is complementary to a suitable target segment. The screening method comprises the steps of contacting a suitable target segment of a nucleic acid molecule encoding a target with one or more candidate modulators, and selecting for one or more candidate modulators which decrease or increase the expression of a nucleic acid molecule encoding a target. Once it is shown that the candidate modulator or modulators are capable of modulating (e.g. either decreasing or increasing) the expression of a nucleic acid molecule encoding a target, the modulator may then be employed in further investigative studies of the function of a target, or for use as a research, diagnostic, or therapeutic agent in accordance with the present invention.

The suitable target segments of the present invention may also be combined with their respective complementary antisense oligomeric compounds of the present invention to form stabilized double stranded (duplexed) oligonucleotides.

In the context of this invention, “hybridization” means hydrogen bonding, which may be Watson-Crick, Hoogsteen or reversed Hoogsteen hydrogen bonding, between the heterocyclic base moieties of complementary nucleosides. For example, adenine and thyrnine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense oligomeric compound need not be 100% complementary to that of its target nucleic acid to be specifically hybridizable. An antisense oligomeric compound is specifically hybridizable when binding of the compound to the target DNA or RNA molecule interferes with the normal function of the target DNA or RNA to cause a complete or partial loss of function, and there is a sufficient degree of complementarity to avoid non-specific binding of the antisense oligomeric compound to non-target sequences under conditions in which specific binding is desired, i.e., under physiological conditions in the case of therapeutic treatment, or under conditions in which in vitro or in vivo assays are performed. Moreover, an oligonucleotide may hybridize over one or more segments such that intervening or adjacent segments are not involved in the hybridization event (e.g., a loop structure, mismatch or hairpin structure).

The oligomeric compounds of the present invention comprise at least 70%, at least 75%, at least 80%, at least 85%, at least 90%, at least 95%, at least 99%, or 100% sequence complementarity to a target region within the target nucleic acid sequence to which they are targeted. For example, an antisense oligomeric compound in which 18 of 20 nucleobases of the antisense oligomeric compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention.

Percent complementarity of an antisense oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, homology, sequence identity or complementarity, between the oligomeric compound and the target is about 70%, about 75%, about 80%, about 85%, about 90%, about 92%, about 94%, about 95%, about 96%, about 97%, about 98%, about 99%, or 100%.

In some embodiments, “suitable target segments” may be employed in a screen for additional oligomeric compounds that modulate the expression of a selected protein. “Modulators” are those oligomeric compounds that decrease or increase the expression of a nucleic acid molecule encoding a protein and which comprise at least an 8-nucleobase portion which is complementary to a suitable target segment. The screening method comprises the steps of contacting a suitable target segment of a nucleic acid molecule encoding a protein with one or more candidate modulators, and selecting for one or more candidate modulators which decrease or increase the expression of a nucleic acid molecule encoding a protein. Once it is shown that the candidate modulator or modulators are capable of modulating (e.g. either decreasing or increasing) the expression of a nucleic acid molecule encoding a peptide, the modulator may then be employed in further investigative studies of the function of the peptide, or for use as a research, diagnostic, or therapeutic agent in accordance with the present invention.

The suitable target segments of the present invention may also be combined with their respective complementary antisense oligomeric compounds of the present invention to form stabilized double stranded (duplexed) oligonucleotides. Such double stranded oligonucleotide moieties have been shown in the art to modulate target expression and regulate translation as well as RNA processsing via an antisense mechanism. Moreover, the double stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double stranded moieties have been shown to inhibit the target by the classical hybridization of antisense strand of the duplex to the target, thereby triggering enzymatic degradation of the target (Tijsterman et al., Science, 2002, 295, 694-697). The oligomeric compounds of the present invention can also be applied in the areas of drug discovery and target validation. The present invention comprehends the use of the oligomeric compounds and targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

Effect of nucleoside modifications on RNAi activity can be evaluated according to existing literature (Elbashir et al., Nature, 2001, 411, 494-498; Nishikura et al., Cell, 2001, 107, 415-416; and Bass et al., Cell, 2000, 101, 235-238.)

The oligomeric compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway. For use in kits and diagnostics, the oligomeric compounds of the present invention, either alone or in combination with other oligomeric compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense oligomeric compounds are compared to control cells or tissues not treated with antisense oligomeric compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds and or oligomeric compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The oligomeric compounds of the invention are useful for research and diagnostics, in one aspect because they hybridize to nucleic acids encoding proteins. For example, oligonucleotides that are shown to hybridize with such efficiency and under such conditions as disclosed herein as to be effective protein inhibitors will also be effective primers or probes under conditions favoring gene amplification or detection, respectively. These primers and probes are useful in methods requiring the specific detection of nucleic acid molecules encoding proteins and in the amplification of the nucleic acid molecules for detection or for use in further studies. Hybridization of the antisense oligonucleotides, particularly the primers and probes, of the invention with a nucleic acid can be detected by means known in the art. Such means may include conjugation of an enzyme to the oligonucleotide, radiolabelling of the oligonucleotide or any other suitable detection means. Kits using such detection means for detecting the level of selected proteins in a sample may also be prepared.

The specificity and sensitivity of antisense is also harnessed by those of skill in the art for therapeutic uses. Antisense oligomeric compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense oligomeric compounds can be useful therapeutic modalities that can be configured to be useful in treatment regimes for the treatment of cells, tissues and animals, especially humans.

As used herein, the term “patient” refers to a mammal that is afflicted with one or more disorders associated with expression or overexpression of one or more genes. It will be understood that the most suitable patient is a human. It is also understood that this invention relates specifically to the inhibition of mammalian expression or overexpression of one or more genes.

It is recognized that one skilled in the art may affect the disorders associated with expression or overexpression of a gene by treating a patient presently afflicted with the disorders with an effective amount of one or more oligomeric compounds or compositions of the present invention. Thus, the terms “treatment” and “treating” are intended to refer to all processes wherein there may be a slowing, interrupting, arresting, controlling, or stopping of the progression of the disorders described herein, but does not necessarily indicate a total elimination of all symptoms.

As used herein, the term “effective amount” or “therapeutically effective amount” of a compound of the present invention refers to an amount that is effective in treating or preventing the disorders described herein.

For therapeutics, a patient, such as a human, suspected of having a disease or disorder which can be treated by modulating the expression of a gene is treated by administering antisense oligomeric compounds in accordance with this invention. The compounds of the invention can be utilized in pharmaceutical compositions by adding an effective amount of an antisense oligomeric compound to a suitable pharmaceutically acceptable diluent or carrier. Use of the antisense oligomeric compounds and methods of the invention may also be useful prophylactically, e.g., to prevent or delay infection, inflammation or tumor formation, for example. In some embodiments, the patient being treated has been identified as being in need of treatment or has been previously diagnosed as such.

The oligomeric compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. For oligonucleotides, examples of pharmaceutically acceptable salts and their uses are further described in U.S. Pat. No. 6,287,860.

The compositions of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative U.S. patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The present invention also includes pharmaceutical compositions and formulations which include the compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

One of skill in the art will recognize that formulations are routinely designed according to their intended use, i.e. route of administration.

Suitable formulations for topical administration include those in which the oligonucleotides of the invention are in admixture with a topical delivery agent such as lipids, liposomes, fatty acids, fatty acid esters, steroids, chelating agents and surfactants. Suitable lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Penetration enhancers and their uses are further described in U.S. Pat. No. 6,287,860. Surfactants and their uses are further described in U.S. Pat. No. 6,287,860.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Suitable oral formulations are those in which oligonucleotides of the invention are administered in conjunction with one or more penetration enhancers surfactants and chelators. Suitable surfactants include fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. A particularly suitable combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents and their uses are further described in U.S. Pat. No. 6,287,860. Oral formulations for oligonucleotides and their preparation are described in detail in U.S. application Ser. Nos. 09/108,673 (filed Jul. 1, 1998), 09/315,298 (filed May 20, 1999) and 10/071,822, filed Feb. 8, 2002.

In another related embodiment, therapeutically effective combination therapies may comprise the use of two or more compositions of the invention wherein the multiple compositions are targeted to a single or multiple nucleic acid targets. Numerous examples of antisense oligomeric compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, and may be given once or more daily, weekly, monthly or yearly. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, weekly, monthly, or yearly. For double-stranded compounds, the dose must be calculated to account for the increased nucleic acid load of the second strand (as with compounds comprising two separate strands) or the additional nucleic acid length (as with self complementary single strands having double-stranded regions).

While the present invention has been described with specificity in accordance with certain of its embodiments, the following examples serve only to illustrate the invention and are not intended to limit the same.

### EXAMPLES

General

The sequences listed in the examples have been annotated to indicate where there are modified nucleosides or internucleoside linkages. All non-annotated nucleosides are β-D-ribonucleosides linked by phosphodiester internucleoside linkages. Phosphorothioate internucleoside linkages are indicated by underlining. Modified nucleosides are indicated by a subscripted letter following the capital letter indicating the nucleoside. In particular, subscript “f” indicates 2′-fluoro; subscript “m” indicates 2′-O-methyl; subscript “1” indicates LNA; subscript “e” indicates 2′-O-methoxyethyl (MOE); and subscript “t” indicates 4′-thio. For example Uₘ is a modified uridine having a 2′-OCH₃ group. A “d” preceding a nucleoside indicates a deoxynucleoside such as dT which is deoxythymidine. Some of the strands have a 5′-phosphate group designated as “P—”. Bolded and italicized “C” indicates a 5-methyl C ribonucleoside. Where noted next to the ISIS number of a compound, “as” designates the antisense strand, and “s” designates the sense strand of the duplex, with respect to the target sequence.

### Example 1

### Synthesis of Nucleoside Phosphoramidites

The preparation of nucleoside phosphoramidites is performed following procedures that are 30 extensively illustrated in the art such as but not limited to U.S. Pat. No. 6,426,220 and published PCT WO 02/36743.

### Example 2

### Oligonucleotide and Oligonucleoside Synthesis

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NILOAc solution. Phosphinate oligonucleotides are prepared as described in U.S. Pat. No. 5,508,270.

Alkyl phosphonate oligonucleotides are prepared as described in U.S. Pat. No. 4,469,863.

3′-Deoxy-3′-methylene phosphonate oligonucleotides are prepared as described in U.S. Pat. No. 5,610,289 or 5,625,050.

Phosphoramidite oligonucleotides are prepared as described in U.S. Patent, 5,256,775 or U.S. Pat. No. 5,366,878.

Alkylphosphonothioate oligonucleotides are prepared as described in published PCT applications PCT/US94/00902 and PCT/US93/06976 (published as WO 94/17093 and WO 94/02499, respectively).

3′-Deoxy-3′-amino phosphoramidate oligonucleotides are prepared as described in U.S. Pat. No. 5,476,925.

Phosphotriester oligonucleotides are prepared as described in U.S. Pat. No. 5,023,243.

Borano phosphate oligonucleotides are prepared as described in U.S. Pat. Nos. 5,130,302 and 5,177,198.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages are prepared as described in U.S. Pat. Nos. 5,378,825, 5,386,023, 5,489,677, 5,602,240 and 5,610,289.

Formacetal and thioformacetal linked oligonucleosides are prepared as described in U.S. Pat. Nos. 5,264,562 and 5,264,564.

Ethylene oxide linked oligonucleosides are prepared as described in U.S. Pat. No. 5,223,618.

### Example 3

### Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 4

### Oligonucleotide Synthesis—96 Well Plate Format

Oligonucleotides can be synthesized via solid phase P(III) phosphoramidite chemistry on an automated synthesizer capable of assembling 96 sequences simultaneously in a 96-well format. Phosphodiester internucleotide linkages are afforded by oxidation with aqueous iodine. Phosphorothioate internucleotide linkages are generated by sulfurrization utilizing 3,H-1,2 benzodithiole-3-one 1,1 dioxide (Beaucage Reagent) in anhydrous acetonitrile. Standard base-protected beta-cyanoethyl-diiso-propyl phosphorarnidites are purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides are cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Example 5

### Oligonucleotide Analysis Using 96-Well Plate Format

The concentration of oligonucleotide in each well is assessed by dilution of samples and UV absorption spectroscopy. The full-length integrity of the individual products is evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition is confirmed by mass analysis of the oligomeric compounds utilizing electrospray-mass spectroscopy. All assay test plates are diluted from the master plate using single and multi-channel robotic pipettors. Plates are judged to be acceptable if at least 85% of the oligomeric compounds on the plate are at least 85% full length.

### Example 6

### Cell Culture and Oligonucleotide Treatment

The effect of oligomeric compounds on target nucleic acid expression can be tested in any of a variety of cell types provided that the target nucleic acid is present at measurable levels. This can be routinely determined using, for example, PCR or Northern blot analysis. Cell lines derived from multiple tissues and species can be obtained from American Type Culture Collection (ATCC, Manassas, Va.).

The following cell types are provided for illustrative purposes, but other cell types can be routinely used, provided that the target is expressed in the cell type chosen. This can be readily determined by methods routine in the art, for example Northern blot analysis, ribonuclease protection assays or RT-PCR.

T-24 cells: The human transitional cell bladder carcinoma cell line T-24 is obtained from the American Type Culture Collection (ATCC) (Manassas, Va.). T-24 cells are routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells are routinely passaged by trypsinization and dilution when they reached 90% confluence. Cells are seeded into 96-well plates (Falcon-Primaria #353872) at a density of 7000 cells/well for uses including but not limited to oligomeric compound transfection experiments.

A549 cells: The human lung carcinoma cell line A549 was obtained from the American Type Culture Collection (Manassas, Va.). A549 cells were routinely cultured in DMEM, high glucose (Invitrogen Life Technologies, Carlsbad, Calif.) supplemented with 10% fetal bovine serum, 100 units per ml penicillin, and 100 micrograms per ml streptomycin (Invitrogen Life Technologies, Carlsbad, Calif.). Cells were routinely passaged by trypsinization and dilution when they reached approximately 90% confluence. Cells were seeded into 96-well plates (Falcon-Primaria #3872) at a density of approximately 5000 cells/well for uses including but not limited to oligomeric compound transfection experiments.

b.END cells: The mouse brain endothelial cell line b.END was obtained from Dr. Werner Risau at the Max Plank Institute (Bad Nauheim, Germany). b.END cells were routinely cultured in DMEM, high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 3000 cells/well for uses including but not limited to oligomeric compound transfection experiments.

HeLa cells: The human epitheloid carcinoma cell line HeLa was obtained from the American Tissue Type Culture Collection (Manassas, Va.). HeLa cells were routinely cultured in DMEM, high glucose (Invitrogen Corporation, Carlsbad, Calif.) supplemented with 10% fetal bovine serum (Invitrogen Corporation, Carlsbad, Calif.). Cells were routinely passaged by trypsinization and dilution when they reached 90% confluence. Cells were seeded into 24-well plates (Falcon-Primaria #3846) at a density of 50,000 cells/well or in 96-well plates at a density of 5,000 cells/well for uses including but not limited to oligomeric compound transfection experiments.

MH-S cells: The mouse alveolar macrophage cell line was obtained from American Type Culture Collection (Manassas, Va.). MH-S cells were cultured in RPMI Medium 1640 with L-glutamine(Invitrogen Life Technologies, Carlsbad, Calif.), supplemented with 10% fetal bovine serum, 1 mM sodium pyruvate and 10 mM HEPES (all supplements from Invitrogen Life Technologies, Carlsbad, Calif.). Cells were routinely passaged by trypsinization and dilution when they reached 70-80% confluence. Cells were seeded into 96-well plates (Falcon-Primaria #353047, BD Biosciences, Bedford, Mass.) at a density of 6500 cells/well for uses including but not limited to oligomeric compound transfection experiments.

U-87 MG: The human glioblastoma U-87 MG cell line was obtained from the American Type Culture Collection (Manassas, Va.). U-87 MG cells were cultured in DMEM (Invitrogen Life Technologies, Carlsbad, Calif.) supplemented with 10% fetal bovine serum (Invitrogen Life Technologies, Carlsbad, Calif.) and antibiotics. Cells were routinely passaged by trypsinization and dilution when they reached appropriate confluence. Cells were seeded into 96-well plates (Falcon-Primaria #3872) at a density of about 10,000 cells/well for uses including but not limited to oligomeric compound transfection experiments.

Experiments involving treatment of cells with oligomeric compounds:

When cells reach appropriate confluency, they are treated with oligomeric compounds using a transfection method as described.

LIPOFECTIN™

When cells reached 65-75% confluency, they were treated with oligonucleotide. Oligonucleotide was mixed with LIPOFECTIN™ Invitrogen Life Technologies, Carlsbad, Calif.) in Opti-MEMT-1 reduced serum medium (Invitrogen Life Technologies, Carlsbad, Calif.) to achieve the desired concentration of oligonucleotide and a LIPOFECTIN™ concentration of 2.5 or 3 μg/mL per 100 nM oligonucleotide. This transfection mixture was incubated at room temperature for approximately 0.5 hours. For cells grown in 96-well plates, wells were washed once with 100 μL OPTI-MEMT-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

Other suitable transfection reagents known in the art include, but are not limited to, CYTOFECTIN™, LIPOFECTAMINE™, OLIGOFECTAMINE™, and FUGENE™. Other suitable transfection methods known in the art include, but are not limited to, electroporation.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TₑCₑCₑGTCATCGCTCₑCₑTₑCₑA_{w}GₑGₑGₑ, SEQ ID NO: 1) which is targeted to human H-ras, or ISIS 18078, (GₑTₑGₑCₑGₑCGCGAGCCCGₑAₑAₑAₑTₑCₑ, SEQ ID NO: 2) which is targeted to human Jun-N-terminal kinase-2 (JNK2). Both controls are 2′-O-methoxyethyl gapmers with a phosphorothioate backbone. For mouse or rat cells the positive control oligonucleotide is ISIS 15770 (AₑTₑGₑCₑAₑTTCTGCCCCCAₑAₑGₑGₑAₑ, SEQ ID NO: 3), a 2′-O-methoxyethyl gapmer with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments.

### Example 7

### Analysis of Oligonucleotide Inhibition of a Target Expression

Antisense modulation of a target expression can be assayed in a variety of ways known in the art. For example, a target mRNA levels can be quantitated by, e.g., Northern blot analysis, competitive polymerase chain reaction (PCR), or real-time PCR. Real-time quantitative PCR is presently desired. RNA analysis can be performed on total cellular RNA or poly(A)+ mRNA. One method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 8

### Design of Phenotypic Assays and In Vivo Studies for the Use of Target Inhibitors

Phenotypic Assays

Once target inhibitors have been identified by the methods disclosed herein, the oligomeric compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival (Molecular Probes, Eugene, Oreg.; Perkin Elmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Measurement of the expression of one or more of the genes of the cell after treatment is also used as an indicator of the efficacy or potency of the a target inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

A clinical trial is subjected to rigorous controls to ensure that individuals are not unnecessarily put at risk and that they are fully informed about their role in the study.

To account for the psychological effects of receiving treatments, volunteers are randomly given placebo or a target inhibitor. Furthermore, to prevent the doctors from being biased in treatments, they are not informed as to whether the medication they are administering is a a target inhibitor or a placebo. Using this randomization approach, each volunteer has the same chance of being given either the new treatment or the placebo.

Volunteers receive either the a target inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding a target or a target protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and a target inhibitor treatment. In general, the volunteers treated with placebo have little or no response to treatment, whereas the volunteers treated with the target inhibitor show positive trends in their disease state or condition index at the conclusion of the study.

### Example 9

### RNA Isolation

Poly(A)+ mRNA Isolation

Poly(A)+ mRNA is isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA is isolated using an RNEASY 96™ kit and buffers purchased from Qiagen Inc. (Valencia, Calif.) following the manufacturer's recommended procedures. Briefly, for cells grown on 96-well plates, growth medium is removed from the cells and each well is washed with 200 μL cold PBS. 150 μL Buffer RLT is added to each well and the plate vigorously agitated for 20 seconds. 150 μL of 70% ethanol is then added to each well and the contents mixed by pipetting three times up and down. The samples are then transferred to the RNEASY 96T well plate attached to a QIAVACT manifold fitted with a waste collection tray and attached to a vacuum source. Vacuum is applied for 1 minute. 500 μL of Buffer RW1 is added to each well of the RNEASY 96™ plate and incubated for 15 minutes and the vacuum is again applied for 1 minute. An additional 500 μL of Buffer RW1 is added to each well of the RNEASY 96™ plate and the vacuum is applied for 2 minutes. 1 mL of Buffer RPE is then added to each well of the RNEASY 96™ plate and the vacuum applied for a period of 90 seconds. The Buffer RPE wash is then repeated and the vacuum is applied for an additional 3 minutes. The plate is then removed from the QIAVAC™ manifold and blotted dry on paper towels. The plate is then re-attached to the QIAVAC™ manifold fitted with a collection tube rack containing 1.2 mL collection tubes. RNA is then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 10

### Design and Screening of Duplexed Antisense Compounds

In accordance with the present invention, a series of nucleic acid duplexes comprising the compounds of the present invention and their complements can be designed. The nucleobase sequence of the antisense strand of the duplex comprises at least a portion of an antisense oligonucleotide targeted to a target sequence as described herein. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO: 20) and having a two-nucleobase overhang of deoxythymidine(dT) would have the following structure:

| | |
|---|---|
| cgagaggcggacgggaccgdTdT Antisense Strand | SEQ ID NO:21 |
| \|\|\|\|\|\|\|\|\|\|\|\|\|\|\|\|\|\|\| | |
| dTdTgctctccgcctgccctggc Complement Strand | SEQ ID NO:22 |

In another embodiment, a duplex comprising an antisense strand having the same sequence CGAGAGGCGGACGGGACCG (SEQ ID NO: 20) may be prepared with blunt ends (no single stranded overhang) as shown:

| | |
|---|---|
| cgagaggcggacgggaccg Antisense Strand | SEQ ID NO:20 |
| \|\|\|\|\|\|\|\|\|\|\|\|\|\|\|\|\|\|\| | |
| gctctccgcctgccctggc Complement Strand | SEQ ID NO:23 |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 μM. Once diluted, 30 μL of each strand is combined with 15 μL of a 5× solution of annealing buffer. The final concentration of the buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 μM.

Once prepared, the duplexed compounds are evaluated for their ability to modulate target mRNA levels When cells reach 80% confluency, they are treated with duplexed compounds of the invention. For cells grown in 96-well plates, wells are washed once with 200 μL OPTI-MEM-1™ reduced-serum medium (Gibco BRL) and then treated with 130 μL of OPTI-MEM-1™ containing 5 μg/mL LIPOFECTAMINE 2000™ (Invitrogen Life Technologies, Carlsbad, Calif.) and the duplex antisense compound at the desired final concentration. After about 4 hours of treatment, the medium is replaced with fresh medium. Cells are harvested 16 hours after treatment, at which time RNA is isolated and target reduction measured by quantitative real-time PCR as described herein.

### Example 11

### Real-Time Quantitative PCR Analysis of Target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

RT and PCR reagents were obtained from Invitrogen Life Technologies (Carlsbad, Calif.). RT, real-time PCR was carried out by adding 20 μL PCR cocktail (2.5×PCR buffer minus MgCl₂, 6.6 mM MgCl₂, 375 μM each of DATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by RT, real-time PCR are normalized using either the expression level of GAPDH, a gene whose expression is constant, or by quantifying total RNA using RIBOGREEN™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RIBOGREEN™ are taught in Jones, L. J., et al, (Analytical Biochemistry, 1998, 265, 368-374).

In this assay, 170 μL of RIBOGREEN™ working reagent (RIBOGREEN™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

### Example 12

### Target-Specific Primers and Probes

Probes and primers may be designed to hybridize to a target sequence, using published sequence information.

For example, for human PTEN, the following primer-probe set was designed using published sequence information (GENBANK™ accession number U92436.1, SEQ ID NO: 4).

Forward primer:

(SEQ ID NO:5)

AATGGCTAAGTGAAGATGACAATCAT
Reverse primer:

(SEQ ID NO:6)

TGCACATATCATTACACCAGTTCGT
And
the PCR probe:

(SEQ ID NO:7)

FAM-TTGCAGCAATTCACTGTAAAGCTGGAAAGG-TAMRA,

FAM is the fluorescent dye and TAMRA is the quencher dye.

For example, for human survivin, the following primer-probe set was designed using published sequence information (GENBANK™ accession number NM_{—}001168.1, SEQ ID NO: 8).

Forward primer:

(SEQ ID NO:9)

CACCACTTCCAGGGTTTATTCC
Reverse primer:

(SEQ ID NO:10)

TGATCTCCTTTCCTAAGACATTGCT
And
the PCR probe:

(SEQ ID NO:11)

FAM-ACCAGCCTTCCTGTGGGCCCCT-TAMRA,

where FAM is the fluorescent dye and TAMRA is the quencher dye.

For example, for human eIF4E, the following primer-probe set was designed using published sequence information (GENBANK™ accession number M15353.1, SEQ ID NO: 12).

Forward primer:

(SEQ ID NO:13)

TGGCGACTGTCGAACCG
Reverse primer:

(SEQ ID NO:14)

AGATTCCGTTTTCTCCTCTTCTGTAG
And
the PCR probe:

(SEQ ID NO:15)

FAM-AAACCACCCCTACTCCTAATCCCCCG-TAMRA,

where FAM is the fluorescent dye and TAMRA is the quencher dye.

For example, for mouse eIF4E, the following primer-probe set was designed using published sequence information (GENBANK™ accession number NM_{—}007917.2, SEQ ID NO: 16).

Forward primer:

(SEQ ID NO:17)

AGGACGGTGGCTGATCACA
Reverse primer:

(SEQ ID NO:18)

TCTCTAGCCAGAAGCGATCGA
And
the PCR probe:

(SEQ ID NO:19)

FAM-TGAACAAGCAGCAGAGACGGAGTGA-TAMRA,

where FAM is the fluorescent dye and TAMRA is the quencher dye.

### Example 13

### Northern Blot Analysis of a Target mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human a target, a human a target specific primer probe set is prepared by PCR. To normalize for variations in loading and transfer efficiency membranes are stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 14

### Western Blot Analysis of Target Protein Levels

Western blot analysis (immunoblot analysis) is carried out using standard methods. Cells are harvested 16-20 h after oligonucleotide treatment, washed once with PBS, suspended in Laemmli buffer (100 μl/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

### Example 15

### In Vitro Assay of Selected Differentially Modified siRNAs

Differentially modified siRNA duplexes designed to target human survivin using published sequence information were prepared and assayed as described below. The antisense strand was held constant as a 4′-thio gapped strand and 3 different sense strands were compared. The nucleosides are annotated as to chemical modification as per the legend at the beginning of the examples.

| SEQ ID | | |
|---|---|---|
| NO./ | | |
| ISIS NO. | Composition (5′ 3′) | Features |
| 24/353537 | UₜUₜUₜGAAAAUGUUGAUCUₜCₜCₜ | 4′-S wings |
| (as) | | (3/13/3) |
| 25/352512 | GₘGₘAₘGₘAₘUₘCₘAₘAₘCₘAₘ | 2′-OCH₃full |
| (s) | UₘUₘUₘUₘCₘAₘAₘAₘ | |
| 25/352513 | GGₘAₘGₘAₘUₘCₘAₘAₘCₘAₘUₘ | 2′-OCH₃block |
| (s) | UₘUₘUₘCₘAₘAₘA | (1/17/1) |
| 25/352514 | GGₑAGₑAUₑCAₑACₑAUₑUUₑUCₑ | MOE |
| (s) | AAₑA | alternating |

The differentially modified siRNA duplexes were assayed for their ability to inhibit target mRNA levels in HeLa cells. Culture methods used for HeLa cells are available from the ATCC and may be found, for example, at www (dot)atcc.org. For cells grown in 96-well plates, wells were washed once with 200 μL OPTI-MEM-1 reduced-serum medium and then treated with 130 μL of OPTI-MEM-1 containing 12 μg/mL LIPOFECTIN™ (Invitrogen Life Technologies, Carlsbad, Calif.) and the dsRNA at the desired concentrations. After about 5 hours of treatment, the medium was replaced with fresh medium. Cells were harvested 16 hours after treatment, at which time RNA was isolated and target reduction measured by RT-PCR as previously described. Dose-response data was used to determine the IC50 for each pair noted below (antisense:sense).

| Construct | Assay/Species | Target | IC50 (nM) |
|---|---|---|---|
| 353537:352512 | Dose Response/Human Survivin | 0.60192 | |
| 353537:352513 | Dose Response/Human Survivin | 0.71193 | |
| 353537:352514 | Dose Response/Human Survivin | 0.48819. | |

### Example 16

### In Vitro Assay of Differentially Modified siRNAs having MOE Modified Sense and 4′-thio (4′-thio/2′-OCH₃) Gapmer Antisense Strands

In accordance with the present invention, a series of oligomeric compounds were synthesized and tested for their ability to reduce target expression over a range of doses relative to an unmodified compound. The compounds tested were 19 nucleotides in length having phosphorothioate internucleoside linkages throughout.

HeLa cells were treated with the double stranded oligomeric compounds (siRNA constructs) shown below (antisense strand followed by the sense strand of the duplex) at concentrations of 0, 0.15, 1.5, 15, and 150 nM using methods described herein. The nucleosides are annotated as to chemical modification as per the legend at the beginning of the examples. Expression levels of human PTEN were determined by quantitative real-time PCR and normalized to RIBOGREEN™ as described in other examples herein. Resulting dose-response curves were used to determine the IC50 for each pair. Also shown is the effect of each duplex on target mRNA levels as a percentage of untreated control (% UTC).

| | | | |
|---|---|---|---|
| SEQ ID | | | |
| NO./ | | | % |
| ISIS NO. | Composition (5′ to 3′) | IC50 | UTC |

| | | | |
|---|---|---|---|
| 26/xxxxxx | UUGUCUCUGGUCCUUACUU | 0.94 | 13 |
| (as) | | | |
| 27/xxxxxx | AAGUAAGGACCAGAGACAA | | |
| (s) | | | |
| 26/xxxxxx | UUGUCUCUGGUCCUUACUU | .055 | 13 |
| (as) | | | |
| 27/359351 | AₑAₑGₑUAAGGACCAGAGACₑAₑAₑ | | |
| (s) | | | |
| 26/359347 | UₜUₜGUCUCUGGUCCUUACUₜUₜ | 2.2 | 25 |
| (as) | | | |
| 27/359551 | AₑAₑGₑUAAGGACCAGAGACₑAₑAₑ | | |
| (s) | | | |
| 26/359346 | UₜUₜGUCUCUGGUCCUUACₘUₘUₘ | 0.18 | 11 |
| (as) | | | |
| 27/359351 | AₑAₑGₑUAAGGACCAGAGACₑAₑAₑ | | |
| (s) | | | |
| 26/359345 | UₜUₜGUCUCUGGUCCUUACUₜUₜ | 5.3 | 18 |
| (as) | | | |
| 27/xxxxxx | AAGUAAGGACCAGAGACAA | | |
| (s) | | | |
| 26/359346 | UₜUₜGUCUCUGGUCCUUACₘUₘUₘ | 0.73 | 15 |
| (as) | | | |
| 27/xxxxxx | AAGUAAGGACCAGAGACAA | | |
| (s) | | | |
| 26/359345 | UₜUₜGUCUCUGGUCCUUACUₜUₜ | 0.49 | 14 |
| (as) | | | |
| 27/xxxxx | AAₑGUₑAAₑGGₑACₑCAₑGAₑGAₑCAₑA | | |
| (s) | | | |
| 26/359345 | UₜUₜGUCUCUGGUCCUUACUₜUₜ | 0.55 | 15 |
| (as) | | | |
| 27/359351 | AₑAₑGₑUAAGGACCAGAGACₑAₑAₑ | | |
| (s) | | | |

From these data it is evident that the activity of the double strand construct containing the 4′-thio gapmer RNA in the antisense strand paired with an RNA sense strand (359345_{—}341401 having an IC50 of 5.3) can be improved by incorporating 2'MOE modifications into the sense strand on the terminal ends or in an alternating configuration with RNA. It is also evident that improvements in IC50 values can be obtained over the unmodified pure RNA construct (341391_{—}341401; RNA in both strands with an IC50 value of 0.94) by using an alternating motif.

### Example 17

### In Vitro Assay of Selected Differentially Modified siRNAs

Selected siRNAs (shown below as antisense strand followed by the sense strand of the duplex) were prepared and evaluated in HeLa cells treated as described herein with varying doses of the selected siRNAs. The mRNA levels were quantitated using real-time PCR as described herein and were compared to untreated control levels (% UTC). The IC50's were calculated using the linear regression equation generated by plotting the normalized mRNA levels to the log of the concentrations used.

| | | | |
|---|---|---|---|
| SEQ ID | | | |
| NO./ | | | % |
| ISIS NO. | Composition (5′ to 3′) | IC50 | UTC |

| | | | |
|---|---|---|---|
| 26/359346 | UₜUₜGUCUCUGGUCCUUACₘUₘUₘ | 1.9 | 10 |
| (as) | | | |
| 27/367287 | AAGUₜAAGGACₜCₜAGAGACₜAA | | |
| (s) | | | |
| 26/359345 | UₜUₜGUCUCUGGUCCUUACUₜUₜ | 1.7 | 20 |
| (as) | | | |
| 27/367287 | AAGUₜAAGGACₜCₜAGAGACₜAA | | |
| (s) | | | |
| 26/359345 | UₜUₜGUCUCUGGUCCUUACUₜUₜ | 0.2 | 10 |
| (as) | | | |
| 27/367288 | AₜAₜGUAAGGACCAGAGACAₜAₜ | | |
| (s) | | | |
| 26/359346 | UₜUₜGUCUCUGGUCCUUACₘUₘUₘ | <0.1 | 10 |
| (as) | | | |
| 27/367288 | AₜAₜGUAAGGACCAGAGACAₜAₜ | | |
| (s) | | | |
| 26/359345 | UₜUₜGUCUCUGGUCCUUACUₜUₜ | 0.5 | 15 |
| (as) | | | |
| 27/359351 | AₑAₑGₑUAAGGACCAGAGACₑAₑAₑ | | |
| (s) | | | |
| 26/359346 | UₜUₜGUCUCUGGUCCUUACₘUₘUₘ | 0.2 | 11 |
| (as) | | | |
| 27/359351 | AₑAₑGₑUAAGGACCAGAGACₑAₑAₑ | | |
| (s) | | | |
| 26/359995 | UₘU_{f}GₘU_{f}CₘU_{f}CₘU_{f}GₘG_{f}UₘC_{f}CₘU_{f}UₘA_{f} | 0.4 | 17 |
| (as) | CₘU_{f}Uₘ | | |
| 27/359351 | AₑAₑGₑUAAGGACCAGAGACₑAₑAₑ | | |
| (s) | | | |
| 26/359345 | UₜUₜGUCUCUGGUCCUUACUₜUₜ | 0.2 | 13 |
| (as) | | | |
| 27/359996 | AₘA_{f}GₘU_{f}AₘA_{f}GₘG_{f}AₘC_{f}CₘA_{f}GₘA_{f}GₘA_{f} | | |
| (s) | CₘA_{f}Aₘ | | |
| 26/359346 | UₜUₜGUCUCUGGUCCUUACₘUₘUₘ | 0.2 | 13 |
| (as) | | | |
| 27/359996 | AₘA_{f}GₘU_{f}AₘA_{f}GₘG_{f}AₘC_{f}CₘA_{f}GₘA_{f}GₘA_{f} | | |
| (s) | CₘA_{f}Aₘ | | |
| 26/361203 | UUGₘUCUCUₘGGUCCₘUUACUₘU | <0.1 | — |
| (as) | | | |
| 27/359351 | AₑAₑGₑUAAGGACCAGAGACₑAₑAₑ | | |
| (s) | | | |
| 26/361209 | UUGUₘCUCUGₘGUCCUₘUACUUₘ | 1.5 | — |
| (as) | | | |
| 27/359351 | AₑAₑGₑUAAGGACCAGAGACₑAₑAₑ | | |
| (s) | | | |
| 26/361204 | UUGUₑCUCUGGₑUCCUUACUₑU | 1.5 | — |
| (as) | | | |
| 27/359351 | AₑAₑGₑUAAGGACCAGAGACₑAₑAₑ | | |
| (s) | | | |
| 26/361205 | UUGUCₑUCUGGUCₑCUUACₑUₑUₑ | 2.5 | — |
| (as) | | | |
| 27/359351 | AₑAₑGₑUAAGGACCAGAGACₑAₑAₑ | | |
| (s) | | | |
| 26/361206 | UUGUCₑUₑCUGGUₑCₑCUUACUₑUₑ | — | — |
| (as) | | | |
| 27/359351 | AₑAₑGₑUAAGGACCAGAGACₑAₑAₑ | | |
| (s) | | | |
| 26/361207 | UUGUCUₑCₑUGGₑUₑCCUUACₑUₑUₑ | 10.1 | — |
| (as) | | | |
| 27/359351 | AₑAₑGₑUAAGGACCAGAGACₑAₑAₑ | | |
| (s) | | | |
| 26/341391 | UUGUCUCUGGUCCUUACUU | 0.1 | — |
| (as) | | | |
| 27/341401 | AAGUAAGGACCAGAGACAA | | |
| (s) | | | |
| 26/359979 | UUGUCₘUCUₘGGUₘCCUₘUACₘUₘUₘ | — | — |
| (as) | | | |
| 27/359351 | AₑAₑGₑUAAGGACCAGAGACₑAₑAₑ | | |
| (s) | | | |
| 26/359980 | UUGUCUₘCₘUGGₘUₘCCUUACₘUₘUₘ | 0.2 | — |
| (as) | | | |
| 27/359351 | AₑAₑGₑUAAGGACCAGAGACₑAₑAₑ | | |
| (s) | | | |
| 26/359980 | UUGUCUₘCₘUGGₘUₘCCUUACₘUₘUₘ | 0.1 | — |
| (as) | | | |
| 27/361221 | AₘAₘGₘUAAGGACCAGAGACₘAₘAₘ | | |
| (s) | | | |

### Example 18

### In Vitro Assay of Modified siRNAs Targeted to Human Survivin

In accordance with the present invention, a series of oligomeric compounds were synthesized and tested for their ability to reduce survivin expression over a range of doses. HeLa cells were treated with the double stranded oligomeric compounds (siRNA constructs) shown below (antisense strand followed by the sense strand of the duplex) at concentrations of 0.0006 nM, 0.084 nM, 0.16 nM, 0.8 nM, 4 nM, or 20 nM using methods described herein. The nucleosides are annotated as to chemical modification as per the legend at the beginning of the examples. Expression levels of human survivin were determined using real-time PCR methods as described herein. The effect of the 20 nM dose on survivin mRNA levels is shown below. Results are presented as a percentage of untreated control mRNA levels.

| | | |
|---|---|---|
| SEQ ID | | |
| NO./ | | |
| ISIS NO. | Composition (5′ to 3′) | % UTC |

| | | |
|---|---|---|
| 24/343867 | UUUGAAAAUGUUGAUCUCC | 3 |
| (as) | | |
| 25/343868 | GGAGAUCAACAUUUUCAAA | |
| (s) | | |
| 24/352506 | UUUGAAₘAₘAUGₘUₘUGAUCUₘCₘCₘ | 2 |
| (as) | | |
| 25/371314 | GₑGₑAₑGₑAₑUCAACAUUUUₑCₑAₑAₑAₑ | |
| (s) | | |
| 24/352506 | UUUGAAₘAₘAUGₘUₘUGAUCUₘCₘCₘ | 3 |
| (as) | | |
| 25/371316 | GₘGₘAₘGAUCAACAUUUUCAₘAₘAₘ | |
| (s) | | |
| 24/352506 | UUUGAAₘAₘAUGₘUₘUGAUCUₘCₘCₘ | 2 |
| (as) | | |
| 25/371313 | GₑGₑAₑGAUCAACAUUUUCAₑAₑAₑ | |
| (s) | | |
| 24/353537 | UₜUₜUₜGAAAAUGUUGAUCUₜCₜCₜ | 5 |
| (as) | | |
| 25/371313 | GₑGₑAₑGAUCAACAUUUUCAₑAₑAₑ | |
| (s) | | |
| 24/353537 | UₜUₜUₜGAAAAUGUUGAUCUₜCₜCₜ | 5 |
| (as) | | |
| 25/352514 | GGₑAGₑAUₑCAₑACₑAUₑUUₑUCₑAAₑA | |
| (s) | | |
| 24/353537 | UₜUₜUₜGAAAAUGUUGAUCUₜCₜCₜ | 6 |
| (as) | | |
| 25/371314 | GₑGₑAₑGₑAₑUCAACAUUUUₑCₑAₑAₑAₑ | |
| (s) | | |
| 24/353537 | UₜUₜUₜGAAAAUGUUGAUCUₜCₜCₜ | 5 |
| (as) | | |
| 25/371315 | GₑGₑAₑGAUCAACₑAₑUUUUCAₑAₑAₑ | |
| (s) | | |
| 24/353537 | UₜUₜUₜGAAAAUGUUGAUCUₜCₜCₜ | 5 |
| (as) | | |
| 25/371316 | GₘGₘAₘGAUCAACAUUUUCAₘAₘAₘ | |
| (s) | | |
| 24/353540 | UₘUₘUₘGAAAAUGUUGAUCUₜCₜCₜ | 3 |
| (as) | | |
| 25/371313 | GₑGₑAₑGAUCAACAUUUUCAₑAₑAₑ | |
| (s) | | |
| 24/353540 | UₘUₘUₘGAAAAUGUUGAUCUₜCₜCₜ | 2 |
| (as) | | |
| 25/352514 | GGₑAGₑAUₑCAₑACₑAUₑUUₑUCₑAAₑA | |
| (s) | | |
| 24/353540 | UₘUₘUₘGAAAAUGUUGAUCUₜCₜCₜ | 3 |
| (as) | | |
| 25/371314 | GₑGₑAₑGₑAₑUCAACAUUUUₑCₑAₑAₑAₑ | |
| (s) | | |
| 24/353540 | UₘUₘUₘGAAAAUGUUGAUCUₜCₜCₜ | 3 |
| (as) | | |
| 25/371315 | GₑGₑAₑGAUCAACₑAₑUUUUCAₑAₑAₑ | |
| (s) | | |
| 24/353540 | UₘUₘUₘGAAAAUGUUGAUCUₜCₜCₜ | |
| (as) | | |
| 25/371316 | GₘGₘAₘGAUCAACAUUUUCAₘAₘAₘ | |
| (s) | | |
| 24/368679 | UₘU_{f}UₘG_{f}AₘA_{f}AₘA_{f}UₘG_{f}UₘU_{f}GₘA_{f}UₘC_{f}Uₘ | 2 |
| (as) | C_{f}Cₘ | |
| 25/371313 | GₑGₑAₑGAUCAACAUUUUCAₑAₑAₑ | |
| (s) | | |
| 24/368679 | UₘU_{f}UₘG_{f}AₘA_{f}AₘA_{f}UₘG_{f}UₘU_{f}GₘA_{f}UₘC_{f}Uₘ | 3 |
| (as) | C_{f}Cₘ | |
| 25/371314 | GₑGₑAₑGₑAₑUCAACAUUUUₑCₑAₑAₑAₑ | |
| (s) | | |
| 24/368679 | UₘU_{f}UₘG_{f}AₘA_{f}AₘA_{f}UₘG_{f}UₘU_{f}GₘA_{f}UₘC_{f}Uₘ | 3 |
| (as) | C_{f}Cₘ | |
| 25/371316 | GₘGₘAₘGAUCAACAUUUUCAₘAₘAₘ | |
| (s) | | |
| 24/352506 | UUUGAAₘAₘAUGₘUₘUGAUCUₘCₘCₘ | 12 |
| (as) | | |
| 25/352514 | GGₑAGₑAUₑCAₑACₑAUₑUUₑUCₑAAₑA | |
| (s) | | |
| 24/368679 | UₘU_{f}UₘG_{f}AₘA_{f}AₘA_{f}UₘG_{f}UₘU_{f}GₘA_{f}UₘC_{f}Uₘ | 8 |
| (as) | C_{f}Cₘ | |
| 25/371315 | GₑGₑAₑGAUCAACₑAUUUUCAₑAₑAₑ | |
| (s) | | |

### Example 19

### In Vitro Assay of Selected Differentially Modified siRNAs Targeted to Human eIF4E

In accordance with the present invention, a series of oligomeric compounds were synthesized and tested for their ability to reduce eIF4E expression over a range of doses. The nucleosides are annotated as to chemical modification as per the legend at the beginning of the examples. HeLa cells were treated with the double stranded oligomeric compounds (siRNA constructs) shown below (antisense strand followed by the sense strand to which it was duplexed) at concentrations of 0.0006 nM, 0.032 nM, 0.16 nM, 0.8 nM, 4 nM, or 20 nM using methods described herein. Expression levels of human eIF4E were determined using real-time PCR methods as described herein. Resulting dose-response curves were used to determine the IC50 for each pair as shown below.

| SEQ ID | | |
|---|---|---|
| NO./ | | |
| ISIS NO. | Composition (5′ to 3′) | IC50 |
| 30/371286 | UUUAGCUCUAACAUUAACA | 0.440 |
| (as) | | |
| 31/371280 | UGUUAAUGUUAGAGCUAAA | |
| (s) | | |
| 30/371287 | UUUAGCₘUₘCUAₘAₘCAUUAAₘCₘAₘ | 0.356 |
| (as) | | |
| 31/371280 | UGUUAAUGUUAGAGCUAAA | |
| (s) | | |
| 30/371287 | UUUAGCₘUₘCUAₘAₘCAUUAAₘCₘAₘ | 2.520 |
| (as) | | |
| 31/371284 | UₑGₑUₑUAAUGUUAGAGCUAₑAₑAₑ | |
| (s) | | |
| 32/371297 | UUACUAGACAACUGGAUAU | 0.381 |
| (as) | | |
| 33/371291 | AUAUCCAGUUGUCUAGUAA | |
| (s) | | |
| 32/371298 | UUACUAₘGₘACAₘAₘCUGGAUₘAₘUₘ | 0.260 |
| (as) | | |
| 33/371291 | AUAUCCAGUUGUCUAGUAA | |
| (s) | | |
| 32/371298 | UUACUAₘGₘACAₘAₘCUGGAUₘAₘUₘ | 0.260 |
| (as) | | |
| 33/371295 | AₑUₑAₑUCCAGUUGUCUAGUₑAₑAₑ | |
| (s) | | |
| 32/379960 | UₘU_{f}AₘC_{f}UₘA_{f}GₘA_{f}CₘA_{f}AₘC_{f}UₘG_{f}GₘA_{f}Uₘ | 0.260 |
| (as) | A_{f}Uₘ | |
| 33/371295 | AₑUₑAₑUCCAGUUGUCUAGUₑAₑAₑ | |
| (s) | | |
| 34/371308 | UUAAAAAGUGAGUAGUCAC | 0.126 |
| (as) | | |
| 35/371302 | GUGACUACUCACUUUUUAA | |
| (s) | | |
| 34/371309 | UUAAAAₘAₘGUGₘAₘGUAGUCₘAₘCₘ | 0.168 |
| (as) | | |
| 35/371302 | GUGACUACUCACUUUUUAA | |
| (s) | | |
| 34/371309 | UUAAAAₘAₘGUGₘAₘGUAGUCₘAₘCₘ | 0.040 |
| (as) | | |
| 35/371306 | GₑUₑGₑACUACUCACUUUUUₑAₑAₑ | |
| (s) | | |
| 34/371309 | UUAAAAₘAₘGUGₘAₘGUAGUCₘAₘCₘ | 0.017 |
| (as) | | |
| 35/379965 | GₘU_{f}GₘA_{f}CₘU_{f}AₘC_{f}UₘC_{f}AₘC_{f}UₘU_{f}UₘU_{f}Uₘ | |
| (s) | A_{f}Aₘ | |

### Example 20

### In Vitro Assay of Selected Differentially Modified siRNAs Targeted to Mouse eIF4E

In accordance with the present invention, a series of oligomeric compounds were synthesized and tested for their ability to reduce eIF4E expression over a range of doses. The nucleosides are annotated as to chemical modification as per the legend at the beginning of the examples. b.END cells were treated with the double stranded oligomeric compounds (siRNA constructs) shown below (antisense strand followed by the sense strand of the duplex) at concentrations of 0.0625 nM, 0.25 nM, 1 nM, or 4 nM using methods described herein. Expression levels of mouse eIF4E were determined using real-time PCR methods as described herein. Resulting dose-response curves were used to determine the IC50 for each pair as shown below.

| | | |
|---|---|---|
| SEQ ID | | |
| NO./ | | |
| ISIS NO. | Composition (5′ to 3′) | IC50 |

| | | |
|---|---|---|
| 30/371286 | UUUAGCUCUAACAUUAACA | 0.2055 |
| (as) | | |
| 31/371280 | UGUUAAUGUUAGAGCUAAA | |
| (s) | | |
| 30/371287 | UUUAGCₘUₘCUAₘAₘCAUUAAₘCₘAₘ | 0.238 |
| (as) | | |
| 31/371280 | UGUUAAUGUUAGAGCUAAA | |
| (s) | | |
| 30/371287 | UUUAGCₘUₘCUAₘAₘCAUUAAₘCₘAₘ | 9.496 |
| (as) | | |
| 31/371284 | UₑGₑUₑUAAUGUUAGAGCUAₑAₑAₑ | |
| (s) | | |
| 30/371286 | UUUAGCUCUAACAUUAACA | 1.193 |
| (as) | | |
| 31/371284 | UₑGₑUₑUAAUGUUAGAGCUAₑAₑAₑ | |
| (s) | | |
| 32/371297 | UUACUAGACAACUGGAUAU | 0.1859 |
| (as) | | |
| 33/371291 | AUAUCCAGUUGUCUAGUAA | |
| (s) | | |
| 32/371298 | UUACUAₘGₘACAₘAₘCUGGAUₘAₘUₘ | 0.1946 |
| (as) | | |
| 33/371291 | AUAUCCAGUUGUCUAGUAA | |
| (s) | | |
| 32/371297 | UUACUAGACAACUGGAUAU | 0.0936 |
| (as) | | |
| 33/371295 | AₑUₑAₑUCCAGUUGUCUAGUₑAₑAₑ | |
| (s) | | |
| 32/371298 | UUACUAₘGₘACAₘAₘCUGGAUₘAₘUₘ | 0.1151 |
| (as) | | |
| 33/371295 | AₑUₑAₑUCCAGUUGUCUAGUₑAₑAₑ | |
| (s) | | |
| 34/371308 | UUAAAAAGUGAGUAGUCAC | 0.2926 |
| (as) | | |
| 35/371302 | GUGACUACUCACUUUUUAA | |
| (s) | | |
| 34/371309 | UUAAAAₘAₘGUGₘAₘGUAGUCₘAₘCₘ | 0.1626 |
| (as) | | |
| 35/371302 | GUGACUACUCACUUUUUAA | |
| (s) | | |
| 34/371308 | UUAAAAAGUGAGUAGUCAC | 0.0632 |
| (as) | | |
| 35/371306 | GₑUₑGₑACUACUCACUUUUUₑAₑAₑ | |
| (s) | | |
| 34/371309 | UUAAAAₘAₘGUGₘAₘGUAGUCₘAₘCₘ | 0.0061 |
| (as) | | |
| 35/371306 | GₑUₑGₑACUACUCACUUUUUₑAₑAₑ. | |
| (s) | | |

### Example 21

### Blockmer Walk of 5 2′-O-methy Modified Nucleosides in the Antisense Strand of siRNAs Assayed for PTEN mRNA Levels Against Untreated Control

The antisense (AS) strands listed below were designed to target human PTEN, and each was duplexed with the same sense strand (ISIS 271790, shown below). The duplexes were tested for their ability to reduce PTEN expression over a range of doses to determine the relative positional effect of the 5 modifications using methods described herein. The nucleosides are annotated as to chemical modification as per the legend at the beginning of the examples. Expression levels of PTEN were determined using real-time PCR methods as described herein, and were compared to levels determined for untreated controls.

| SEQ ID NO:/ | |
|---|---|
| ISIS NO | Sequence 5′-3′ |
| 36/271790 (S) | CAAAUCCAGAGGCUAGCAGdTdT |
| 37/271071 (AS) | CₘUₘGₘCₘUₘAGCCUCUGGAUUUGdTdT |
| 37/271072 (AS) | CUₘGₘCₘUₘAₘGCCUCUGGAUUUGdTdT |
| 37/271073 (AS) | CUGₘCₘUₘAₘGₘCCUCUGGAUUUGdTdT |
| 37/271074 (AS) | CUGCₘUₘAₘGₘCₘCUCUGGAUUUGdTdT |
| 37/271075 (AS) | CUGCUₘAₘGₘCₘCₘUCUGGAUUUGdTdT |

The siRNAs having 2′-O-methyl groups at least 2 positions removed from the siRNAs having 5,2′-O-methyl groups at least 2 positions removed from the 5′-end of the antisense strand reduced PTEN mRNA levels to from 25 to 35% of untreated control. The remaining 2 constructs increased PTEN mRNA levels above untreated control.

### Example 22

### Solid Block of 2′-O-methyl Modified Nucleosides in the Antisense Strand of siRNAs Assayed for PTEN mRNA Levels Against Untreated Control

The antisense (AS) strands listed below were designed to target human PTEN, and each was duplexed with the same sense strand 271790. The duplexes were tested for their ability to reduce PTEN expression over a range of doses to determine the relative effect of adding either 9 or 14, 2′-O-methyl modified nucleosides at the 3′-end of the resulting siRNAs. The nucleosides are annotated as to chemical modification as per the legend at the beginning of the examples. Expression levels of PTEN were determined using real-time PCR methods as described herein, and were compared to levels determined for untreated controls.

| SEQ ID NO:/ | |
|---|---|
| ISIS NO | Sequence 5′-3′ |
| 36/271790 (S) | CAAAUCCAGAGGCUAGCAGdTdT |
| 37/271079 (AS) | CUGCUAGCCUCUGₘGₘAₘUₘUₘUₘGₘUₘUₘ |
| 37/271081 (AS) | CUGCUAGCₘCₘUₘCₘUₘGₘGₘAₘUₘUₘUₘGₘUₘUₘ |

The siRNA having 9,2′-O-methyl nucleosides reduced PTEN mRNA levels to about 40% of untreated control whereas the construct having 14, 2′-O-methyl nucleosides only reduced PTEN mRNA levels to about 98% of control.

### Example 23

### 2′-O-methy Blockmers (siRNA vs asRNA)

A series of blockmers were prepared as single strand antisense RNAs (asRNAs). The antisense (AS) strands listed below were designed to target PTEN, and each was also assayed as part of a duplex with the same sense strand (ISIS 308746, shown below) for their ability to reduce PTEN expression levels. T24 cells were treated with the single stranded or double stranded oligomeric compounds created with the antisense compounds shown below using methods described herein. The nucleosides are annotated as to chemical modification as per the legend at the beginning of the examples. Expression levels of human PTEN were determined using real-time PCR methods as described herein, and were compared to levels determined for untreated controls.

| SEQ ID NO:/ | |
|---|---|
| ISIS NO | Sequence 5′-3′ |
| 39/308746 (S) | AAGUAAGGACCAGAGACAAA |
| 40/303912 (AS) | P-UUUGUCUCUGGUCCUUACUU |
| 40/316449 (AS) | P-UUUGUCUCUGGUCCUUACₘUₘUₘ |
| 40/335223 (AS) | P-UUUGUCUCUGGUCCUₘUₘAₘCUU |
| 40/335224 (AS) | P-UUUGUCUCUGGUₘCₘCₘUUACUU |
| 40/335225 (AS) | P-UUUGUCUCUₘGₘGₘUCCUUACUU |
| 40/335226 (AS) | P-UUUGUCₘUₘCₘUGGUCCUUACUU |
| 40/335227 (AS) | P-UUUₘGₘUₘCUCUGGUCCUUACUU |
| 40/335228 (AS) | P-UₘUₘUₘGUCUCUGGUCCUUACUU |

All of the asRNAs and siRNAs showed activity with the asRNAs having better activity than the corresponding duplex in each case. A clear dose response was seen for all of the siRNA constructs (20, 40, 80 and 150 nm doses). A dose-responsive effect was also observed for the asRNAs for 50, 100 and 200 nm doses. In general the siRNAs were more active in this system at lower doses than the asRNAs and at the 150 nm dose were able to reduce PTEN mRNA levels to from 15 to 40% of untreated control. The duplex containing unmodified 303912 reduced PTEN mRNA levels to about 19% of the untreated control.

### Example 24

### siRNA Hemimer Constructs

Three siRNA hemimer constructs were prepared and were tested for their ability to reduce PTEN expression levels. The hemimer constructs had 7,2′-O-methyl nucleosides at the 3′-end. The hemimer was put in the sense strand only, the antisense strand only and in both strands to compare the effects. Cells were treated with the double stranded oligomeric compounds (siRNA constructs) shown below (antisense strand followed by the sense strand of the duplex) using methods described herein. The nucleosides are annotated as to chemical modification as per the legend at the beginning of the examples. Expression levels of PTEN were determined using real-time PCR methods as described herein, and were compared to levels determined for untreated controls.

| SEQ ID NO:/ | |
|---|---|
| ISIS NO | Constructs (overhangs) 5′-3′ |
| 38/XXXXX (AS) | CUGCUAGCCUCUGGAₘUₘUₘUₘGₘUₘUₘ |
| 41/271068 (S) | CAAAUCCAGAGGCUAₘGₘCₘAₘGₘUₘUₘ |
| 38/XXXXX (AS) | CUGCUAGCCUCUGGAUUUGUU |
| 41/271068 (S) | CAAAUCCAGAGGCUAₘGₘCₘAₘGₘUₘUₘ |
| 38/XXXXX (AS) | CUGCUAGCCUCUGGAₘUₘUₘUₘGₘUₘUₘ |
| 41/XXXXX (S) | CAAAUCCAGAGGCUAGCAGUU |

The construct having the 7,2′-O-methyl nucleosides only in the antisense strand reduced PTEN mRNA levels to about 23% of untreated control. The construct having the 7,2′-O-methyl nucleosides in both strands reduced the PTEN mRNA levels to about 25% of untreated control. When the 7,2′-O-methyl nucleosides were only in the sense strand, PTEN mRNA levels were reduced to about 31% of untreated control.

### Example 25

### Representative siRNAs Prepared having 2'O-Me Gapmers

The following antisense strands of selected siRNA duplexes targeting PTEN are hybridized to their complementary full phosphodiester sense strands. Activity is measured using methods described herein. The nucleosides are annotated as to chemical modification as per the legend at the beginning of the examples.

| | |
|---|---|
| SEQ ID NO: | Sequence (5′-3′) |

| | |
|---|---|
| 42/300852 | CUGCₘUₘAₘGₘCCUCUGGAUUₘUₘGₘAₘ |
| 42/300853 | P-CUGCₘUₘAₘGₘCCUCUGGAUUₘUₘGₘAₘ |
| 42/300854 | CₘUₘGₘCₘUAGCCUCUGGAUUₘUₘGₘAₘ |
| 42/300855 | P-CUGCₘUₘAₘGₘCCUCUGGAUUₘUₘGₘAₘ |
| 42/300856 | CₘUₘAₘGₘCCUCUGGAUUₘUₘGₘAₘ |
| 42/300858 | CUGCₘUₘAₘGₘCCUCUGGAUUₘUₘGₘAₘ |
| 42/300859 | P-CUGCₘUₘAₘGₘCCUCUGGAUUₘUₘGₘAₘ |
| 42/300860 | CₘUₘAₘGₘCCUCUGGAUUₘUₘGₘAₘ |
| 43/303913 | GₘUₘCₘUₘCUGGUCCUUAₘCₘUₘUₘ |
| 44/303915 | UₘUₘUₘUₘGUCUCUGGUCₘCₘUₘUₘ |
| 45/303917 | CₘUₘGₘGₘUCCUUACUUCₘCₘCₘCₘ |
| 46/308743 | P-UₘUₘUₘGUCUCUGGUCCUUACₘUₘUₘ |
| 47/308744 | P-UₘCₘUₘCₘUₘGGUCCUUACUUₘCₘCₘCₘCₘ |
| 46/328795 | P-UUUGₘUₘCₘUₘCUGGUCCUUAₘCₘUₘUₘ. |

### Example 26

### Representative siRNAs Prepared Having 2′-F Modified Nucleosides and Various Structural Motifs

The following antisense strands of siRNAs targeting PTEN were tested as single strands alone or were hybridized to their complementary full phosphodiester sense strand and were tested in duplex. The nucleosides are annotated as to chemical modification as per the legend at the beginning of the examples. Bolded and italicized “C” indicates a 5-methyl C ribonucleoside.

| | |
|---|---|
| SEQ ID | |
| NO/ | |
| ISIS NO | Sequences 5′-3′ |

| | |
|---|---|
| 40/319022 | U_{f}U_{f}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f} |
| AS | |
| 40/333749 | UUUGUCUCUGGUCCU_{f}U_{f}A_{f}CUU |
| AS | |
| 40/333750 | UUUGUCUCUGGU_{f}C_{f}C_{f}UUACUU |
| AS | |
| 40/333751 | UUUGUCUCUGGU_{f}C_{f}C_{f}UUACUU |
| AS | |
| 40/333752 | UUUGUC_{f}U_{f}C_{f}UGGUCCUUACUU |
| AS | |
| 40/333753 | UUU_{f}G_{f}U_{f}CUCUGGUCCUUACUU |
| AS | |
| 40/333754 | U_{f}U_{f}U_{f}GUCUCUGGUCCUUACUU |
| AS | |
| 40/333756 | UUUGUCUCUGGUCCUUAC_{f}U_{f}U_{f} |
| AS | |
| 40/334253 | UUUGUCUCU_{f}G_{f}G_{f}UCCUUACUU |
| AS | |
| 40/334254 | UUUGUCUCUGGUCCUU_{f}A_{f}C_{f}U_{f}U_{f} |
| AS | |
| 40/334255 | UUU_{f}G_{f}U_{f}CUCUGGUCCUUACUU |
| AS | |
| 40/334256 | UUU_{f}G_{f}U_{f}CUCUGGU_{f}C_{f}C_{f}UUACUU |
| AS | |
| 40/334257 | U_{f}U_{f}U_{f}GUCUCUGGUCCUUACUU |
| AS | |
| 40/317466 | U_{f}U_{f}U_{f}GUCUCUGGUCCUUAC_{f}U_{f}U |
| AS | |
| 40/317468 | U_{f}U_{f}U_{f}GUCUCUGGUCCUUAC_{f}U_{f}U |
| AS | |
| 40/317502 | U_{f}U_{f}U_{f}GU_{f}C_{f}U_{f}CUGGUCC_{f}U_{f}U_{f}AC_{f}U_{f}U |
| AS | |

Cells were treated with the indicated concentrations of single or double stranded oligomeric compounds shown above using methods described herein. Expression levels of PTEN were determined using real-time PCR methods as described herein, and were compared to levels determined for untreated controls.

% untreated control mRNA

| Construct | 100 nM asRNA | 100 nM siRNA |
|---|---|---|
| 303912 | 35 | 18 |
| 317466 | — | 28 |
| 317408 | — | 18 |
| 317502 | — | 21 |
| 334254 | — | 33 |
| 333756 | 42 | 19 |
| 334257 | 34 | 23 |
| 334255 | 44 | 21 |
| 333752 | 42 | 18 |
| 334253 | 38 | 15 |
| 333750 | 43 | 21 |
| 333749 | 34 | 21 |

Additional siRNAs having 2′-F modified nucleosides are listed below.

| | |
|---|---|
| 37/279471 AS | [symbol] _{f}U_{f}G_{f} [symbol] _{f}U_{f}A_{f}G_{f} [symbol] _{f} [symbol] _{f}U_{f} [symbol] _{f}U_{f}G_{f}G_{f}A_{f}U_{f}U_{f}U_{f} |
| | G_{f}dTdT |
| 36/279467 S | [symbol] _{f}A_{f}A_{f}A_{f}U_{f} [symbol] _{f} [symbol] _{f}A_{f}G_{f}A_{f}G_{f}G_{f} [symbol] _{f}U_{f}A_{f}G_{f} [symbol] _{f}A_{f} |
| | G_{f}dTdT |
| 40/319018 AS | U_{f}U_{f}U_{f}G_{f}U_{f}C_{f}U_{f}C_{f}U_{f}G_{f}G_{f}U_{f}C_{f}C_{f}U_{f}U_{f}A_{f}C_{f} |
| | U_{f}U_{f} |
| 39/319019 S | A_{f}A_{f}G_{f}U_{f}A_{f}A_{f}G_{f}G_{f}A_{f}C_{f}C_{f}A_{f}G_{f}A_{f}G_{f}A_{f}C_{f}A_{f} |
| | A_{f}A_{f} |

### Example 27

### Representative siRNAs Prepared with Fully Modified Antisense Strands (2′-F and 2′-OMe)

siRNA constructs targeting PTEN are prepared wherein the following sense and antisense strands are hybridized. The nucleosides are annotated as to chemical modification as per the legend at the beginning of the examples.

| SEQ ID NO/ | |
|---|---|
| ISIS NO | Sequences 5′-3′ |
| 48/283546 | C_{f}U_{f}GₘC_{f}U_{f}AₘGₘC_{f}C_{f}U_{f}C_{f}U_{f}GₘGₘAₘU_{f}U_{f}U_{f}GₘUₘ |
| (as) | dT |
| 40/336240 | UUUGUCUC_{f}U_{f}GGU_{f}C_{f}CUUACₘUₘUₘ |
| (s) | |

### Example 28

### Representative siRNAs Prepared Having 2′-MOE Modified Nucleosides were Assayed for PTEN mRNA Levels Against Untreated Control

siRNA constructs targeting PTEN were prepared wherein the following antisense strands were hybridized to the complementary full phosphodiester sense strand.

The following antisense strands of siRNAs were hybridized to the complementary full phosphodiester sense strand. The nucleosides are annotated as to chemical modification as per the legend at the beginning of the examples. Linkages are phosphorothioate. Cells were treated with the duplexes using methods described herein. Results obtained using 100 nM duplex are presented as a percentage of untreated control PTEN mRNA levels.

| | | |
|---|---|---|
| | | PTEN |
| | | mRNA |
| SEQ ID | | level |
| NO./ | | (% UTC) |
| ISIS NO. | Composition (5′ to 3′) | 100 nm |

| | | |
|---|---|---|
| 49/xxxxx | UUCAUUCCUGGUCUCUGUUU | — |
| (as) | | |
| 49/xxxxx | UₑUₑCₑAUUCCUGGUCUCUGUUU | 50 |
| (as) | | |
| 49/xxxxx | UUCAₑUₑUₑCCUGGUCUCUGUUU | — |
| (as) | | |
| 49/xxxxx | UUCAUUCₑCₑUₑGGUCUCUGUUU | 43 |
| (as) | | |
| 49/xxxxx | UUCAUUCCUGₑGₑUₑCUCUGUUU | 42 |
| (as) | | |
| 49/xxxxx | UUCAUUCCUGGUCₑUₑCₑUGUUU | 47 |
| (as) | | |
| 49/xxxxx | UUCAUUCCUGGUCUCUₑGₑUₑUU | 63 |
| (as) | | |
| 49/xxxxx | UUCAUUCCUGGUCUCUGUₑUₑUₑ | 106 |
| (as) | | |

### Example 29

### 4′-Thio and 2′-OCH₃ Chimeric Oligomeric Compounds

The double-stranded constructs shown below were prepared (antisense strand followed by the sense strand of the duplex). The “P” following the designation for antisense (as) indicates that the target is PTEN and the “S” indicates that the target is Survivin. The nucleosides are annotated as to chemical modification as per the legend at the beginning of the examples.

| SEQ ID NO./ | |
|---|---|
| ISIS NO. | Composition (5′ to 3′) |
| 40/308743 | UₘUₘUₘGUCUCUGGUCCUUACₘUₘUₘ |
| (as-P) | |
| 39/308746 | AAGUAAGGACCAGAGACAAA |
| (s) | |
| 24/353537 | UₜUₜUₜGAAAAUGUUGAUCUₜCₜCₜ |
| (as-S) | |
| 25/343868 | GGAGAUCAACAUUUUCAAA |
| (s-S) | |
| 24/353537 | UₜUₜUₜGAAAAUGUUGAUCUₜCₜCₜ |
| (as-S) | |
| 25/352512 | GₘGₘAₘGₘAₘUₘCₘAₘAₘCₘAₘUₘUₘUₘUₘCₘAₘAₘAₘ |
| (s) | |
| 24/353537 | UₜUₜUₜGAAAAUGUUGAUCUₜCₜCₜ |
| (as-S) | |
| 25/352513 | GGₘAₘGₘAₘUₘCₘAₘAₘCₘAₘUₘUₘUₘUₘCₘAₘAₘA |
| (s) | |
| 24/353537 | UₜUₜUₜGAAAAUGUUGAUCUₜCₜCₜ |
| (as-S) | |
| 25/352514 | GGₑAGₑAUₑCAₑACₑAUₑUUₑUCₑAAₑA |
| (s) | |

The constructs designed to the targets indicated were tested in accordance with the assays described herein. The duplexed oligomeric compounds were evaluated in HeLa cells (American Type Culture Collection, Manassas Va.). Culture methods used for HeLa cells are available from the ATCC and may be found, for example, at http://www.atcc.org. For cells grown in 96-well plates, wells were washed once with 200 μL OPTI-MEM-1 reduced-serum medium and then treated with 130 μL of OPTI-MEM-1 containing 12 μg/mL LIPOFECTIN™ (Invitrogen Life Technologies, Carlsbad, Calif.) and the dsRNA at the desired concentration. After about 5 hours of treatment, the medium was replaced with fresh medium. Cells were harvested 16 hours after dsRNA treatment, at which time RNA was isolated and target reduction measured by quantitative real-time PCR as described in previous examples. Resulting dose-response data was used to determine the IC50 for each construct.

| Construct | Assay/Species | Target | IC50 (nM) |
|---|---|---|---|
| 308743:308746 | Dose Response/Human PTEN | 0.0275 | |
| 353537:343868 | Dose Response/Human Survivin | 0.067284 | |
| 353537:343868 | Dose Response/Human Survivin | 0.17776 | |
| 353537:343868 | Dose Response/Human Survivin | 0.598 | |
| 353537:343868 | Dose Response/Human Survivin | 4.23 | |
| 353537:352512 | Dose Response/Human Survivin | 0.60192 | |
| 353537:352513 | Dose Response/Human Survivin | 0.71193 | |
| 353537:352514 | Dose Response/Human Survivin | 0.48819 | |

### Example 30

### Selected siRNA Constructs Prepared and Tested Against eIF4E and Survivin Targets

Selected siRNA constructs were prepared and tested for their ability to lower targeted RNA as measured by quantitative real-time PCR. The duplexes are shown below (antisense strand followed by the sense strand of the duplex). The nucleosides are annotated as to chemical modification as per the legend at the beginning of the examples.

| SEQ ID | |
|---|---|
| NO./ | Compostion (5′ to 3′) |
| ISIS NO. | Targeted to eIF4E |
| 50/349894 | U_{f}G_{f}U_{f}C_{f}A_{f}UAUUCCUGGAUₘCₘCₘUₘUₘ |
| (as) | |
| 51/338935 | AAGGAUCCAGGAAUAUGACA |
| (s) | |
| 52/349895 | U_{f}C_{f}C_{f}U_{f}G_{f}GAUCCUUCACCₘAₘAₘUₘGₘ |
| (as) | |
| 53/338939 | CAUUGGUGAAGGAUCCAGGA |
| (s) | |
| 54/349896 | U_{f}C_{f}U_{f}U_{f}A_{f}UCACCUUUAGCₘUₘCₘUₘAₘ |
| (as) | |
| 55/338943 | UAGAGCUAAAGGUGAUAAGA |
| (s) | |
| 56/349897 | A_{f}U_{f}A_{f}C_{f}U_{f}CAGAAGGUGUCₘUₘUₘCₘUₘ |
| (as) | |
| 57/338952 | AGAAGACACCUUCUGAGUAU |
| (s) | |
| 58/352827 | UₛCₛUₛUAUCACCUUUAGCUₘCₘUₘ |
| (as) | |
| 59/342764 | AGAGCUAAAGGUGAUAAGA |
| (s) | |
| 58/354604 | UₛCₛUₛU_{f}A_{f}U_{f}C_{f}A_{f}C_{f}C_{f}U_{f}U_{f}U_{f}A_{f}G_{f}C_{f}UₘCₘUₘ |
| (as) | |
| 59/342764 | AGAGCUAAAGGUGAUAAGA |
| (s) | |
| SEQ ID | |
| NO./ | Compostion (5′ to 3′) |
| ISIS NO. | Targeted to Survivin |
| 24/355710 | U_{f}U_{f}U_{f}G_{f}A_{f}AAAUGUUGAUₘCₘUₘCₘCₘ |
| (as) | |
| 25/343868 | GGAGAUCAACAUUUUCAAA |
| (s) | |
| 24/353540 | UₛUₛUₛGAAAAUGUUGAUCUₘCₘCₘ |
| (as) | |
| 45/343868 | GGAGAUCAACAUUUUCAAA |
| (s) | |

The above constructs were tested in HeLa cells, MH-S cells or U-87 MG cells using transfection procedures and real-time PCR as described herein. The resulting IC₅₀'s for the duplexes were calculated and are shown below.

| Construct | Species/cell line | Gene | IC₅₀ |
|---|---|---|---|
| 349894:338935 | Human/HeLa | eIF4E | 0.165 |
| 349895:338939 | Human/HeLa | eIF4E | 0.655 |
| 349896:338943 | Human/HeLa | eIF4E | 0.277 |
| 349896:338943 | Mouse/MH-S | eIF4E | 0.05771 |
| 349897:338952 | Human/HeLa | eIF4E | 0.471 |
| 352827:342764 | Human/HeLa | eIF4E | 2.033 |
| 352827:342764 | Mouse/MH-S | eIF4E | 0.34081 |
| 354604:342764 | Human/HeLa | eIF4E | 2.5765 |
| 355710:343868 | Human/HeLa | Survivin | 0.048717 |
| 353540:343868 | Human/HeLa | Survivin | 0.11276 |
| 353540:343868 | Human/U-87 MG | Survivin | 0.0921 |

### Example 31

### Positionally Modified Compositions

The table below shows exemplary positionally modified compositions prepared in accordance with the present invention. Target descriptors are: P=PTEN; S=Survivin; E=eIF4E and are indicated following the antisense strand designation.

| SEQ ID NO./ | |
|---|---|
| ISIS NO. | Composition (5′ to 3′) |
| 52/345838 (as-P) | UCCUGGₘAUCCUUₘCACₘCAAₘUₘGₘ |
| 53/338939 (s) | CAUUGGUGAAGGAUCCAGGA |
| 60/345839 (as-E) | CCUGGₘAₘUCCₘUₘUGACCAAₘUₘGₘ |
| 53/338939 (s) | CAUUGGUGAAGGAUCCAGGA |
| 56/345853 (as-E) | AUACUCₘAₘGAAₘGₘGUGUCUUₘCₘUₘ |
| 57/338952 (s) | AGAAGACACCUUCUGAGUAU |
| 24/352505 (as-S) | UUUGAₘAAAₘUGUₘUGAₘUCUₘCₘCₘ |
| 25/343868 (s) | GGAGAUCAACAUUUUCAAA |
| 24/352506 (as-S) | UUUGAAₘAₘAUGₘUₘUGAUCUₘCₘCₘ |
| 25/343868 (s) | GGAGAUCAACAUUUUCAAA |
| 24/352506 (as-S) | UUUGAAₘAₘAUGₘUₘUGAUCUₘCₘCₘ |
| 25/346287 (s) | GGAGAUCAACAUUUUCAAA |
| 24/352505 (as-S) | UUUGAₘAAAₘUGUₘUGAₘUCUₘCₘCₘ |
| 25/346287 (s) | GGAGAUCAACAUUUUCAAA |
| 24/352505 (as-S) | UUUGAₘAAAₘUGUₘUGAₘUCUₘCₘCₘ |
| 25/352511 (s) | GGₘAGₘAUₘCAₘACₘAUₘUUₘUCₘAAₘA |
| 24/352505 (as-S) | UUUGAₘAAAₘUGUₘUGAₘUCUₘCₘCₘ |
| 25/352513 (s) | GGₘAₘGₘAₘUₘCₘAₘAₘCₘAₘUₘUₘUₘUₘCₘAₘ |
| | AₘA |
| 24/352506 (as-S) | UUUGAAₘAₘAUGₘUₘUGAUCUₘCₘCₘ |
| 25/352511 (s) | GGₘAGₘAUₘCAₘACₘAUₘUUₘUCₘAAₘA |
| 24/352505 (as-S) | UUUGAₘAAAₘUGUₘUGAₘUCUₘCₘCₘ |
| 25/352514 (s) | GGₑAGₑAUₑCAₑACₑAUₑUUₑUCₑAAₑA |
| 24/352506 (as-S) | UUUGAAₘAₘAUGₘUₘUGAUCUₘCₘCₘ |
| 25/352514 (s) | GGₑAGₑAUₑCAₑACₑAUₑUUₑUCₑAAₑA |
| 24/352505 (as-S) | UUUGAₘAAAₘUGUₘUGAₘUCUₘCₘCₘ |
| 25/352512 (s) | GₘGₘAₘGₘAₘUₘCₘAₘAₘCₘAₘUₘUₘUₘUₘCₘAₘ |
| | AₘAₘ |
| 56/345853 (as-_{E)} | AUACUCₘAₘGAAₘGₘGUGUCUUₘCₘUₘ |
| 57/345857 (s) | AGₘAₘAₘGₘAₘCₘAₘCₘCₘUₘUₘCₘUₘGₘAₘGₘ |
| | UₘAₘU |
| 24/352506 (as-S) | UUUGAAₘAₘAUGₘUₘUGAUCUₘCₘCₘ |
| 25/352512 (s) | GₘGₘAₘGₘAₘUₘCₘAₘAₘCₘAₘUₘUₘUₘUₘCₘAₘ |
| | AₘAₘ |
| 24/352506 (as-S) | UUUGAAₘAₘAUGₘUₘUGAUCUₘCₘCₘ |
| 25/352513 (s) | GGₘAₘGₘAₘUₘCₘAₘAₘCₘAₘUₘUₘUₘUₘCₘAₘ |
| | AₘA |
| 40/335225 (as-P) | UUUGUCUCUₘGₘGₘUCCUUACUU |
| 39/308746 (s) | AAGUAAGGACCAGAGACAAA |
| 40/335226 (as-P) | UUUGUCₘUₘCₘUGGUCCUUACUU |
| 39/308746 (s) | AAGUAAGGACCAGAGACAAA |
| 40/345711 (as-P) | UUUGₗUCUCUGₗGUCCUUACUₗU |
| 39/308746 (s) | AAGUAAGGACCAGAGACAAA |
| 40/345712 (as-P) | UUUₗGₗUCUCUGₗGₗUCCUUAₗCₗUU |
| 39/308746 (s) | AAGUAAGGACCAGAGACAAA |
| 40/347348 (as-P) | UₗUₗUₗGUCₗUCUₗGGUₗCCUₗUACₗUₗUₗ |
| 39/308746 (s) | AAGUAAGGACCAGAGACAAA |
| 40/348467 (as-P) | UUₗUₗGUCₗUCUₗGGUₗCCUₗUACₗUₗUₗ |
| 39/308746 (s) | AAGUAAGGACCAGAGACAAA |
| 24/355715 (as-S) | UUUGₗAAAAUₗGUUGAUCUCₗC |
| 25/343868 (s) | GGAGAUCAACAUUUUCAAA |
| 40/331426 (as-P) | UUUGUCUCUₗGₗGₗUCCUUACUU |
| 39/308746 (s) | AAGUAAGGACCAGAGACAAA |
| 40/331695 (as-P) | UUUGUCUCUGGUCCUUACₗUₗUₗ |
| 39/308746 (s) | AAGUAAGGACCAGAGACAAA |
| 40/332231 (as-P) | UUUGUCUCUGGUCCUUACUₗU |
| 39/308746 (s) | AAGUAAGGACCAGAGACAAA |
| 24/355712 (as-S) | UUUGAₗAAAₗUGUₗUGAₗUCUₘCₘCₘ |
| 25/343868 (s) | GGAGAUCAACAUUUUCAAA |
| 24/353538 (as-S) | UUUₜGAAAAUₜGUUₜGAUCUₜCₜCₛ |
| 25/343868 (s) | GGAGAUCAACAUUUUCAAA |
| 40/336671 (as-P) | UUUGUCUCUGGUCCUUACₜUₜUₛ |
| 39/308746 (s) | AAGUAAGGACCAGAGACAAA |
| 40/336674 (as-P) | UUUGUCUCUGGUCCUUₜACₜUₜUₛ |
| 39/308746 (s) | AAGUAAGGACCAGAGACAAA |
| 40/336675 (as-P) | UUUGUCUCUGGUCCUUACUUₛ |
| 39/308746 (s) | AAGUAAGGACCAGAGACAAA |
| 40/336672 (as-P) | UUUGUCUCUGGUCₜCₜUₜUACUU |
| 39/308746 (s) | AAGUAAGGACCAGAGACAAA |
| 40/336673 (as-P) | UUUGUCUCUGGUₜCₜCₜUUACUU |
| 39/308746 (s) | AAGUAAGGACCAGAGACAAA |
| 40/336676 (as-P) | UUUGUCUₜCₜUₜGGUCCUUACUU |
| 39/308746 (s) | AAGUAAGGACCAGAGACAAA |
| 40/336678 (as-P) | UₜUₜUₜGUCUCUGGUCCUUACUU |
| 39/308746 (s) | AAGUAAGGACCAGAGACAAA |
| 24/352515 (as-S) | UUUGAAAAUGUUGAUₘCₘUₘCₘCₘ |
| 25/343868 (s) | GGAGAUCAACAUUUUCAAA |
| 61/330919 (as-P) | UUTₑGₑTₑCUCUGGUCCUUACUU |
| 39/308746 (s) | AAGUAAGGACCAGAGACAAA |
| 62/330997 (as-P) | TₑTₑTₑGTCUCUGGUCCUUACUU |
| 39/308746 (s) | AAGUAAGGACCAGAGACAAA |
| 40/333749 (as-P) | UUUGUCUCUGGUCCU_{f}U_{f}A_{f}CUU |
| 39/308746 (s) | AAGUAAGGACCAGAGACAAA |
| 40/333750 (as-P) | UUUGUCUCUGGU_{f}C_{f}C_{f}UUACUU |
| 39/308746 (s) | AAGUAAGGACCAGAGACAAA |
| 40/333752 (as-P) | UUUGUC_{f}U_{f}C_{f}UGGUCCUUACUU |
| 39/308746 (s) | AAGUAAGGACCAGAGACAAA |
| 40/333756 (as-P) | UUUGUCUCUGGUCCUUAC_{f}U_{f}U_{f} |
| 39/308746 (s) | AAGUAAGGACCAGAGACAAA |
| 40/334253 (as-P) | UUUGUCUCUfG_{f}G_{f}UCCUUACUU |
| 39/308746 (s) | AAGUAAGGACCAGAGACAAA |
| 24/353539 (as-S) | UₜUₜUₜGAAAAUₜGUUₜGAUCUₘCₘCₘ |
| 25/343868 (s) | GGAGAUCAACAUUUUCAAA |

The above constructs were tested in HeLa cells, MH-S cells or U-87 MG cells using methods described herein. Resulting IC₅₀'s were calculated and are shown below. Also shown are the species to which the compounds were targeted and the cell line in which they were assayed.

| Construct | Species/Cell Line | Gene | IC50 |
|---|---|---|---|
| 345838:338939 | Mouse/MH-S | eIF4E | 0.022859 |
| 345839:338939 | Mouse/MH-S | eIF4E | 0.01205 |
| 345853:338952 | Mouse/MH-S | eIF4E | 0.075517 |
| 352505:343868 | Human/HeLA | Survivin | 0.17024 |
| 352506:343868 | Human/HeLA | Survivin | 0.055386 |
| 352506:346287 | Human/HeLA | Survivin | 0.11222 |
| 352505:346287 | Human/HeLA | Survivin | 0.96445 |
| 352505:352511 | Human/HeLA | Survivin | 0.21527 |
| 352505:352513 | Human/HeLA | Survivin | 0.12453 |
| 352506:352511 | Human/HeLA | Survivin | 0.045167 |
| 352505:352514 | Human/HeLA | Survivin | 0.47593 |
| 352506:352514 | Human/HeLA | Survivin | 0.11759 |
| 352506:352514 | Human/HeLA | Survivin | 0.376 |
| 352506:352514 | Human/U-87 MG | Survivin | 0.261 |
| 352505:352512 | Human/HeLA | Survivin | 0.075608 |
| 345853:345857 | Mouse/MH-S | eIF4E | 0.025677 |
| 352506:352512 | Human/HeLA | Survivin | 0.11093 |
| 352506:352513 | Human/HeLA | Survivin | 0.24503 |
| 335225:308746 | Human/HeLA | PTEN | 0.809 |
| 335226:308746 | Human/HeLA | PTEN | 1.57 |
| 308746:345711 | Human/HeLA | PTEN | 1.13 |
| 308746:345712 | Human/HeLA | PTEN | 0.371 |
| 308746:347348 | Human/HeLA | PTEN | 0.769 |
| 308746:348467 | Human/HeLA | PTEN | 18.4 |
| 355715:343868 | Human/HeLA | Survivin | 0.020825 |
| 331426:308746 | Human/HeLA | PTEN | 0.5627 |
| 331695:308746 | Human/HeLA | PTEN | 0.27688 |
| 332231:308746 | Human/HeLA | PTEN | 5.58 |
| 355712:343868 | Human/HeLA | Survivin | 0.022046 |
| 353538:343868 | Human/HeLA | Survivin | 0.491 |
| 353538:343868 | Human/U87-MG | Survivin | 0.46 |
| 336671:308746 | Human/HeLA | PTEN | 0.273 |
| 336674:308746 | Human/HeLA | PTEN | 0.363 |
| 336675:308746 | Human/HeLA | PTEN | 0.131 |
| 336672:308746 | Human/HeLA | PTEN | 0.428 |
| 336673:308746 | Human/HeLA | PTEN | 0.122 |
| 336676:308746 | Human/HeLA | PTEN | 7.08 |
| 336678:308746 | Human/HeLA | PTEN | 0.144 |
| 352515:343868 | Human/HeLA | Survivin | 0.031541 |
| 330919:308746 | Human/HeLA | PTEN | 29.4 |
| 330997:308746 | Human/HeLA | PTEN | 3.39 |
| 333749:308746 | Human/HeLA | PTEN | 1.3 |
| 333750:308746 | Human/HeLA | PTEN | 0.30815 |
| 333752:308746 | Human/HeLA | PTEN | 1.5416 |
| 333756:308746 | Human/HeLA | PTEN | 1.0933 |
| 334253:308746 | Human/HeLA | PTEN | 0.68552 |
| 353539:343868 | Human/HeLA | Survivin | 0.13216 |

### Example 32

### Suitable Positional Compositions of the Invention

The following table describes some suitable positional compositions of the invention. In the listed constructs, the 5′-terminal nucleoside or the sense (upper) strand is hybridized to the 3′-terminal nucleoside of the antisense (lower) strand.

| Compound | |
|---|---|
| (sense/antisense) | Construct (sense 5′→3′/antisense) |
| sense RNA | 5′-XXXXXXXXXXXXXXXXXXX-3′ |
| 4′thio (bold) dispersed antisense | 3′-XXX₁₇XXXXX₁₂XXX₉XXXXXX₃X₂X₁-5′ |
| Sense RNA | 5′-XXXXXXXXXXXXXXXXXXX-3′ |
| 2′-OMe (italic)/4′-thio (bold) | 3′-X₁₉X₁₈X₁₇XXXXXXXXXXXXXXXX-5′ |
| dispersed antisense | |
| Sense RNA | 5′-XXXXXXXXXXXXXXXXXXXX-3′ |
| Chimeric 2′-OMe (italic)/2′- | 3′-XXX [symbol] XX [symbol] XX [symbol] -5′ |
| fluoro(bold italic) antisense | |
| Alternate MOE(underline)/OH sense | 5′-XXXXXXXXXXXXXXXXXXX-3′ |
| Chimeric OMe (italic)/OH | 3′-X₂₀X₁₉X₁₈XXXXXXX₁₁X₁₀XXX₇X₆XXXXX-5′ |
| antisense | |
| OMe Gapmer Sense/ | 5′-XXXXXXXXXXXXXXXXXXXX-3′ |
| Chimeric OMe (italic)/OH | 3′-X₂₀X₁₉X₁₈XXX₁₅XXX₁₂XXXXXX₆XXXXX-5′ |
| antisense | |
| Sense RNA | 5′-XXXXXXXXXXXXXXXXXXXX-3′ |
| Chimeric OMe/OH antisense. | 3′-XXX₁₇XXX₁₄XXX₁₁XXX₈XXX₅XXXX-5′ |

### Example 33

### Alternating 2′-O-Methyl/2′-F 20mer siRNAs Targeting PTEN in T-24 Cells

A dose response experiment was performed in the PTEN system to examine the positional effects of alternating 2′-O-Methyl/2′-F siRNAs. The nucleosides are annotated as to chemical modification as per the legend at the beginning of the examples.

| SEQ ID | |
|---|---|
| NO./ | |
| ISIS NO. | Composition (5′ to 3′) |
| 40/303912 | UUUGUCUCUGGUCCUUACUU |
| (as) | |
| 39/308746 | P-AAGUAAGGACCAGAGACAAA |
| (s) | |
| 40/340569 | P-U_{f}UₘU_{f}GₘU_{f}CₘU_{f}CₘU_{f}GₘG_{f}UₘC_{f}CₘU_{f}UₘA_{f}CₘU_{f} |
| (as) | Uₘ |
| 39/340573 | P-A_{f}AₘG_{f}UₘA_{f}AₘG_{f}GₘA_{f}CₘC_{f}AₘG_{f}AₘG_{f}AₘC_{f}AₘA_{f} |
| (s) | Aₘ |
| 40/340569 | P-U_{f}UₘU_{f}GₘU_{f}CₘU_{f}CₘU_{f}GₘG_{f}UₘC_{f}CₘU_{f}UₘA_{f}CₘU_{f} |
| (as) | Uₘ |
| 39/340574 | P-AₘA_{f}GₘU_{f}AₘA_{f}GₘG_{f}AₘC_{f}CₘA_{f}GₘA_{f}GₘA_{f}CₘA_{f}Aₘ |
| (s) | A_{f} |
| 40/340569 | P-U_{f}UₘU_{f}GₘU_{f}CₘU_{f}CₘU_{f}GₘG_{f}UₘC_{f}CₘU_{f}UₘA_{f}CₘU_{f} |
| (as) | Uₘ |
| 39/308746 | P-AAGUAAGGACCAGAGACAAA |
| (s) | |
| 40/340570 | P-U_{f}UₘU_{f}GₘU_{f}CₘU_{f}CₘU_{f}GₘG |
| (as) | _{f}UₘC_{f}CₘU_{f}UₘA_{f}CₘU_{f}Uₘ |
| 39/340573 | P-A_{f}AₘG_{f}UₘA_{f}AₘG_{f}GₘA_{f}CₘC_{f}AₘG_{f}AₘG_{f}AₘC_{f}AₘA_{f} |
| (s) | Aₘ |
| 40/340570 | P-U_{f}UₘU_{f}GₘU_{f}CₘU_{f}CₘU_{f}GₘG |
| (as) | _{f}UₘC_{f}CₘU_{f}UₘA_{f}CₘU_{f}Uₘ |
| 39/340574 | P-AₘA_{f}GₘU_{f}AₘA_{f}GₘG_{f}AₘC_{f}CₘA_{f}GₘA_{f}GₘA_{f}CₘA_{f}Aₘ |
| (s) | A_{f} |
| 40/340570 | P-U_{f}UₘU_{f}GₘU_{f}CₘU_{f}CₘU_{f}GₘG |
| (as) | _{f}UₘC_{f}CₘU_{f}UₘA_{f}CₘU_{f}Uₘ |
| 39/308746 | P-AAGUAAGGACCAGAGACAAA |
| (s) | |

The above siRNA constructs were assayed to determine the effects of the full alternating 2′-O-methyl/2′-F antisense strands (PO or PS) where the 5′-terminus of the antisense strands are 2′-F modified nucleosides with the remaining positions alternating. The sense strands were prepared with the positioning of the modified nucleosides in both orientations such that for each siRNA tested with 2′-O-methyl modified nucleosides beginning at the 3′-terminus of the sense strand another identical siRNA was prepared with 2′-F modified nucleosides beginning at the 3′-terminus of the sense strand. Another way to describe the differences between these two siRNAs is that the register of the sense strand is in both possible orientations with the register of the antisense strand being held constant in one orientation. Activity of the constructs (at 150 nM) is presented below as a percentage of untreated control.

| siRNA | Activity (% untreated control 150 nM) | |
|---|---|---|
| Construct | Sense | Antisense |
| 308746/303912 28% | PO unmodified RNA | PS unmodified RNA |
| 340574/340569 46% | PO (2′-F, 3′-0) | PO (2′-F, 5′-0) |
| 340574/340570 62% | PO (2′-F, 3′-0) | PS (2′-F, 5′-0) |
| 340573/340569 84% | PO (2′-O-methyl, 3′-0) | PO (2′-F, 5′-0) |
| 340573/340570 23% | PO (2′-O-methyl, 3′-0) | PS (2′-F, 5′-0) |
| 308746/340569 23% | PO unmodified RNA | PO (2′-F, 5′-0) |
| 308746/340570 38% | PO unmodified RNA | PS (2′-F, 5′-0) |

Within the alternating motif for this assay the antisense strands were prepared beginning with a 2′-F group at the 5′-terminal nucleoside. The sense strands were prepared with the alternating motif beginning at the 3′-terminal nucleoside with either the 2′-F modified nucleoside or a 2′-O-methyl modified nucleoside. The siRNA constructs were prepared with the internucleoside linkages for the sense strand as full phosphodiester and the internucleoside linkages for the antisense strands as either full phosphodiester or phosphorothioate.

### Example 34

### Effect of Modified Phosphate Moieties on Alternating 2′-O-methyl/2′-F siRNAs Targeting eIF4E

A dose response was performed targeting eIF4E in HeLa cells to determine the effects of selected terminal groups on activity. More specifically the reduction of eIF4E mRNA in HeLa cells by 19-basepair siRNA containing alternating 2′-OMe/2′-F modifications is shown in this example. The nucleosides are annotated as to chemical modification as per the legend at the beginning of the examples. 5′-P(S) is a 5′-thiophosphate group (5′-O—P(═S)(OH)OH), 5′-P(H) is a 5′-H-phosphonate group (5′-O—P(═O)(H)OH) and 5′-P(CH₃) is a methylphosphonate group (5′-O—P(═O)(CH₃)OH). All of the constructs in this assay were full phosphodiester linked.

HeLa cells were plated at 4000/well and transfected with siRNA in the presence of LIPOFECTIN™ (6 μL/mL OPTI-MEM) and treated for about 4 hours, re-fed, lysed the following day and analyzed using real-time PCR methods as described herein. The maximum % reduction is the amount of mRNA reduction compared to untreated control cells at the highest concentration (100 nM), with IC50 indicating the interpolated concentration at which 50% reduction is achieved.

SEQ ID NO/

| | |
|---|---|
| ISIS NO | SEQUENCES 5′-3′ targeted to eIF4E |

| | |
|---|---|
| 26/341391 (as) | UUGUCUCUGGUCCUUACUU |
| 27/341401 (s) | AAGUAAGGACCAGAGACAA |
| 58/342744 (as) | UCUUAUCACCUUUAGCUCU |
| 59/342764 (s) | AGAGCUAAAGGUGAUAAGA |
| 58/351831 (as) | UₘC_{f}UₘU_{f}AₘU_{f}CₘA_{f}CₘC_{f}UₘU_{f}UₘA_{f}GₘC_{f}UₘC_{f} |
| | Uₘ |
| 59/351832 (s) | A_{f}GₘA_{f}GₘC_{f}UₘA_{f}AₘA_{f}GₘG_{f}UₘG_{f}AₘU_{f}AₘA_{f}Gₘ |
| | A_{f} |
| 58/368681 (as) | P-UₘC_{f}UₘU_{f}AₘU_{f}CₘA_{f}CₘC_{f}UₘU_{f}UₘA_{f}GₘC_{f}Uₘ |
| | C_{f}Uₘ |
| 59/351832 (s) | A_{f}GₘA_{f}GₘC_{f}UₘA_{f}AₘA_{f}GₘG_{f}UₘG_{f}AₘU_{f}AₘA_{f}Gₘ |
| | A_{f} |
| 58/379225 (as) | P(S)-UₘC_{f}UₘU_{f}AₘU_{f}CₘA_{f}CₘC_{f}UₘU_{f}UₘA_{f}GₘC |
| | _{f}UₘC_{f}Uₘ |
| 59/351832 (s) | A_{f}GₘA_{f}GₘC_{f}UₘA_{f}AₘA_{f}GₘG_{f}UₘG_{f}AₘU_{f}AₘA_{f}Gₘ |
| | A_{f} |
| 58/379712 (as) | P(H)-UₘC_{f}UₘU_{f}AₘU_{f}CₘA_{f}CₘC_{f}UₘU_{f}UₘA_{f}GₘC |
| | _{f}UₘC_{f}Uₘ |
| 59/351832 (s) | A_{f}GₘA_{f}GₘC_{f}UₘA_{f}AₘA_{f}GₘG_{f}UₘG_{f}AₘU_{f}AₘA_{f}Gₘ |
| | A_{f} |
| 58/379226 (as) | P(CH₃)-UₘC_{f}UₘU_{f}AₘU_{f}CₘA_{f}CₘC_{f}UₘU_{f}UₘA_{f}G |
| | ₘC_{f}UₘC_{f}Uₘ |
| 59/351832 (s) | A_{f}GₘA_{f}GₘC_{f}UₘA_{f}AₘA_{f}GₘG_{f}UₘG_{f}AₘU_{f}AₘA_{f}Gₘ |
| | A_{f} |

| Double stranded construct | | Activity | |
|---|---|---|---|
| Antisense | Sense | % Control (100 nM) | IC50 (nM) |
| 341401 | 341391 | 103 | n/a (neg control) |
| 342764 | 342744 | 11.0 | 1.26 |
| 351832 | 351831 | 3.5 | 0.66 |
| 351832 | 368681 | 3.6 | 0.14 |
| 351832 | 379225 | 2.8 | 0.20 |
| 351832 | 379712 | 8.0 | 2.01 |
| 351832 | 379226 | 18.1 | 8.24 |

### Example 35

### Assay of Selected siRNAs Targeting PTEN

The constructs listed below were assayed for activity by measuring the levels of human PTEN mRNA in HeLa cells against untreated control levels. The nucleosides are annotated as to chemical modification as per the legend at the beginning of the examples. “P(S)—” indicates a thiophosphate group (—O—P(═S)(OH)OH).

SEQ ID NO/

| | |
|---|---|
| ISIS NO | SEQUENCES 5′-3′ targeted to PTEN |

| | |
|---|---|
| 26/371789 (as) | P-UUGUCUCUGGUCCUUACUU |
| 27/341401 (s) | P-AAGUAAGGACCAGAGACAA |
| 26/383498 (as) | UₘU_{f}GₘU_{f}CₘU_{f}CₘU_{f}GₘG_{f}UₘC_{f}CₘU_{f}UₘA_{f}CₘU_{f} |
| | Uₘ |
| 27/359351 (s) | AₑAₑGₑUAAGGACCAGAGACₑAₑAₑ |
| 26/381671 (as) | P-UₘU_{f}GₘU_{f}CₘU_{f}CₘU_{f}GₘG_{f}UₘC_{f}CₘU_{f}UₘA_{f}Cₘ |
| | U_{f}Uₘ |
| 27/359351 (s) | AₑAₑGₑUAAGGACCAGAGACₑAₑAₑ |
| 26/382716 (as) | P(S)-UₘU_{f}GₘU_{f}CₘU_{f}CₘU_{f}GₘG_{f}UₘC_{f}CₘU_{f}UₘA |
| | _{f}CₘU_{f}Uₘ |
| 27/359351 (s) | AₑAₑGₑUAAGGACCAGAGACₑAₑAₑ |
| 26/381672 (as) | P-UₘU_{f}GₘU_{f}CₘU_{f}CₘU_{f}GₘG_{f}UₘC_{f}CₘU_{f}UₘA_{f}Cₘ |
| | U_{f}Uₘ |
| 27/359351 (s) | AₑAₑGₑUAAGGACCAGAGACₑAₑAₑ |
| 26/384758 (as) | P(S)-UₜUₜGUCUₘCₘUGGₘUₘCCUUACₘUₘUₘ |
| 27/359351 (s) | AₑAₑGₑUAAGGACCAGAGACₑAₑAₑ |
| 26/384759 (as) | P(S)-UₜUₜGUCUₘCₘUGGₘUₘCCUUACₘUₘUₘ |
| 27/359351 (s) | AₑAₑGₑUAAGGACCAGAGACₑAₑAₑ |
| 26/384760 (as) | P(S)-UₜUₜGUCUCUGGₘUₘCCUUACₘUₘUₘ |
| 27/359351 (s) | AₑAₑGₑUAAGGACCAGAGACₑAₑAₑ |
| 26/384761 (as) | P(S)-UₜUₜGUCUCUGGₘUₘCCUUACₘUₘUₘ |
| 27/359351 (s) | AₑAₑGₑUAAGGACCAGAGACₑAₑAₑ |
| 26/359455 (as) | UUGUCUCUGGUCCUUACUU |
| 27/359351 (s) | AₑAₑGₑUAAGGACCAGAGACₑAₑAₑ |
| 26/384754 (as) | P(S)-UUGUCUₘCₘUGGₘUₘCCUUACₘUₘUₘ |
| 27/359351 (s) | AₑAₑGₑUAAGGACCAGAGACₑAₑAₑ |
| 26/384755 (as) | P(S)-UₜUₜGUCUCUGGUCCUUACₘUₘUₘ |
| 27/359351 (s) | AₑAₑGₑUAAGGACCAGAGACₑAₑAₑ |
| 26/384756 (as) | P(S)-UₜUₜGUCUCUGGUCCUUACₘUₘUₘ |
| 27/359351 (s) | AₑAₑGₑUAAGGACCAGAGACₑAₑAₑ |
| 26/384757 (as) | UₜUₜGUCUₘCₘUGGₘUₘCCUUACₘUₘUₘ |
| 27/359351 (s) | AₑAₑGₑUAAGGACCAGAGACₑAₑAₑ |
| 26/359455 (as) | UUGUCUCUGGUCCUUACUU |
| 27/384762 (s) | AₑAₑGₑUAAGGACCAGAGACₜAₜAₜ |
| 26/384754 (as) | P(S)-UUGUCUₘCₘUGGₘUₘCCUUACₘUₘUₘ |
| 27/384762 (s) | AₑAₑGₑUAAGGACCAGAGACₜAₜAₜ |
| 26/384755 (as) | P(S)-UₜUₜGUCUCUGGUCCUUACₘUₘUₘ |
| 27/384762 (s) | AₑAₑGₑUAAGGACCAGAGACₜAₜAₜ |
| 26/384756 (as) | P(S)-UₜUₜGUCUCUGGUCCUUACₘUₘUₘ |
| 27/384762 (s) | AₑAₑGₑUAAGGACCAGAGACₜAₜAₜ |
| 26/384757 (as) | UₜUₜGUCUₘCₘUGGₘUₘCCUUACₘUₘUₘ |
| 27/384762 (s) | AₑAₑGₑUAAGGACCAGAGACₜAₜAₜ |
| 26/383498 (as) | UₘU_{f}GₘU_{f}CₘU_{f}CₘU_{f}GₘG_{f}UₘC_{f}CₘU_{f}UₘA_{f}CₘU_{f} |
| | Uₘ |
| 27/384762 (s) | AₑAₑGₑUAAGGACCAGAGACₜAₜAₜ |
| 26/381671 (as) | P-UₘU_{f}GₘU_{f}CₘU_{f}CₘU_{f}GₘG_{f}UₘC_{f}CₘU_{f}UₘA_{f}Cₘ |
| | U_{f}Uₘ |
| 27/384762 (s) | AₑAₑGₑUAAGGACCAGAGACₜAₜAₜ |
| 26/382716 (as) | P(S)-UₘU_{f}GₘU_{f}CₘU_{f}CₘU_{f}GₘG_{f}UₘC_{f}CₘU_{f}UₘA |
| | _{f}CₘU_{f}Uₘ |
| 27/384762 (s) | AₑAₑGₑUAAGGACCAGAGACₜAₜAₜ |
| 26/381672 (as) | P-UₘU_{f}GₘU_{f}CₘU_{f}CₘU_{f}GₘG_{f}UₘC_{f}CₘU_{f}UₘA_{f}Cₘ |
| | U_{f}Uₘ |
| 27/384762 (s) | AₑAₑGₑUAAGGACCAGAGACₜAₜAₜ |
| 26/384758 (as) | P(S)-UₜUₜGUCUₘCₘUGGₘUₘCCUUACₘUₘUₘ |
| 27/384762 (s) | AₑAₑGₑUAAGGACCAGAGACₜAₜAₜ |
| 26/384759 (as) | P(S)-UₜUₜGUCUₘCₘUGGₘUₘCCUUACₘUₘUₘ |
| 27/384762 (s) | AₑAₑGₑUAAGGACCAGAGACₜAₜAₜ |
| 26/384760 (as) | P(S)-UₜUₜGUCUCUGGₘUₘCCUUACₘUₘUₘ |
| 27/384762 (s) | AₑAₑGₑUAAGGACCAGAGACₜAₜAₜ |
| 26/384761 (as) | P(S)-UₜUₜGUCUCUGGₘUₘCCUUACₘUₘUₘ |
| 27/384762 (s) | AₑAₑGₑUAAGGACCAGAGACₜAₜAₜ |
| 26/384758 (as) | P(S)-UₜUₜGUCUₘCₘUGGₘUₘCCUUACₘUₘUₘ |
| 27/366023 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/384759 (as) | P(S)-UₜUₜGUCUₘCₘUGGₘUₘCCUUACₘUₘUₘ |
| 27/366023 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/384760 (as) | P(S)-UₜUₜGUCUCUGGₘUₘCCUUACₘUₘUₘ |
| 27/366023 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/384761 (as) | P(S)-UₜUₜGUCUCUGGₘUₘCCUUACₘUₘUₘ |
| 27/366023 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/384754 (as) | P(S)-UUGUCUₘCₘUGGₘUₘCCUUACₘUₘUₘ |
| 27/359351 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/384755 (as) | P(S)-UₜUₜGUCUCUGGUCCUUACₘUₘUₘ |
| 27/359351 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/384756 (as) | P(S)-UₜUₜGUCUCUGGUCCUUACₘUₘUₘ |
| 27/359351 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/384757 (as) | UₜUₜGUCUₘCₘUGGₘUₘCCUUACₘUₘUₘ |
| 27/359351 (s) | A_{f}AₘG_{f}UₘA_{f}AₘG_{f}GₘA_{f}CₘC_{f}AₘG_{f}AₘG_{f}AₘC_{f}Aₘ |
| | A_{f} |
| 26/359345 (as) | UₜUₜGUCUCUGGUCCUUACUₜUₜ |
| 27/384762 (s) | AₑAₑGₑUAAGGACCAGAGACₜAₜAₜ |
| 26/381671 (as) | UₜUₜGUCUCUGGUCCUUACₘUₘUₘ |
| 27/384762 (s) | AₑAₑGₑUAAGGACCAGAGACₜAₜAₜ |
| 26/352820 (as) | P-UₘU_{f}GₘU_{f}CₘU_{f}CₘU_{f}GₘG_{f}UₘC_{f}CₘU_{f}UₘA_{f}Cₘ |
| | U_{f}Uₘ |
| 27/384762 (s) | AₑAₑGₑUAAGGACCAGAGACₜAₜAₜ |
| 26/352820 (as) | P-UₘU_{f}GₘU_{f}CₘU_{f}CₘU_{f}GₘG_{f}UₘC_{f}CₘU_{f}UₘA_{f}Cₘ |
| | U_{f}Uₘ |
| 27/359351 (s) | AₑAₑGₑUAAGGACCAGAGACₑAₑAₑ |
| 26/384754 (as) | P(S)-UUGUCUₘCₘUGGₘUₘCCUUACₘUₘUₘ |
| 27/359351 (s) | A_{f}AₘG_{f}UₘA_{f}AₘG_{f}GₘA_{f}CₘC_{f}AₘG_{f}AₘG_{f}AₘC_{f}Aₘ |
| | A_{f} |

| | |
|---|---|
| Double stranded construct | Activity |

| Antisense | Sense | IC50 (nM) |
|---|---|---|
| 341391 | 341401 | 0.152 |
| 359980 | 359351 | 0.042 |
| 384758 | 359351 | 0.095 |
| 384759 | 359351 | 0.08 |
| 384760 | 359351 | 0.133 |
| 384761 | 359351 | 0.13 |
| 384754 | 359351 | 0.203 |
| 384757 | 359351 | 0.073 |
| 352820 | 359351 | 0.214 |
| 359980 | 384762 | 0.16 |
| 384754 | 384762 | 0.245 |
| 384755 | 384762 | 0.484 |
| 384756 | 384762 | 0.577 |
| 384757 | 384762 | 0.131 |
| 384758 | 384762 | 0.361 |
| 384759 | 384762 | 0.332 |
| 384760 | 384762 | 0.566 |
| 384761 | 384762 | 0.362 |
| 359345 | 384762 | 0.155 |
| 359346 | 384762 | 0.355 |
| 352820 | 384762 | 0.474 |

### Example 36

### Alternating 2′-MOE/2′-OH siRNAs Targeting PTEN

The constructs listed below targeting PTEN were duplexed as shown (antisense strand followed by the sense strand of the duplex) and assayed for activity using methods described herein. The nucleosides are annotated as to chemical modification as per the legend at the beginning of the examples.

| | | |
|---|---|---|
| SEQ ID NO/ | | IC50 |
| ISIS NO | SEQUENCES 5′-3′ targeted to PTEN | (nM) |

| | | |
|---|---|---|
| 27/355771 (s) | P-AAₑGUₑAAₑGGₑACₑCAₑGAₑGAₑCAₑA | 273 |
| 40/357276 | P-UUUGₑUCUCₑUGGUCCUUₑACUU | |
| (as) | | |
| 27/355771 (s) | P-AAₑGUₑAAₑGGₑACₑCAₑGAₑGAₑCAₑA | 5.5 |
| 40/357276 | P-UUUGₑUCUCUGGₑUCCUUACUₑU | |
| (as) | | |

### Example 37

### Chemically Modified siRNA Targeted to PTEN: In Vivo Study

Six- to seven-week old Balb/c mice (Jackson Laboratory, Bar Harbor, Me.) were injected with single strand and double strand compositions targeted to PTEN. The nucleosides are annotated as to chemical modification as per the legend at the beginning of the examples. Each treatment group was comprised of four animals. Animals were dosed via intraperitoneal injection twice per day for 4.5 days, for a total of 9 doses per animal. Saline-injected animals served as negative controls. Animals were sacrificed 6 hours after the last dose was administered, and plasma samples and tissues were harvested. Target reduction in liver was also measured at the conclusion of the study.

SEQ ID NO/

| | |
|---|---|
| ISIS NO | SEQUENCES 5′-3′ targeted to eIF4E |

| | |
|---|---|
| 63/116847 | CₑTₑGₑCₑTₑAGCCTCTGGATₑTₑTₑGₑAₑ |
| | single strand |
| 26/341391 (as) | UUGUCUCUGGUCCUUACUU |
| 27/341401 (s) | AAGUAAGGACCAGAGACAA |
| 26/359995 (as) | UₘU_{f}GₘU_{f}CₘU_{f}CₘU_{f}GₘG_{f}UₘC_{f}CₘU_{f}UₘA_{f}CₘU_{f} |
| | Uₘ |
| 27/359996 (s) | A_{f}AₘG_{f}UₘA_{f}AₘG_{f}GₘA_{f}CₘC_{f}AₘG_{f}AₘG_{f}AₘC_{f}Aₘ |
| | A_{f} |

Two different doses of each treatment were tested. Treatment with ISIS 116847, was administered at doses of 12.5 mg/kg twice daily or at 6.25 mg/kg twice daily.

The siRNA constructs described above (unmodified 341391/341401, 359995/359996 both strands modified) were administered at doses of 25 mg/kg twice daily or 6.25 mg/kg twice daily. Each siRNA is composed of an antisense strand and a complementary sense strand as per previous examples, with the antisense strand targeted to mouse PTEN. ISIS 116847 and all of the siRNAs of this experiment also have perfect complementarity with human PTEN.

PTEN mRNA levels in liver were measured at the end of the study using real-time PCR and RIBOGREEN™ RNA quantification reagent (Molecular Probes, Inc. Eugene, Oreg.) as taught in previous examples above. Results are presented in the table below as the average % inhibition of mRNA expression for each treatment group, normalized to saline-injected control.

Target reduction by modified siRNAs
targeted to PTEN in mouse liver

| | |
|---|---|
| Dose (mg/kg, | |
| administered | % Inhibition |

| Treatment | 2×/day) | Ribogreen | GAPDH |
|---|---|---|---|
| ISIS 116847 | 12.5 | 92 | 95 |
| | 6.25 | 92 | 95 |
| ISIS 341391/341401 | 25 | 12 | 21 |
| | 6.25 | 2 | 9 |
| ISIS 359995/359996 | 25 | 6 | 13 |
| | 6.25 | 5 | 13 |

As shown in the Table above, all oligonucleotides targeted to PTEN caused a reduction in mRNA levels in liver as compared to saline-treated control. The mRNA levels measured for the ISIS 341391/341401 duplex are also suggestive of dose-dependent inhibition.

The effects of treatment with the RNA duplexes on plasma glucose levels were evaluated in the mice treated as described above. Glucose levels were measured using routine clinical analyzer instruments (eg. Ascencia Glucometer Elite XL, Bayer, Tarrytown, N.Y.). Approximate average plasma glucose is presented in the Table below for each treatment group.

Effects of modified siRNAs targeted to PTEN
on plasma glucose levels in normal mice

| | Dose (mg/kg, | |
|---|---|---|
| | administered | Plasma glucose |
| Treatment | 2×/day) | (mg/dL) |
| Saline | N/A | 186 |
| ISIS 116847 | 12.5 | 169 |
| | 6.25 | 166 |
| ISIS 341391/341401 | 25 | 159 |
| | 6.25 | 182 |
| ISIS 359996/359995 | 25 | 182 |
| | 6.25 | 169 |

To assess the physiological effects resulting from in vivo siRNA targeted to PTEN mRNA, the mice were evaluated at the end of the treatment period for plasma triglycerides, plasma cholesterol, and plasma transaminase levels. Routine clinical analyzer instruments (eg. Olympus Clinical Analyzer, Melville, N.Y.) were used to measure plasma triglycerides, cholesterol, and transaminase levels. Plasma cholesterol levels from animals treated with either dose of ISIS 116847 were increased about 20% over levels measured for saline-treated animals. Conversely, the cholesterol levels measured for animals treated with either the 25 mg/kg or the 6.25 mg/kg doses of the ISIS 341391/341401 duplex were decreased about 12% as compared to saline-treated controls. The ISIS 359996/359995 duplex did not cause significant alterations in cholesterol levels. All of the treatment groups showed decreased plasma triglycerides as compared to saline-treated control, regardless of treatment dose.

Increases in the transaminases ALT and AST can indicate hepatotoxicity. The transaminase levels measured for mice treated with the siRNA duplexes were not elevated to a level indicative of hepatotoxicity with respect to saline treated control. Treatment with 12.5 mg/kg doses of ISIS 116847 caused approximately 7-fold and 3-fold increases in ALT and AST levels, respectively. Treatment with the lower doses (6.25 mg/kg) of ISIS 116847 caused approximately 4-fold and 2-fold increases in ALT and AST levels, respectively.

At the end of the study, liver, white adipose tissue (WAT), spleen, and kidney were harvested from animals treated with the oligomeric compounds and were weighed to assess gross organ alterations. Approximate average tissue weights for each treatment group are presented in the table below.

Effects of chemically modified siRNAs targeted
to PTEN on tissue weight in normal mice

| | Dose (mg/kg, | | | | |
|---|---|---|---|---|---|
| | administered | Liver | WAT | Spleen | Kidney |
| Treatment | 2×/day) | Tissue weight (g) | | | |
| Saline | N/A | 1.0 | 0.5 | 0.1 | 0.3 |
| ISIS 116847 | 12.5 | 1.1 | 0.4 | 0.1 | 0.3 |
| | 6.25 | 1.1 | 0.4 | 0.1 | 0.3 |
| ISIS 341391/341401 | 25 | 1.0 | 0.3 | 0.1 | 0.3 |
| | 6.25 | 0.9 | 0.4 | 0.1 | 0.3 |
| ISIS 359996/359995 | 25 | 1.1 | 0.4 | 0.1 | 0.3 |
| | 6.25 | 1.0 | 0.3 | 0.1 | 0.4 |

As shown, treatment with antisense oligonucleotides or siRNA duplexes targeted to PTEN did not substantially alter liver, WAT, spleen, or kidney weights in normal mice as compared to the organ weights of mice treated with saline alone.

### Example 38

### Chemically Modified siRNA Targeted to PTEN: In Vivo Study

Six- to seven-week old Balb/c mice (Jackson Laboratory, Bar Harbor, Me.) were injected with compounds targeted to PTEN. Each treatment group was comprised of four animals. Animals were dosed via intraperitoneal injection twice per day for 4.5 days, for a total of 9 doses per animal. Saline-injected animals served as negative controls. Animals were sacrificed 6 hours after the last dose of oligonucleotide was administered, and plasma samples and tissues were harvested. Target reduction in liver was also measured at the conclusion of the study.

Two doses of each treatment were tested. Treatment with ISIS 116847 (5′-CTGCTAGCCTCTGGATTTGA-3′, SEQ ID NO: 63), a 5-10-5 gapmer was administered at doses of 12.5 mg/kg twice daily or at 6.25 mg/kg twice daily. The siRNA compounds described below were administered at doses of 25 mg/kg twice daily or 6.25 mg/kg twice daily. Each siRNA is composed of an antisense and complement strand as described in previous examples, with the antisense strand targeted to mouse PTEN. ISIS 116847 and all of the siRNAs of this experiment also have perfect complementarity with human PTEN.

An siRNA duplex targeted to PTEN is comprised of antisense strand ISIS 341391 (5′-UUGUCUCUGGUCCUUACUU-3′, SEQ ID NO: 26) and the sense strand ISIS 341401 (5′-AAGUAAGGACCAGAGACAA-3′, SEQ ID NO: 27). Both strands of the ISIS 341391/341401 duplex are comprised of ribonucleosides with phosphodiester internucleoside linkages.

Another siRNA duplex targeted to human PTEN is comprised of antisense strand ISIS 342851 (5′-UUUGUCUCUGGUCCUUACUU-3′, SEQ ID NO: 40) and the sense strand ISIS 308746 (5′-AAGUAAGGACCAGAGACAAA-3′, SEQ ID NO: 39). The antisense strand, ISIS 342851, is comprised of a central RNA region with 4′-thioribose nucleosides at positions 1, 2, 3, 5, 16, 18, 19, and 20, indicated in bold. The sense strand, ISIS 308746, is comprised of ribonucleosides, and both strands of the ISIS 342851/308746 duplex have phosphodiester internucleoside linkages throughout.

PTEN mRNA levels in liver were measured at the end of the study using real-time PCR and RIBOGREEN™ RNA quantification reagent (Molecular Probes, Inc. Eugene, Oreg.) as taught in previous examples above. PTEN mRNA levels were determined relative to total RNA or GAPDH expression, prior to normalization to saline-treated control. Results are presented in the following table as the average % inhibition of mRNA expression for each treatment group, normalized to saline-injected control.

Target reduction by chemically modified
siRNAs targeted to PTEN in mouse liver

| | |
|---|---|
| Dose (mg/kg, | |
| administered | % Inhibition |

| Treatment | 2×/day) | Ribogreen | GAPDH |
|---|---|---|---|
| ISIS 116847 | 12.5 | 92 | 95 |
| | 6.25 | 92 | 95 |
| ISIS 342851/308746 | 25 | 11 | 18 |
| | 6.25 | 7 | 15 |
| ISIS 341391/341401 | 25 | 12 | 21 |
| | 6.25 | 2 | 9 |

As shown in the table, the oligonucleotides targeted to PTEN decreased mRNA levels relative to saline-treated controls. The mRNA levels measured for the ISIS 341391/341401 duplex are also suggestive of dose-dependent inhibition.

The effects of treatment with the RNA duplexes on plasma glucose levels were evaluated in the mice treated as described above. Glucose levels were measured using routine clinical analyzer instruments (eg. Ascencia Glucometer Elite XL, Bayer, Tarrytown, N.Y.). Approximate average plasma glucose is presented in the following table for each treatment group.

Effects of chemically modified siRNAs targeted
to PTEN on plasma glucose levels in normal mice

| | Dose (mg/kg, | |
|---|---|---|
| | administered | Plasma glucose |
| Treatment | 2×/day) | (mg/dL) |
| Saline | N/A | 186 |
| ISIS 116847 | 12.5 | 169 |
| | 6.25 | 166 |
| ISIS 342851/308746 | 25 | 167 |
| | 6.25 | 173 |
| ISIS 341391/341401 | 25 | 159 |
| | 6.25 | 182 |

To assess the physiological effects resulting from in vivo siRNA targeted to PTEN mRNA, the mice were evaluated at the end of the treatment period for plasma triglycerides, plasma cholesterol, and plasma transaminase levels. Routine clinical analyzer instruments (eg. Olympus Clinical Analyzer, Melville, N.Y.) were used to measure plasma triglycerides, cholesterol, and transaminase levels. Plasma cholesterol levels from animals treated with either dose of ISIS 116847 were increased about 20% over levels measured for saline-treated animals. Conversely, the cholesterol levels measured for animals treated with either the 25 mg/kg or the 6.25 mg/kg doses of the ISIS 341391/341401 duplex were decreased about 12% as compared to saline-treated controls. The other treatments did not cause substantial alterations in cholesterol levels. All of the treatment groups showed decreased plasma triglycerides as compared to saline-treated control, regardless of treatment dose.

Increases in the transaminases ALT and AST can indicate hepatotoxicity. The transaminase levels measured for mice treated with the siRNA duplexes were not elevated to a level indicative of hepatotoxicity with respect to saline treated control. Treatment with 12.5 mg/kg doses of ISIS 116847 caused approximately 7-fold and 3-fold increases in ALT and AST levels, respectively. Treatment with the lower doses (6.25 mg/kg) of ISIS 116847 caused approximately 4-fold and 2-fold increases in ALT and AST levels, respectively.

At the end of the study, liver, white adipose tissue (WAT), spleen, and kidney were harvested from animals treated with the oligomeric compounds and were weighed to assess gross organ alterations. Approximate average tissue weights for each treatment group are presented in the following table.

Effects of chemically modified siRNAs targeted
to PTEN on tissue weight in normal mice

| | Dose (mg/kg, | | | | |
|---|---|---|---|---|---|
| | administered | Liver | WAT | Spleen | Kidney |
| Treatment | 2×/day) | Tissue weight (g) | | | |
| Saline | N/A | 1.0 | 0.5 | 0.1 | 0.3 |
| ISIS 116847 | 12.5 | 1.1 | 0.4 | 0.1 | 0.3 |
| | 6.25 | 1.1 | 0.4 | 0.1 | 0.3 |
| ISIS 342851/308746 | 25 | 1.0 | 0.3 | 0.1 | 0.3 |
| | 6.25 | 0.9 | 0.4 | 0.1 | 0.3 |
| ISIS 341391/341401 | 25 | 1.0 | 0.3 | 0.1 | 0.3 |
| | 6.25 | 0.9 | 0.4 | 0.1 | 0.3 |

As shown, treatment with antisense oligonucleotides or siRNA duplexes targeted to PTEN did not substantially alter liver, WAT, spleen, or kidney weights in normal mice as compared to the organ weights of mice treated with saline alone.

### Example 39

### Stability of Alternating 2′-O-methyl/2′-fluoro siRNA Constructs in Mouse Plasma

Intact duplex RNA was analyzed from diluted mouse-plasma using an extraction and capillary electrophoresis method similar to those previously described (Leeds et al., Anal. Biochem., 1996, 235, 3643; Geary, Anal. Biochem., 1999, 274, 241-248. Heparin-treated mouse plasma, from 3-6 month old female Balb/c mice (Charles River Labs) was thawed from −80° C. and diluted to 25% (v/v) with phosphate buffered saline (140 mM NaCl, 3 mM KCl, 2 mM potassium phosphate, 10 mM sodium phosphate). Approximately 10 nmol of pre-annealed siRNA, at a concentration of 100 μM, was added to the 25% plasma and incubated at 37° C. for 0, 15, 30, 45, 60, 120, 180, 240, 360, and 420 minutes. Aliquots were removed at the indicated time, treated with EDTA to a final concentration of 2 mM, and placed on ice at 0° C. until analyzed by capillary gel electrophoresis (Beckmnan P/ACE MDQ-UV with eCap DNA Capillary tube). The area of the siRNA duplex peak was measured and used to calculate the percent of intact siRNA remaining. Adenosine triphosphate (ATP) was added at a concentration of 2.5 mM to each injection as an internal calibration standard. A zero time point was taken by diluting siRNA in phosphate buffered saline followed by capillary electrophoresis. Percent intact siRNA was plotted against time, allowing the calculation of a pseudo first-order half-life. Results are shown in the Table below. ISIS 338918 (UCUUAUCACCULUUAGCUCUA, SEQ ID NO: 54) and ISIS 338943 are umodified RNA strand with phosphodiester linkages throughout. ISIS 351831 is annotated as UₘC_{f}UₘU_{f}AₘU_{f}CₘA_{f}CₘC_{f}UₘU_{f}UₘA_{f}GₘC_{f}UₘC_{f}Uₘ and ISIS 351832 as A_{f}GₘA_{f}GₘC_{f}UₘA_{f}AₘA_{f}GₘF_{f}UₘG_{f}AₘU_{f}AₘA_{f}GₘA_{f} in other examples herein.

Stability of alternating 2′-O-methyl/2′-fluoro siRNA constructs in mouse plasma

| | | % Intact siRNA | | | | | | | | |
|---|---|---|---|---|---|---|---|---|---|---|
| | | Time (minutes) | | | | | | | | |
| Construct | SEQ ID NOs | 0 | 15 | 30 | 45 | 60 | 120 | 180 | 240 | 360 |
| 338918_338943 | 54 and 55 | 76.98 | 71.33 | 49.77 | 40.85 | 27.86 | 22.53 | 14.86 | 4.18 | 0 |
| 351831_351832 | 58 and 59 | 82.42 | 81.05 | 79.56 | 77.64 | 75.54 | 75.55 | 75.56 | 75.55 | 75 |

The parent (unmodified) construct is approximately 50% degraded after 30 minutes and nearly gone after 4 hours (completely gone at 6 hours). In contrast, the alternating 2′-O-methyl/2′-fluoro construct remains relatively unchanged and 75% remains even after 6 hours.

### Example 40

### In Vivo Inhibition of Survivin Expression in a Human Glioblastoma Xenograft Tumor Model

The U-87MG human glioblastoma xenograft tumor model (Kiaris et al., 2000, May-June; 2(3):242-50) was used to demonstrate the antitumor activity of selected compositions of the present invention. A total of 8 CD1 nu/nu (Charles River) mice were used for each group. For implantation, tumor cells were trypsinized, washed in PBS and resuspended in PBS at 4×10⁶ cells/mL in DMEM. Just before implantation, animals were irradiated (450 TBI) and the cells were mixed in Matrigel (1:1). A total of 4×10⁶ tumor cells in a 0.2 mL volume were injected subcutaneously (s.c.) in the left rear flank of each mouse. Treatment with the selected double stranded compositions (dissolved in 0.9% NaCl, injection grade), or vehicle (0.9% NaCl) was started 4 days post tumor cell implantation. The compositions were administered intravenously (i.v.) in a 0.2 mL volume eight hours apart on day one and four hours apart on day two. Tissues (tumor, liver, kidney, serum) were collected two hours after the last dose. Tumors from eight animals from each group were homogenized for western evaluation. Survivin levels were determined and compared to saline controls.

SEQ ID No/

| | |
|---|---|
| ISIS No | Sequence 5′-3′ |

| | |
|---|---|
| 24/343868 (as) | UUUGAAAAUGUUGAUCUCC |
| 25/343867 (s) | GGAGAUCAACAUUUUCAAA |
| 24/355713 (as) | UₘU_{f}UₘG_{f}AₘA_{f}AₘA_{f}UₘG_{f}UₘU_{f}GₘA_{f}UₘC_{f}UₘC_{f} |
| | Cₘ |
| 25/355714 (s) | G_{f}GₘA_{f}GₘA_{f}UₘC_{f}AₘA_{f}CₘA_{f}UₘU_{f}UₘU_{f}CₘA_{f}Aₘ |
| | A_{f} |
| 24/353537 (as) | UₜUₜUₜGAAAAUGUUGAUCUₜCₜCₜ |
| 25/343868 (s) | GGAGAUCAACAUUUUCAAA |
| 24/352506 (as) | UUUGAAₘAₘAUGₘUₘUGAUCUₘCₘCₘ |
| 25/352514 (s) | GGₑAGₑAUₑCAₑACₑAUₑUUₑUCₑAAₑA |

| | |
|---|---|
| Double stranded construct | Activity |

| Antisense | Sense | % Inhibition of Survivin |
|---|---|---|
| 343868 | 343867 | none |
| 355713 | 355714 | 60 |
| 353537 | 343868 | 48 |
| 352506 | 352514 | 44 |

The data demonstrate that modified chemistries can be used to stabilize the constructs resulting in activity not seen with the unmodified construct.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed:

1. A composition comprising first and second chemically synthesized oligomeric compounds wherein:
at least a portion of the first oligomeric compound is complementary to and capable of hybridizing to a selected nucleic acid target;
a portion of from about 12 to about 24 nucleosides of the first oligomeric compound is complementary to the second oligomeric compound;
one of the first and second oligomeric compounds is an asymmetric gapped oligomeric compound;
the other of the first and second oligomeric compounds is a fully modified oligomeric compound; and
the composition optionally further comprises one or more overhangs, phosphate moieties, conjugate groups or capping groups.

2. The composition of claim 1 wherein the asymmetric gapped oligomeric compound comprises a contiguous sequence of nucleosides divided into an internal region flanked by two external regions wherein:
the sugar groups within each region are identical and the sugar groups of each region are different from the sugar groups of each other region;
the nucleosides of the internal region are 1-D-ribonucleosides or sugar modified nucleosides and the nucleosides of the external regions are sugar modified nucleosides; and
the sugar modified nucleosides are each independently selected from 2′-modified nucleosides, 4′-thio modified nucleosides, 4′-thio-2′-modified nucleosides and nucleosides having bicyclic sugar moieties.

3. The composition of claim 2 wherein the internal region of the asymmetric gapped oligomeric compound is a sequence of β-D-ribonucleosides.

4. The composition of claim 2 wherein the internal region of the asymmetric gapped oligomeric compound is a sequence of sugar modified nucleosides.

5. The composition of claim 4 wherein the sugar modified nucleosides are 2′-F modified nucleosides or 4′-thio modified nucleosides.

6. The composition of claim 2 wherein at least one of the external regions of the asymmetric gapped oligomeric compound is a sequence of 2′-modified nucleosides.

7. The composition of claim 6 wherein each of the external regions of the asymmetric gapped oligomeric compound is a sequence of 2′-modified nucleosides.

8. The composition of claim 6 wherein the 2′-modifications of each external region having a sequence of 2′-modified nucleosides are, independently, selected from halogen, allyl, amino, azido, —O-allyl, —O—C₁-C₁₀ alkyl, —OCF₃, —O—(CH₂)₂—OCH₃, —O(CH₂)₂—SCH₃, —O—(CH₂)₂—ON(Rₘ)(Rₙ) or —O—CH₂—C(═O)N(Rₘ)(Rₙ), where each Rₘ and Rₙ is, independently, H, an amino protecting group or substituted or unsubstituted —C₁-C₁₀ alkyl.

9. The composition of claim 8 wherein the 2′-modifications of each external region having a sequence of 2′-modified nucleosides is independently selected from —F, —OCH₃ or —O—(CH₂)₂—OCH₃.

10. The composition of claim 2 wherein one of the external regions of the asymmetric gapped oligomeric compound is a sequence of 4′-thio modified nucleosides.

11. The composition of claim 2 wherein one of the external regions of the asymmetric gapped oligomeric compound is a sequence of 4′-thio-2′-modified nucleosides.

12. The composition of claim 11 wherein the 2′-modifications of the 4′-thio-2′-modified nucleosides are selected from halogen, allyl, amino, azido, —O-allyl, —O—C₁-C₁₀ alkyl, —OCF₃, —O-(CH₂)₂—OCH₃, —O(CH₂)₂—SCH₃, —O—(CH₂)₂—ON(Rₘ)(Rₙ) and —O—CH₂—C(═O)N(Rₘ)(Rₙ), where each Rₘ and Rₙ is, independently, H, an amino protecting group or substituted or unsubstituted —C₁-C₁₀ alkyl.

13. The composition of claim 12 wherein each of the 2′-modifications is —F, —OCH₃, —OCF₃ or —O—(CH₂)₂—OCH₃.

14. The composition of claim 13 wherein each of the 2′-modifications is —OCH₃ or —O—(CH₂)₂—OCH₃.

15. The composition of claim 2 wherein one of the external regions of the asymmetric gapped oligomeric compound is a sequence of bicyclic sugar moieties.

16. The composition of claim 15 wherein each of the bicyclic sugar moieties has a 2′-O—(CH₂)ₙ-4′ bridge wherein n is 1 or 2.

17. The composition of claim 1 wherein the first oligomeric compound is the asymmetric gapped oligomeric compound.

18. The composition of claim 17 wherein one of the external regions of the asymmetric gapped oligomeric compound is a sequence of 4′-thio modified nucleosides and the other external region is a sequence of 2′-modified nucleosides.

19. The composition of claim 18 wherein the external region located at the 5′-end of the asymmetric gapped oligomeric compound is a sequence of 2′-OCH₃, 2′-F or 4′-thio modified nucleosides.

20. The composition of claim 18 wherein the 2′-modified nucleosides are 2′-OCH₃ or 2′-F modified nucleosides.

21. The composition of claim 20 wherein the 2′-modified nucleosides are 2′-OCH₃ modified nucleosides.

22. The composition of claim 1 wherein the second oligomeric compound is the asymmetric gapped oligomeric compound.

23. The composition of claim 22 wherein at least one of the external regions of the asymmetric gapped oligomeric compound is a sequence of 2′-modified nucleosides wherein the 2′-modification is selected from halogen, allyl, amino, azido, —O-allyl, —O—C₁-C₁₀ alkyl, —OCF₃, —O—(CH₂)₂—OCH₃, —O(CH₂)₂—SCH₃, O—(CH₂)₂—ON(Rₘ)(Rₙ) and O—CH₂—C(═O)N(Rₙ)(Rₙ), where each Rₘ and Rₙ is, independently, H, an amino protecting group or substituted or unsubstituted —C₁-C₁₀ alkyl.

24. The composition of claim 23 wherein the 2′-modification is selected from allyl, —O-allyl, —O—C₂-C₁₀ alkyl, —O—(CH₂)₂—OCH₃ or —O(CH₂)₂—SCH₃.

25. The composition of claim 24 wherein the 2′-modification is —O—(CH₂)₂—OCH₃.

26. The composition of claim 2 wherein each of the external regions of the asymmetric gapped oligomeric compound independently comprise from 1 to about 6 nucleosides.

27. The composition of claim 2 wherein each of the external regions of the asymmetric gapped oligomeric compound independently comprise from 1 to about 4 nucleosides.

28. The composition of claim 2 wherein each of the external regions of the asymmetric gapped oligomeric compound independently comprise from 1 to about 3 nucleosides.

29. The composition of claim 1 wherein each nucleoside of the fully modified oligomeric compound is a sugar modified nucleoside and wherein each sugar modification is the same.

30. The composition of claim 29 wherein each sugar modified nucleoside of the fully modified oligomeric compound is selected from 2′-modified nucleosides, 4′-thio modified nucleosides, 4′-thio-2′-modified nucleosides and nucleosides having bicyclic sugar moieties.

31. The composition of claim 30 wherein each sugar modified nucleoside is a 2′-modified nucleoside.

32. The composition of claim 31 wherein each sugar modified nucleoside is a 2′-OCH₃ or a 2′-F modified nucleoside.

33. The composition of claim 32 wherein each sugar modified nucleoside is a 2′-OCH₃ modified nucleoside.

34. The composition of claim 32 wherein each sugar modified nucleoside is a 2′-F modified nucleoside.

35. The composition of claim 32 wherein one or both of the 3′ and 5′-termini of the fully modified oligomeric compound is a β-D-ribonucleoside.

36. The composition of claim 1 having at least 2 phosphorothioate internucleoside linking groups at the 3′-end of the first oligomeric compound.

37. The composition of claim 36 having about 7 phosphorothioate internucleoside linking groups at the 3′-end of the first oligomeric compound.

38. The composition of claim 1 wherein the first oligomeric compound further comprises a 5′-thiophosphate group.

39. The composition of claim 1 wherein each of the internucleoside linking groups of the first and the second oligomeric compounds are independently selected from phosphodiester or phosphorothioate.

40. The composition of claim 1 wherein each of the first and second oligomeric compounds independently comprises from about 12 to about 30 nucleosides.

41. The composition of claim 1 wherein each of the first and second oligomeric compounds independently comprises from about 17 to about 23 nucleosides.

42. The composition of claim 1 wherein each of the first and second oligomeric compounds independently comprises from about 19 to about 21 nucleosides.

43. The composition of claim 1 wherein the first and the second oligomeric compounds form a complementary antisense/sense siRNA duplex.

44. The composition of claim 1 wherein the first oligomeric compound is an antisense oligomeric compound and the second oligomeric compound is a sense oligomeric compound.

45. A method of inhibiting gene expression comprising contacting one or more cells, a tissue or an animal with a composition of claim 1.
